(19) United States
(12) Patent Application Publication
(10) Pub. No.: US 2026/0092083 A1
(43) Pub. Date: Apr. 2, 2026

(54) VANCOMYCIN DERIVATIVES WITH SIDEROPHORE MODIFICATION AS NOVEL REPURPOSED ANTIBIOTICS TO COMBAT GRAM-NEGATIVE BACTERIAL INFECTION

(71) Applicant: RESEARCH TRIANGLE INSTITUTE, RESEARCH TRIANGLE PARK, NC (US)

(72) Inventors: Chunyang JIN, Apex, NC (US); Bruce Edward BLOUGH, Raleigh, NC (US); Dongliang GUAN, Durham, NC (US)

(73) Assignee: RESEARCH TRIANGLE INSTITUTE, RESEARCH TRIANGLE PARK, NC (US)

(21) Appl. No.: 19/162,234

(22) PCT Filed: Mar. 6, 2024
(86) PCT No.: PCT/US2024/018655

§ 371 Date: Sep. 4, 2025

Related U.S. Application Data
(60) Provisional application No. 63/451,337, filed on Mar. 10, 2023.

(51) Int. Cl. C07K 9/00; A61K 38/00; A61P 31/04

(52) U.S. Cl. CPC C07K 9/008; A61P 31/04; A61K 38/00

(57) ABSTRACT

The present disclosure provides novel vancomycin derivatives that act as antimicrobials against Gram-negative bacteria. The compounds of the present disclosure are believed to be useful for the treatment of diseases caused by antimicrobial resistant bacteria, including Acinetobacter baumannii and Escherichia coli.

### CROSS-REFERENCE TO RELATED APPLICATIONS

This application claims the benefit of U.S. Provisional Application No. 63/451,337, filed Mar. 10, 2023, which is incorporated by reference herein in its entirety.

### FEDERALLY SPONSORED RESEARCH

This invention was made with government support under 75N93021C00002 awarded by the National Institute of Health. The government has certain rights in the invention.

### FIELD

The present disclosure relates to novel vancomycin-siderophore conjugates and pharmaceutical compositions thereof, and methods of treatment of diseases caused by antimicrobial resistant pathogens. The pharmaceutical compositions described herein are useful as a broad-spectrum antibiotic to fight Gram-negative bacterial infections.

### BACKGROUND OF THE DISCLOSURE

Antimicrobial resistance (AMR) has become a prominent problem. Threatening public health and global development, AMR has been declared one of the top 10 global public health threats by the World Health Organization (WHO), causing severe morbidity and mortality. Sec Antimicrobial Resistance: A Top Ten Global Public Health Threat. EClinicalMedicine 2021, 41, 101221. According to a CDC report in 2019, it was estimated that more than 2.6 million antibiotic-resistant infections and nearly 44,000 deaths occurred each year in the United States alone. Sec, Antibiotic Resistance Threats in The United States. AR Threats Report 2019. Across the globe, AMR kills an estimated 700,000 people each year, and some experts predict that if no efforts are made to halt drug resistance or develop new antibiotics, this number could rise to 10 million by 2050. Sec Willyard, Drug-Resistant Bacteria Ranked. Nature 2017, 543 (7643). In 2017, the WHO published a threat list of global priority pathogens comprising 12 species of bacteria with critical, high, and medium antibiotic resistance. See, e.g., Asokan, et al., WHO Global Priority Pathogens List: A Bibliometric Analysis of Medline-PubMed for Knowledge Mobilization to Infection Prevention and Control Practices in Bahrain. Oman Medical Journal 2019, 34 (3), 184-193, and Kwon, et al., The Post-Antibiotic Era Is Here. Science 2021, 373 (6554), 471. Among them, three kinds of Gram-negative bacterium, namely Acinetobacter baumannii (carbapenem), Pseudomonas aeruginosa (carbapenem) and Enterobacteriaceae, extended-spectrum β-lactamase-producing (carbapenem) are ranked in top 3. In Antibiotic Resistance Threats in the United States 2019 Threat Report, the Gram-negative bacteria Carbapenem-resistant Acinetobacter, Carbapenem-resistant Enterobacteriaceae and Drug-resistant Neisseria gonorrhoeae are ranked 1st, 4th and 5th place, respectively, in the Urgent Threats list. Despite there being 76 antibacterial clinical candidates at present, there are relatively few clinically differentiated antibacterial agents in late-stage clinical development, especially against critical-priority pathogens. Sec, e.g., Butler, et. al., Analysis of the Clinical Pipeline of Treatments for Drug-Resistant Bacterial Infections: Despite Progress, More Action Is Needed. Antimicrobial Agents and Chemotherapy 2022, 66 (3), c0199121. Therefore, new antibiotics development especially towards critical-priority Gram-negative pathogens are needed to combat antibiotic resistance.

The Gram-negative bacterial envelope structure has three principal layers: the outer membrane, the periplasmic space, and the inner membrane. The outer membrane barrier hinders many antibiotics from effectively permeating to arrive at the targets, complicating new antibiotic development towards Gram-negative bacteria.

Vancomycin, as the first and most representative member in the glycopeptide antibiotic family, was regarded as “last resort” for the treatment of severe resistant Gram-positive bacterial infection, such as Staphylococcus aureus, Enterococcus spp. and Clostridium difficile. See e.g., Binda, et. al., Old and New Glycopeptide Antibiotics: Action and Resistance. Antibiotics (Basel) 2014, 3 (4), 572-594. Vancomycin binds to the D-Ala-D-Ala moiety of the bacterial cell wall precursor Lipid II to inhibit the subsequent transglycosylation and transpeptidation, leading to bacterial lysis and death. See e.g., Nicolaou, et. al., Chemistry, Biology, and Medicine of the Glycopeptide Antibiotics. Angewandte Chemie Internation Edition 1999, 38 (15), 2096-2152. After 30 years of extensive use, clinical resistance to vancomycin has emerged and presents an urgent need for the development of next generation glycopeptide antibiotics. See, e.g., Uttley, et al., Vancomycin-Resistant Enterococci. Lancet 1988, 1 (8575-6), 57-58, and Leclercq, et al., Plasmid-Mediated Resistance to Vancomycin and Teicoplanin in Enterococcus Faecium. New England Journal of Medicine 1988, 319 (3), 157-161.

In recent years, it has become an attractive option to repurpose “old” drugs to treat both common and rare diseases. See e.g., Pushpakom, et. al., Drug Repurposing: Progress, Challenges and Recommendations. Nature Reviews Drug Discovery 2019, 18 (1), 41-58. Vancomycin is a cell wall biosynthesis inhibitor effective in Gram-positive bacteria only, due to its inability to penetrate the outer membrane of Gram-negative bacteria. The peptidoglycan layer, vancomycin's target, is considered the “Achilles Heel” for bacteria as it plays a very important role for bacterial survival. In Gram-negative bacteria, it is located in the periplasmic space with much thinner layers in comparison to Gram-positive bacterial peptidoglycan layers. See e.g., Sibinelli-Sousa, et. al., Targeting the Achilles 'heel of Bacteria: Different Mechanisms to Break Down the Peptidoglycan Cell Wall during Bacterial Warfare. Journal of Bacteriology 2021, 203 (7), e00478-20. Facilitating vancomycin penetration of the outer membrane to the periplasmic space appears critical and significant to overcome resistance of Gram-negative bacteria.

The “Trojan horse” strategy has previously been widely and successfully applied to antibiotic design in which iron-chelating siderophores are conjugated to an antibiotic or antimicrobial agent to enhance uptake and antibacterial potency. Sec e.g., Kong, et. al., An Overview of Recent Progress in Siderophore-Antibiotic Conjugates. European Journal of Medicinal Chemistry 2019, 182, 111615, Jun, et. al., Research Progress of Antibiotics Conjugated with Siderophores. Chinese Journal of Organic Chemistry 2020, 40 (10), 3026-3043, and Negash, et. al., Siderophore-Antibiotic Conjugate Design: New Drugs for Bad Bugs? Molecules 2019, 24 (18), 3314, each of which is incorporated herein by reference with regard to such background teaching. Iron is an essential nutrient for bacterial growth, replication, and metabolism. To obtain enough iron to sustain their growth and perform various physiological activities during infection, bacteria have evolved a series of systems that can take up iron through the secretion of small organic compounds to act as iron carriers extracellularly. These iron carriers, siderophores, obtain various forms of extracellular iron and move them into the cell by the active transport system. Sec e.g., Page, The Role of Iron and Siderophores in Infection, and the Development of Siderophore Antibiotics. Clinical Infectious Diseases 2019, 69 (Suppl 7), S529-S537. The most representative example of a siderophore-antibiotic conjugate is Cefiderocol, which was newly approved by the FDA in 2019.

As shown in the present application, the “Trojan horse” strategy to link vancomycin to a synthetic siderophore provides a mechanism for vancomycin to penetrate the outer membrane via active uptake by the iron transport system. These vacomycin derivatives are able to bypass the Gram-negative cell wall, converting vancomycin into a broad-spectrum antibiotic to fight Gram-negative bacteria. Underlying the importance of rationally designing the siderophore type and size to overcome the Gram-negative outer membrane permeability barrier.

Thus, there remains a need for novel antibiotics to address antimicrobial resistance.

### BRIEF SUMMARY OF THE DISCLOSURE

Thus, the present disclosure is directed to compounds and pharmaceutically acceptable salts thereof, pharmaceutical compositions, and methods useful in treating bacterial infection. The compounds and salts thereof, compositions, and methods described herein are useful in treating or preventing bacterial infections or diseases, including infections caused by Gram-negative and Gram-positive bacteria.

In one embodiment described herein, the compounds of the present disclosure are represented by formula (I), or a pharmaceutical acceptable salt thereof:

In one embodiment the compounds of the present disclosure selected from the group consisting of:

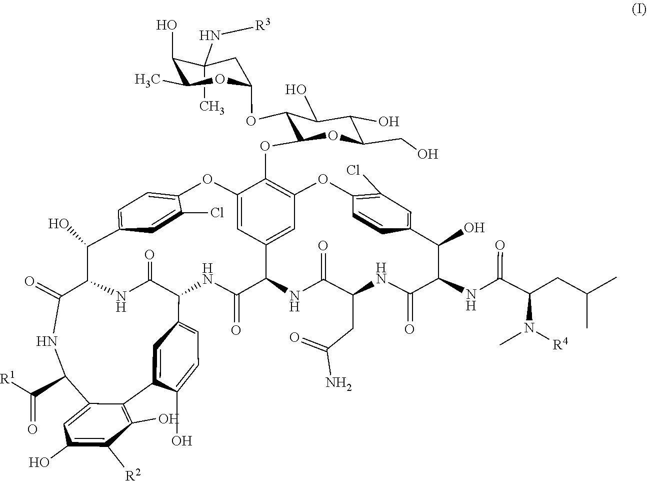

wherein:
(i) R¹ is

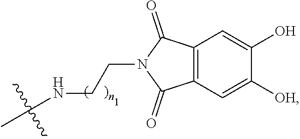

and each of R², R³, and R⁴, is H;
(ii) R¹ is

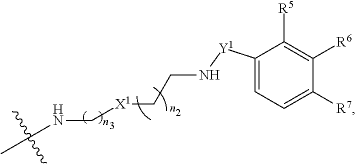

and each of R², R³, and R⁴, is H;
(iii) R¹ is

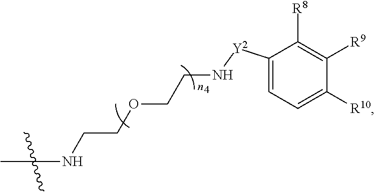

and each of R², R³, and R⁴, is H;
(iv) R¹ is

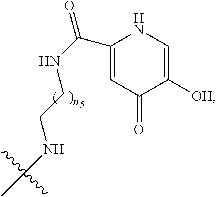

and each of R², R³, and R⁴, is H;
(v) R¹ is OH; R² is

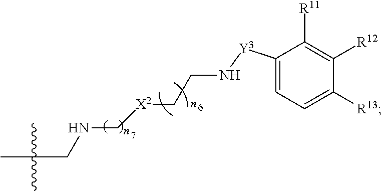

and each of R³ and R⁴ are H;
(vi) R¹ is OH; R³ is

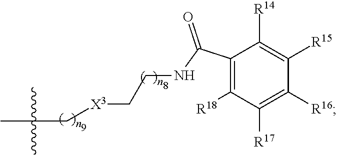

and each of R² and R⁴ are H; or
(vii) R¹ is OH; R⁴ is

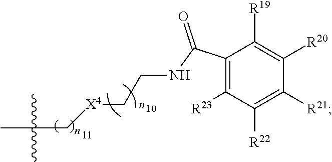

and each of R² and R³ are H; and wherein:
X¹ is a bond or

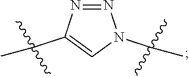 [SMILES: *c1cn(*)nn1]

X² is a bond or

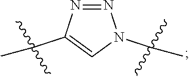 [SMILES: *c1cn(*)nn1]

X³ is a bond or

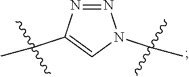 [SMILES: *c1cn(*)nn1]

X⁴ is a bond or

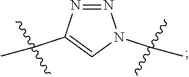 [SMILES: *c1cn(*)nn1]

Y¹ is C(O) or SO₂;
Y² is C(O) or SO₂;
Y³ is C(O) or SO₂;
n₁ is 1-5;
n₂ is 1-10;
n₃ is 0-5;
n₄ is 1-5;
n₅ is 1-5;
n₆ is 1-10;
n₇ is 0-5;
n₈ is 0-5;
n₉ is 0-5;
n₁₀ is 0-5;
n₁₁ is 0-5;
R⁵ is selected from the group consisting of hydrogen, halogen, and OH;
R⁶ is selected from the group consisting of hydrogen, halogen, and OH;
R⁷ is selected from the group consisting of hydrogen, halogen, and OH;
R⁸ is selected from the group consisting of hydrogen, halogen, and OH;
R⁹ is selected from the group consisting of hydrogen, halogen, and OH;
R¹⁰ is selected from the group consisting of hydrogen, halogen, and OH;
R¹¹ is selected from the group consisting of hydrogen, halogen, and OH;
R¹² is selected from the group consisting of hydrogen, halogen, and OH;
R¹³ is selected from the group consisting of hydrogen, halogen, and OH;
R¹⁴ is selected from the group consisting of hydrogen, halogen, and OH;
R¹⁵ is selected from the group consisting of hydrogen, halogen, and OH;
R¹⁶ is selected from the group consisting of hydrogen, halogen, and OH;
R¹⁷ is selected from the group consisting of hydrogen, halogen, and OH;
R¹⁸ is selected from the group consisting of hydrogen, halogen, and OH;
R¹⁹ is selected from the group consisting of hydrogen, halogen, and OH;
R²⁰ is selected from the group consisting of hydrogen, halogen, and OH;
and R²¹ is selected from the group consisting of hydrogen, halogen, and OH.

In another aspect the compound described herein, the halogen is Cl.

In another aspect the compound described herein, R1 is

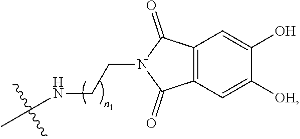

and each of R2, R3, and R4, is H.

In another aspect the compound described herein n1 is 1.

In another aspect the compound described herein n1 is 3.

In another aspect the compound described herein R1 is

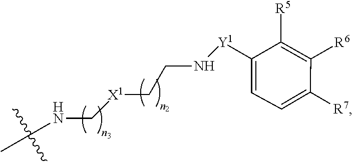

and each of R2, R3, and R4, is H.

In another aspect the compound described herein n2 is 1, n3 is 0, X1 is a bond, Y1 is C(O), R5 is Cl, R6 is OH, and R7 is OH.

In another aspect the compound described herein n2 is 2 n3 is 0, X1 is a bond, Y1 is C(O), R5 is Cl, R6 is OH, and R7 is OH.

In another aspect the compound described herein n2 is 3, n3 is 0, X1 is a bond, Y1 is

C(O), R5 is Cl, R6 is OH, and R7 is OH.

In another aspect the compound described herein n2 is 4, n3 is 0, X1 is a bond, Y1 is C(O), R5 is Cl, R6 is OH, and R7 is OH.

In another aspect the compound described herein n2 is 5, n3 is 0, X1 is a bond, Y1 is C(O), R5 is Cl, R6 is OH, and R7 is OH.

In another aspect the compound described herein n2 is 6, n3 is 0, X1 is a bond, Y1 is C(O), R5 is Cl, R6 is OH, and R7 is OH.

In another aspect the compound described herein n2 is 7, n3 is 0, X1 is a bond, Y1 is C(O), R5 is Cl, R6 is OH, and R7 is OH.

In another aspect the compound described herein n2 is 2, n3 is 0, X1 is a bond, Y1 is C(O), R5 is OH, R6 is OH, and R7 is H.

In another aspect the compound described herein n2 is 3, n3 is 0, X1 is a bond, Y1 is C(O), R5 is OH, R6 is OH, and R7=H.

In another aspect the compound described herein n2 is 1, n3 is 1, X1 is

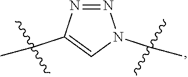 [SMILES: *c1cn(*)nn1]

### Y1 is C(O), R5 is OH, R6 is OH, and R7 is H.

In another aspect the compound described herein n2 is 1, n3 is 2, X1 is

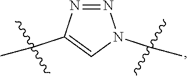 [SMILES: *c1cn(*)nn1]

### Y1 is C(O), R5 is OH, R6 is OH, and R7 is H.

In another aspect the compound described herein n2 is 2, n3 is 0, X1 is a bond, Y1 is SO2, R5 is H, R6 is OH, and R7 is OH.

In another aspect the compound described herein n2 is 3, n3 is 0, X1 is a bond, Y1 is SO2, R5 is H, R6 is OH, and R7 is OH.

In another aspect the compound described herein n2 is 2, n3 is 1, X1 is

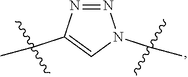 [SMILES: *c1cn(*)nn1]

### Y1 is C(O), R5 is Cl, R6 is OH, and R7 is OH.

In another aspect the compound described herein R1 is

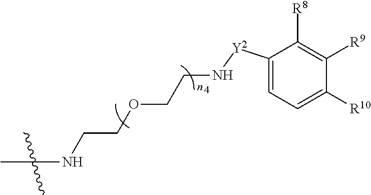

and each of R², R³, and R⁴, is H.

In another aspect the compound described herein n4 is 1, Y2 is C(O), R8 is Cl, R9 is OH, and R10 is OH.

In another aspect the compound described herein n4 is 2, Y2 is C(O), R8 is Cl, R9 is OH, and R10 is OH.

In another aspect the compound described herein n4 is 2, Y2 is SO2, R8 is H, R9 is OH, and R10 is OH.

In another aspect the compound described herein R1 is

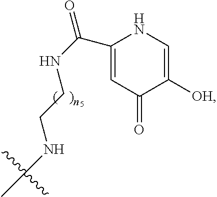

and each of R², R³, and R⁴, is H.

In another aspect the compound described herein n₅ is 2.

In another aspect the compound described herein n₅ is 3.

In another aspect the compound described herein R1 is OH; R2 is

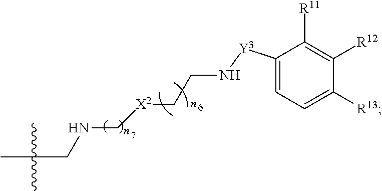

and each of R3 and R4 are H.

In another aspect the compound described herein n6 is 2, n7 is 0, X2 is a bond, Y3 is C(O), R11 is Cl, R12 is OH, and R13 is OH.

In another aspect the compound described herein n6 is 3, n7 is 0, X2 is a bond, Y3 is C(O), R11 is Cl, R12 is OH, and R13 is OH.

In another aspect the compound described herein n6 is 1, n7 is 0, X2 is a bond, Y3 is C(O), R11 is OH, R12 is OH, and R13 is H.

In another aspect the compound described herein n6 is 1, n7 is 0, X2 is a bond, Y3 is SO2, R11 is H, R12 is OH, and R13 is OH.

In another aspect the compound described herein n₆ is 2, n₇ is 1, X² is

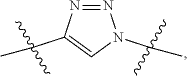 [SMILES: *c1cn(*)nn1]

Y³ is C(O), R¹¹ is Cl, R¹² is OH, and R¹³ is H.

In another aspect the compound described herein R1 is OH; R3 is

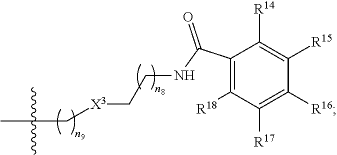

and each of R2 and R4 are H.

In another aspect the compound described herein n8 is 1, n9 is 0, X3 is a bond, R14 is Cl, R15 is OH, R16 is OH, R17 is H, and R18 is H.

In another aspect the compound described herein n8 is 2, n9 is 0, X3 is a bond, R14 is Cl, R15 is OH, R16 is OH, R17 is H, and R18 is H.

In another aspect the compound described herein n8 is 2, n9 is 3, X3=is

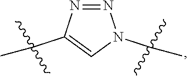 [SMILES: *c1cn(*)nn1]

### R14 is H, R15 is H, R16 is OH, R17 is OH, and R18 is Cl.

In another aspect the compound described herein OH; R4 is

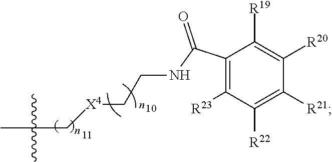

and each of R2 and R3 are H.

In another aspect the compound described herein n10 is 1, n11 is 0, X4 is a bond, R19 is Cl, R20 is OH, R21 is OH, R22 is H, and R23 is H.

In another aspect the compound described herein n10 is 1, n11 is 3, X4 is

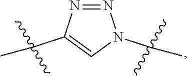 [SMILES: *c1cn(*)nn1]

### R19 is H, R20 is H, R21 is OH, R22 is OH, and R23 is Cl.

In another embodiment described herein is a compound or a pharmaceutically acceptable salt thereof selected from the group consisting of:

| Com- | |
|---|---|
| pound # | Compound |
| 1 | 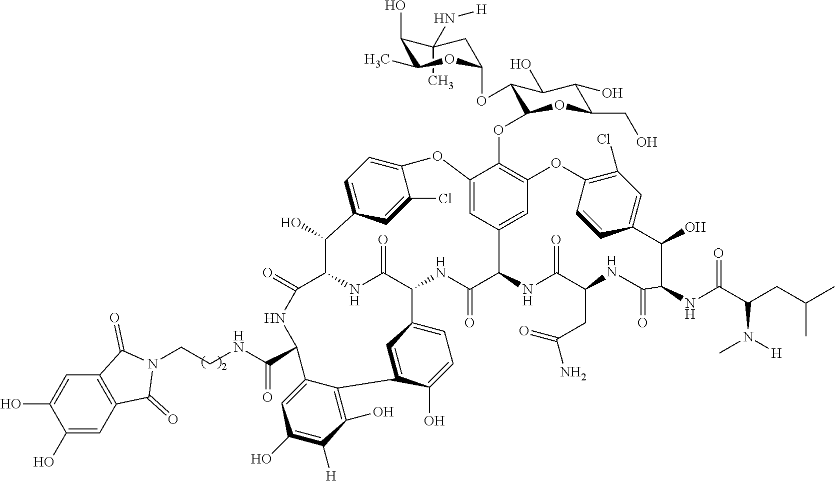 |
| 2 | 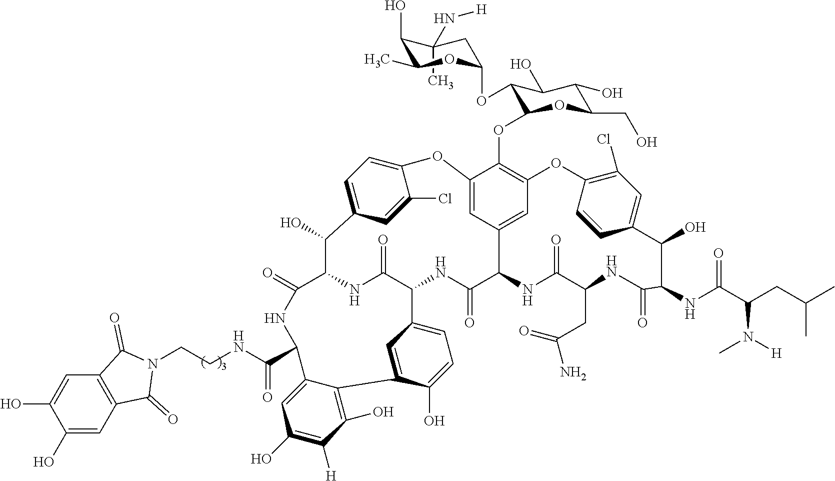 |
| 3 | 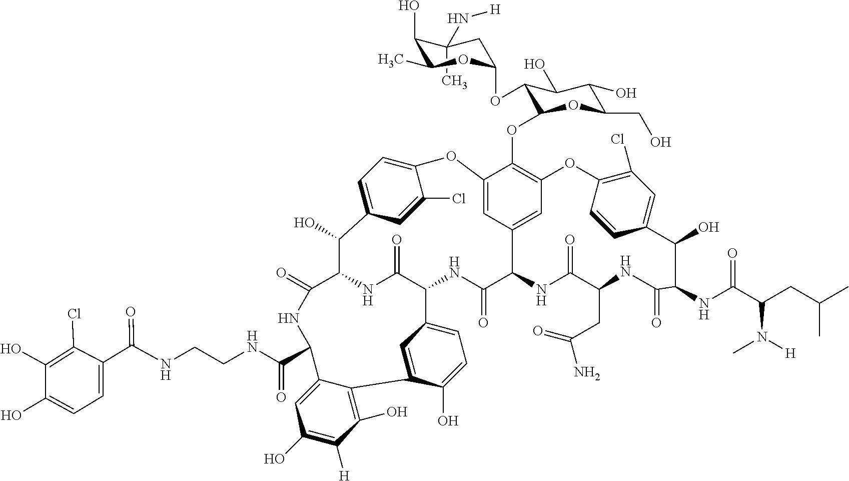 |
| 4 | 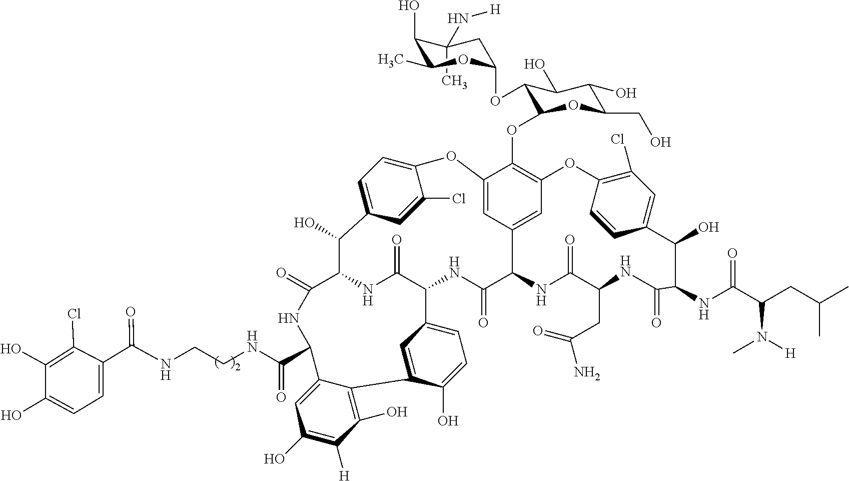 |
| 5 | 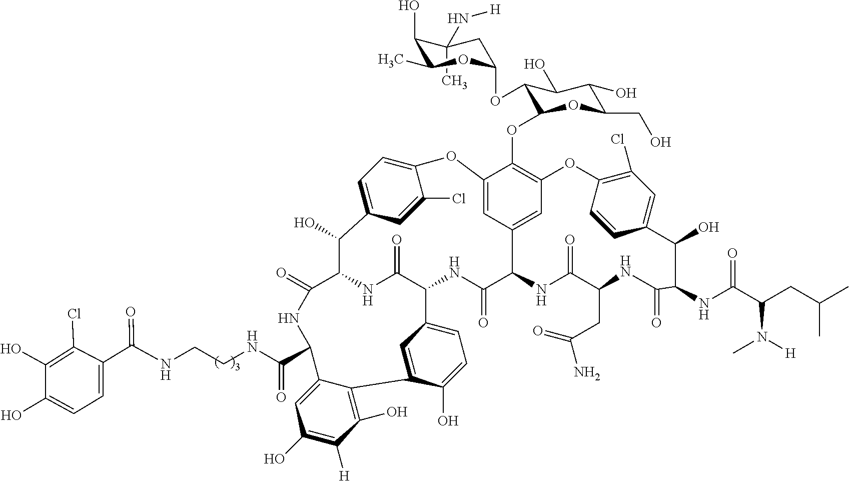 |
| 6 | 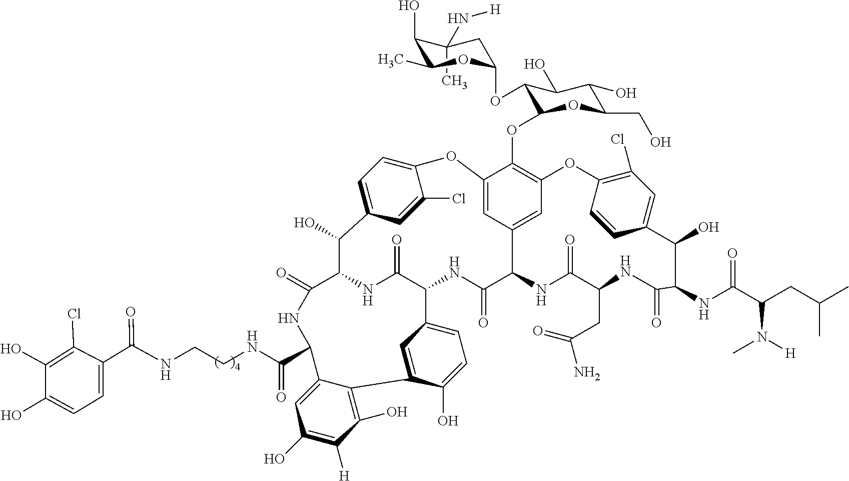 |
| 7 | 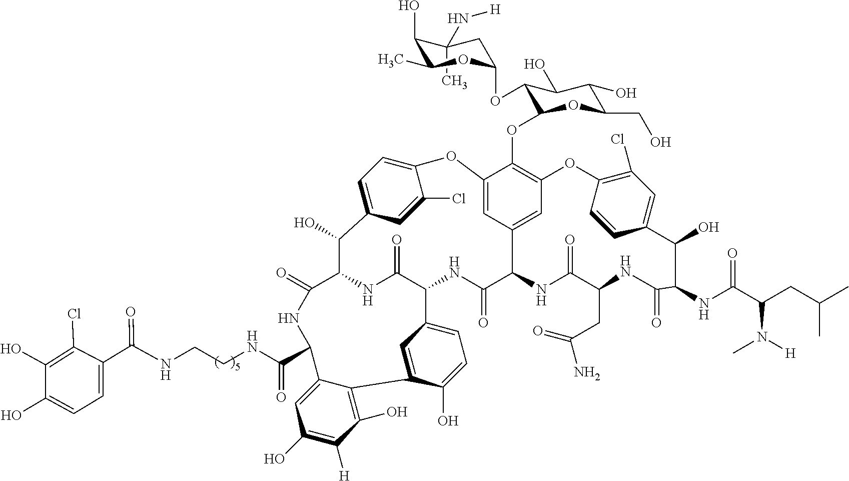 |
| 8 | 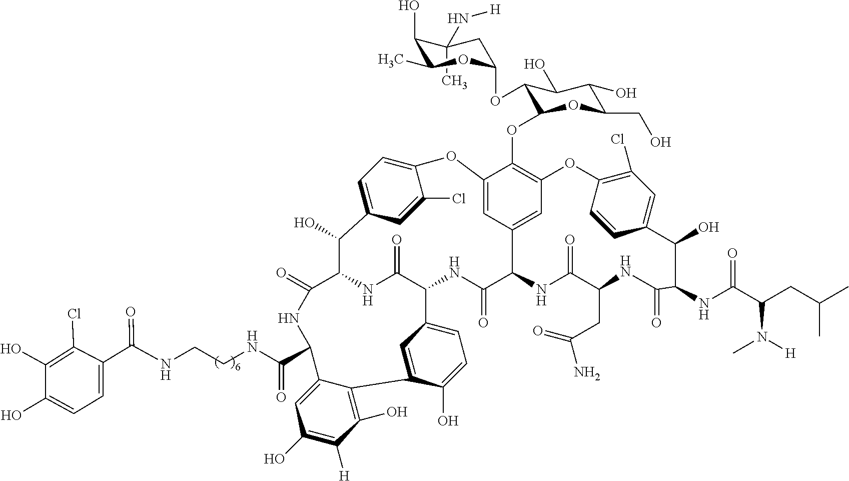 |
| 9 | 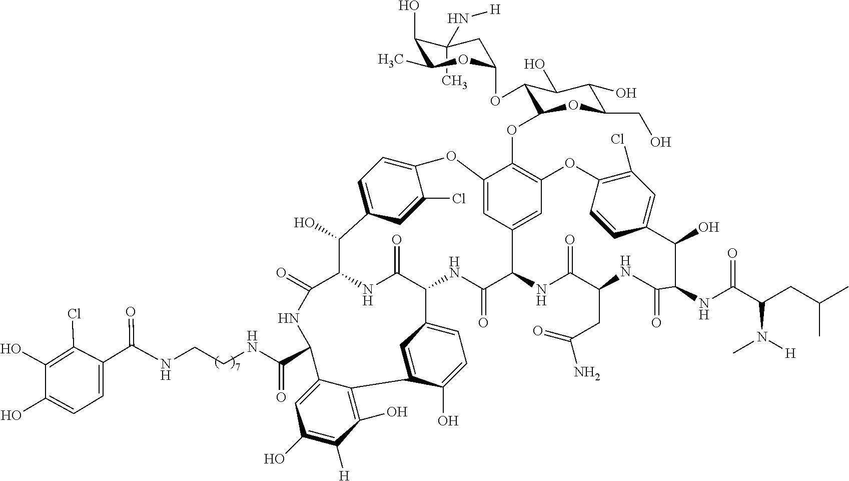 |
| 10 | 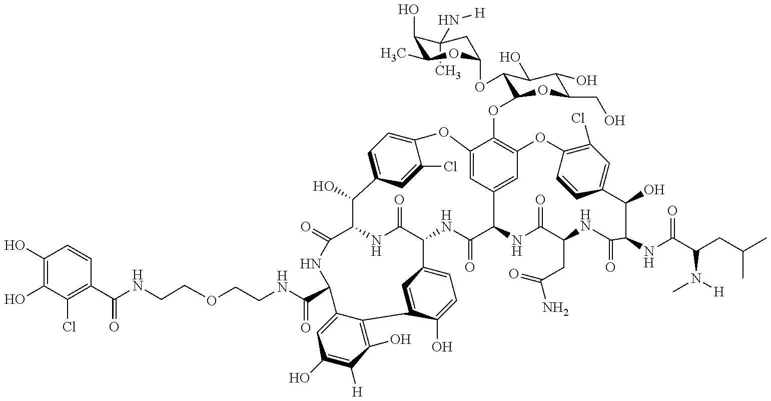 |
| 11 | 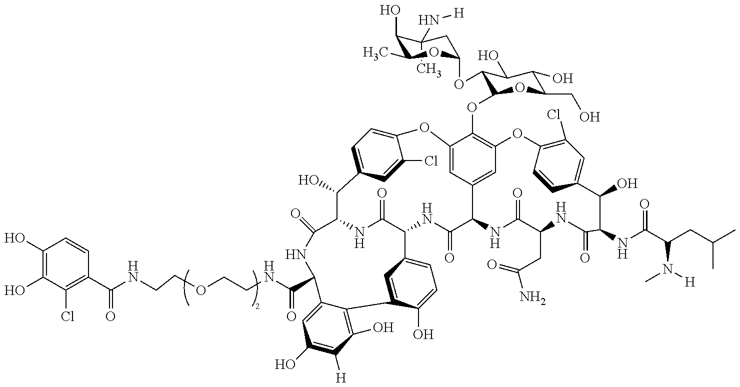 |
| 12 | 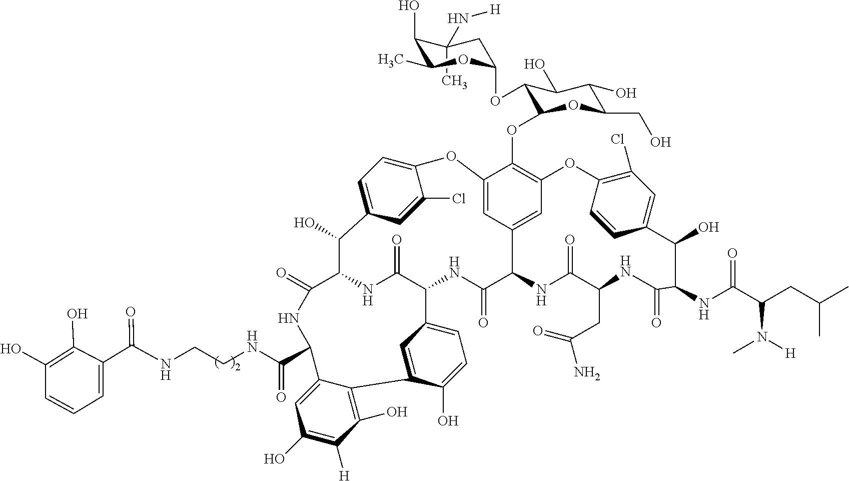 |
| 13 | 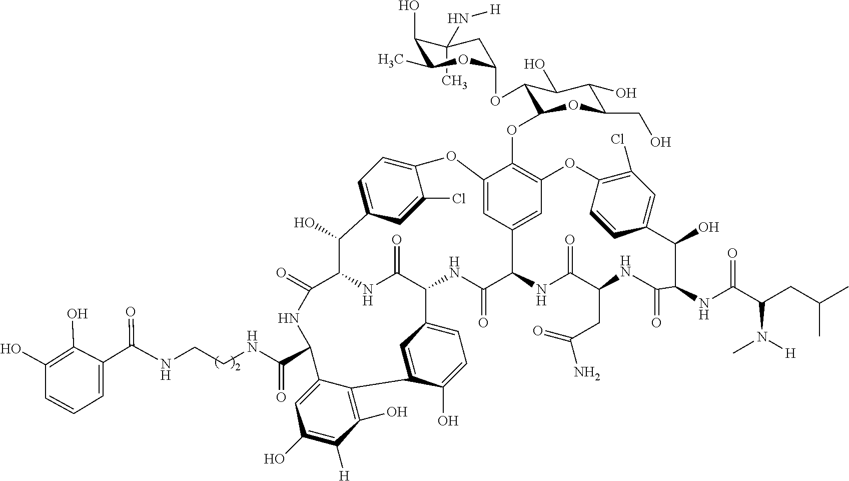 |
| 14 | 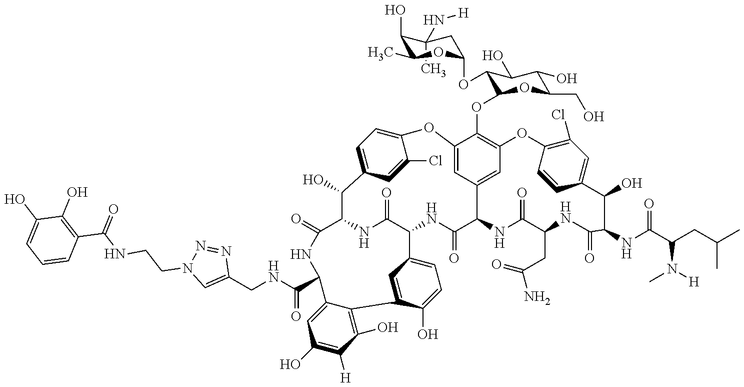 |
| 15 | 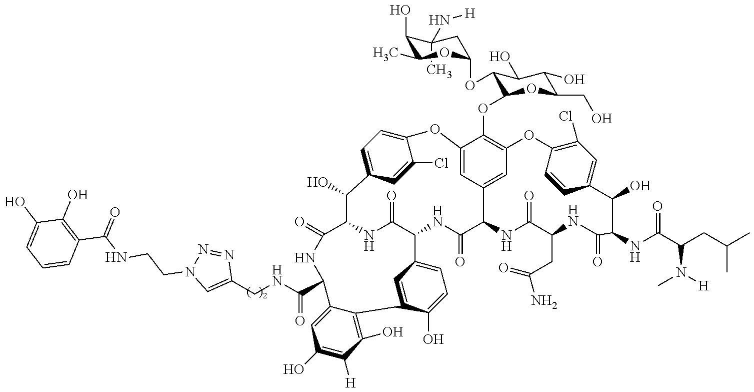 |
| 16 | 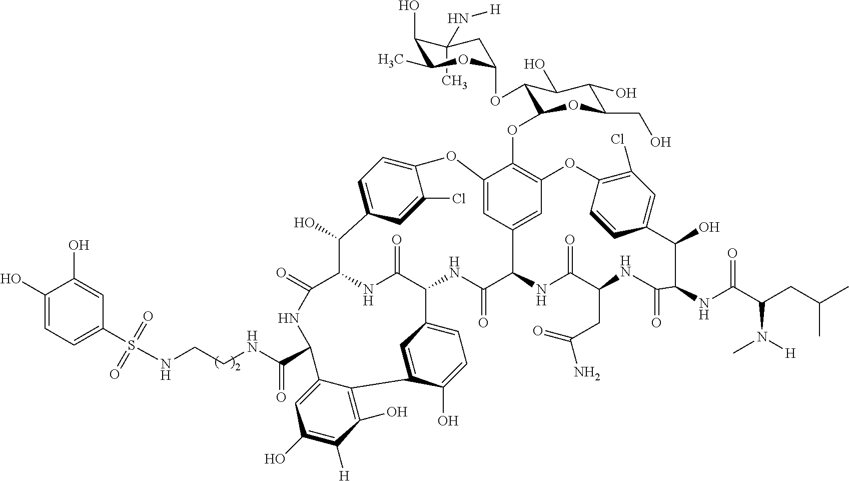 |
| 17 | 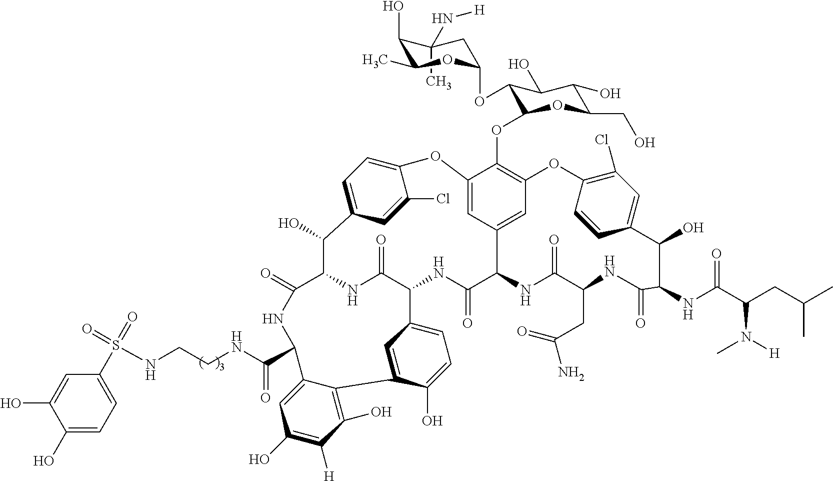 |
| 18 | 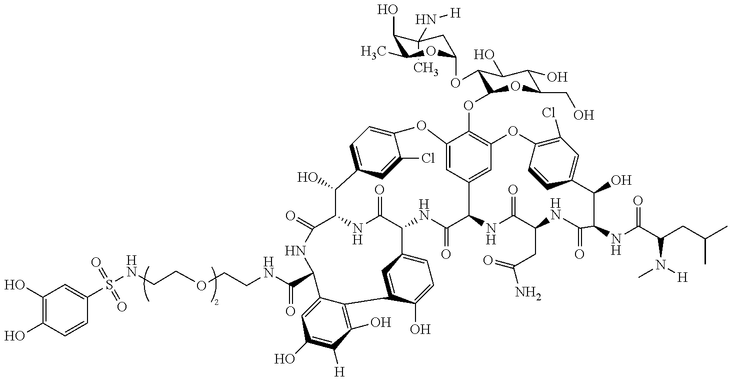 |
| 19 | 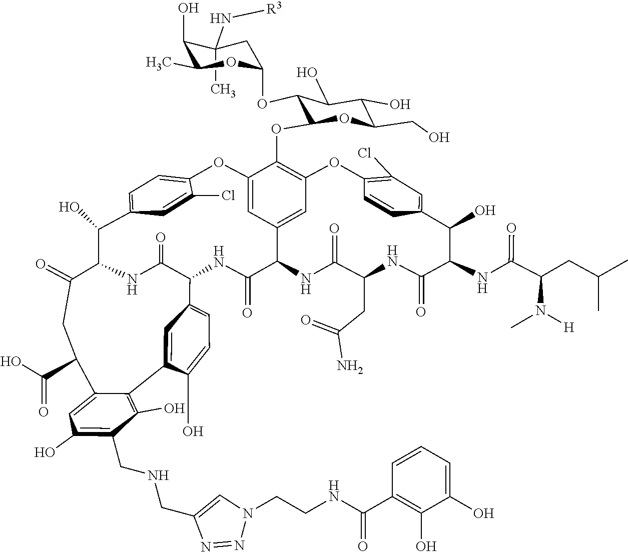 |
| 20 | 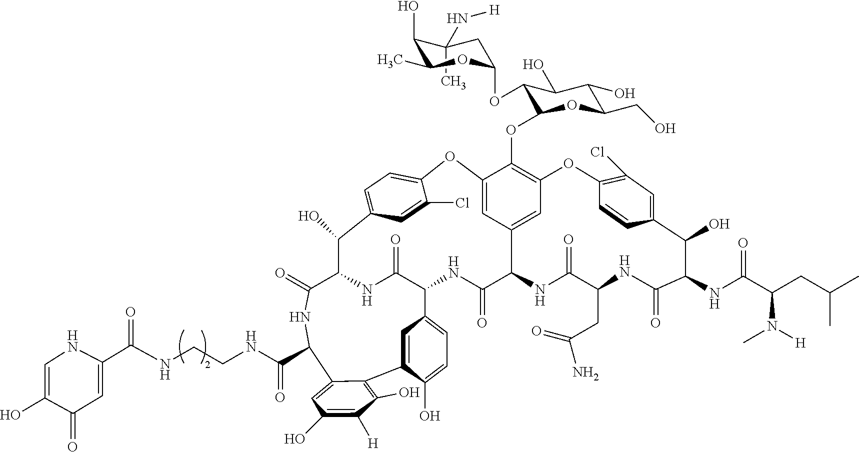 |
| 21 | 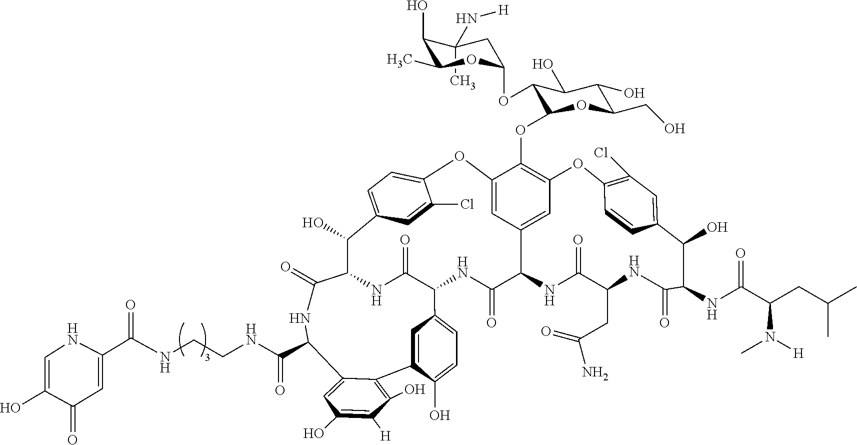 |
| 22 | 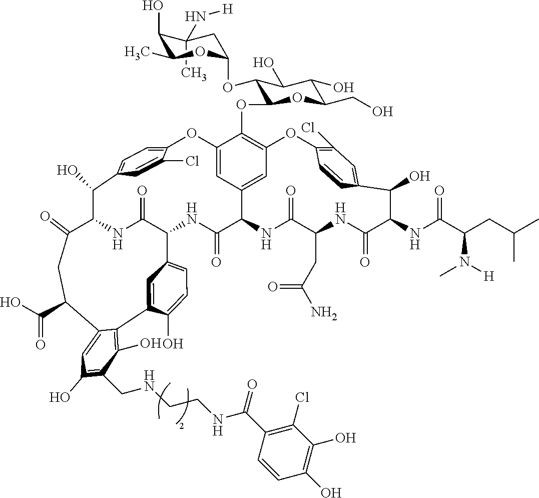 |
| 23 | 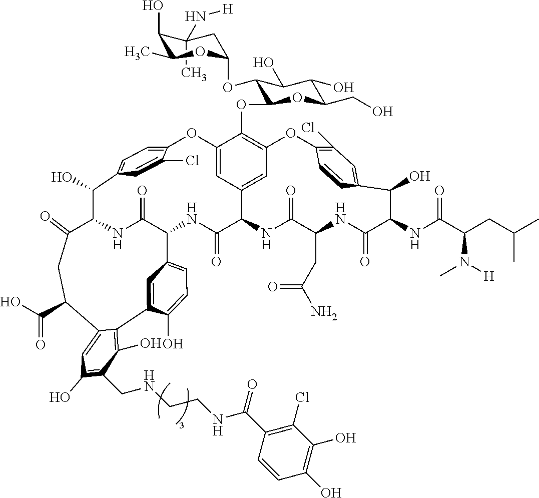 |
| 24 | 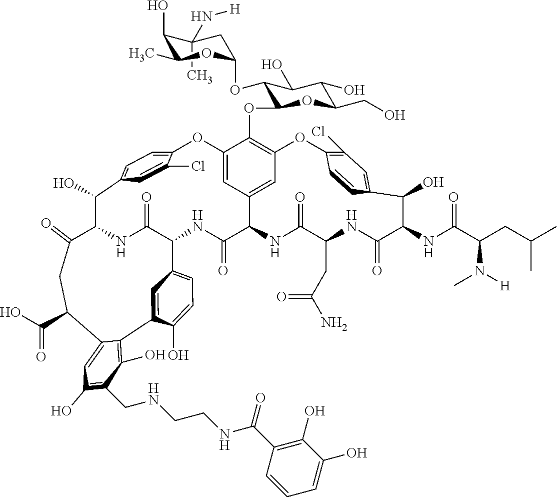 |
| 25 | 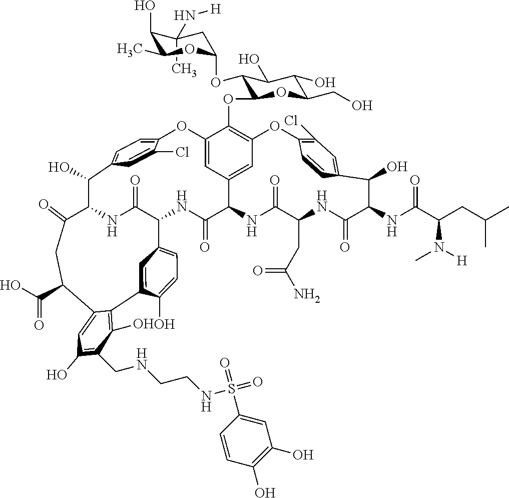 |
| 26 | 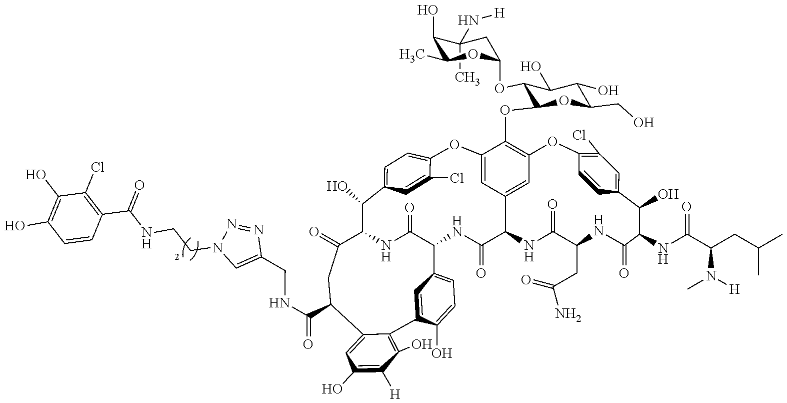 |
| 27 | 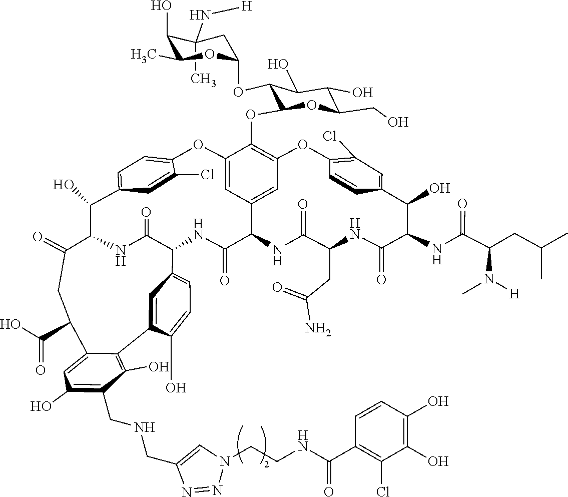 |
| 28 | 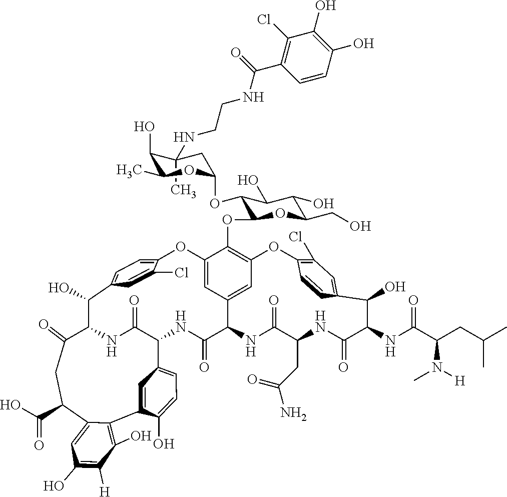 |
| 29 | 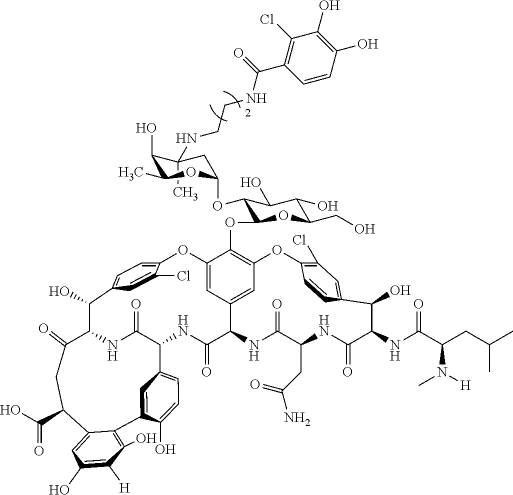 |
| 30 | 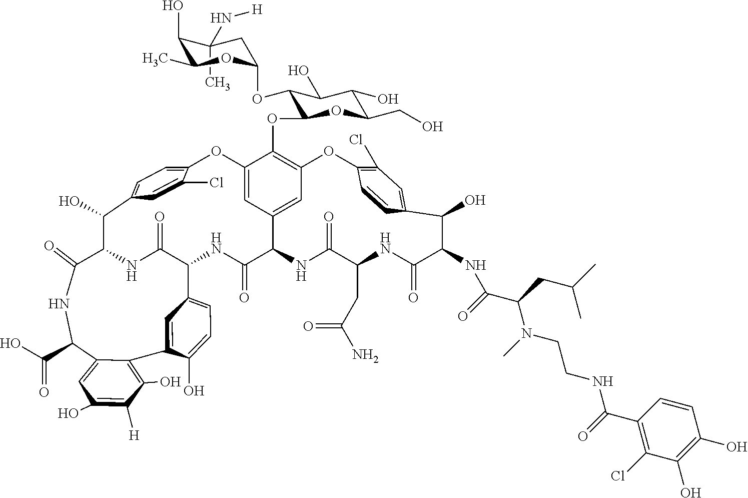 |
| 31 | 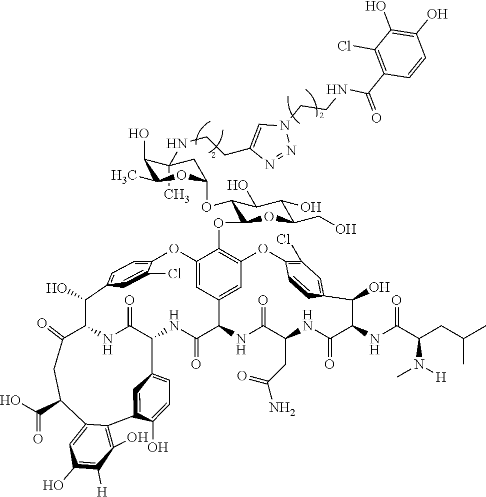 |
| | and |
| 32 | 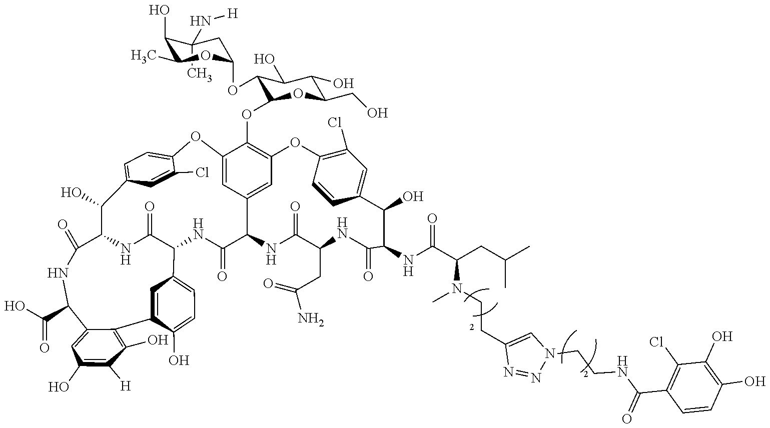 |

or a pharmaceutically acceptable salt thereof.

In other aspects, the present disclosure also relates to a pharmaceutical composition including any of the compounds of the disclosure or a pharmaceutically acceptable salt thereof. Another aspect of the present disclosure relates to a pharmaceutical composition including any of the compounds of the disclosure and a pharmaceutically acceptable carrier.

A further aspect of the present disclosure relates to a pharmaceutical composition including a therapeutically effective amount of the compounds of the disclosure or a pharmaceutically acceptable salt thereof and one or more pharmaceutically acceptable carriers or vehicles.

According to another aspect, the compounds of the disclosure or the pharmaceutical composition of the disclosure may be used in the treatment of a bacterial infectious disease. Another aspect includes a method of treating a bacterial infection in a patient in need thereof, including administering to the patient the compounds of the disclosure or the compositions of the disclosure. A further aspect relates to a method of treating a bacterial infection in a patient in need thereof, including administering to the patient the compounds of the disclosure or the compositions of the disclosure in combination with another treatment agent.

In still other aspects, a method of treating a bacterial infection in a patient in need thereof, by administering to the patient the compounds of the disclosure or the compositions of the disclosure in combination with another treatment agent further includes when the bacteria are a Gram-positive bacteria or Gram-negative bacteria. In a further aspect, a method of treating a bacterial infection in a patient in need thereof, by administering to the patient the compounds of the disclosure or the compositions of the disclosure in combination with another treatment agent further includes when the bacteria are a Gram-positive bacteria. In another aspect, a method of treating a bacterial infection in a patient in need thereof, by administering to the patient the compounds the disclosure or the compositions of the disclosure in combination with another treatment agent further includes when the bacteria are a Gram-negative bacteria. Additional aspects include a method of treating a bacterial infection in a patient in need thereof, by administering to the patient the compounds of the disclosure or the compositions of the disclosure in combination with another treatment agent further when the bacteria are Acinetobacter baumannii Gram-negative bacteria and Escherichia coli Gram-negative bacteria.

Another aspect of the present disclosure relates to a method of controlling, treating, or reducing the advancement, severity, or effects of a bacterial infection in a patient, including administering to said patient a therapeutically effective around of the compounds the disclosure or the pharmaceutical compostions of the disclosure.

In other aspects, the present disclosure relates to a method of decreasing or inhibiting bacterial quantity in a biological sample, including contacting said biological sample with a compound of the disclosure or a composition of the disclosure.

In still other aspects, the present disclosure relates to the use of a compound of the disclosure or the pharmaceutical compositions the disclosure, in the manufacture of a medicament for the treatment of a bacterial infectious disease in a patient in needed thereof. In further aspects, the use of a compound of the disclosure or the pharmaceutical compositions of the disclosure, in the manufacture of a medicament for the treatment of a bacterial infectious disease in a patient in needed thereof includes the treatment of a bacterial infectious disease associated with Gram-positive bacteria or Gram-negative bacteria. An additional aspect relates to the use of a compound of the disclosure or the pharmaceutical compositions of the disclosure, in the manufacture of a medicament for the treatment of a bacterial infectious disease in a patient in needed thereof when the treatment of a bacterial infectious disease is associated with Gram-positive bacteria. Another aspect relates to the use of a compound of the disclosure or the pharmaceutical compositions of the disclosure, in the manufacture of a medicament for the treatment of a bacterial infectious disease in a patient in needed thereof when the treatment of a bacterial infectious disease is associated with Gram-negative bacteria. Additionally, the use of a compound of the disclosure or the pharmaceutical compositions of the disclosure, in the manufacture of a medicament for the treatment of a bacterial infectious disease in a patient in needed thereof includes the treatment of a bacterial infectious disease associated with Gram-negative bacteria including Acinetobacter baumannii and Escherichia coli.

### DETAILED DESCRIPTION OF THE DISCLOSURE

### General

The present disclosure is directed to compounds and salts thereof, compositions and methods useful in the treatment of bacterial infections. Treatment of bacterial infections, as used herein in its various forms, is intended to encompass killing a bacterium or inhibiting, reducing or preventing the growth thereof, comprising contacting said bacterium with a compound as described herein. The ability of a compound to treat bacterial infections, may be demonstrated in an assay (e.g., minimum inhibitory concentration).

In one embodiment described herein, the compounds of the present disclosure are represented by formula (I), or a pharmaceutical acceptable salt thereof:

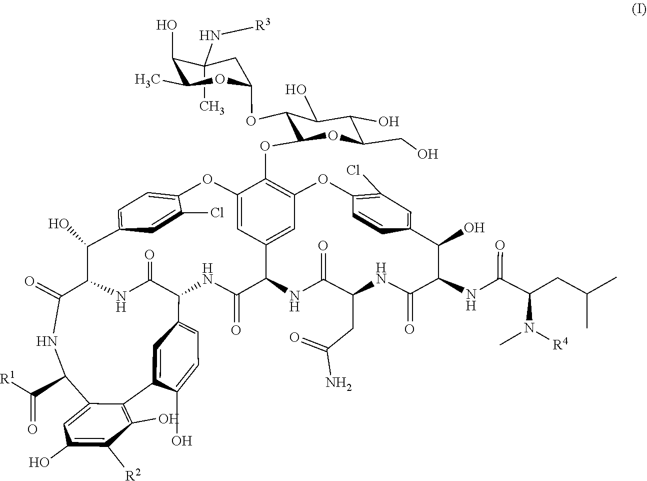

where R¹ is selected from the group consisting of

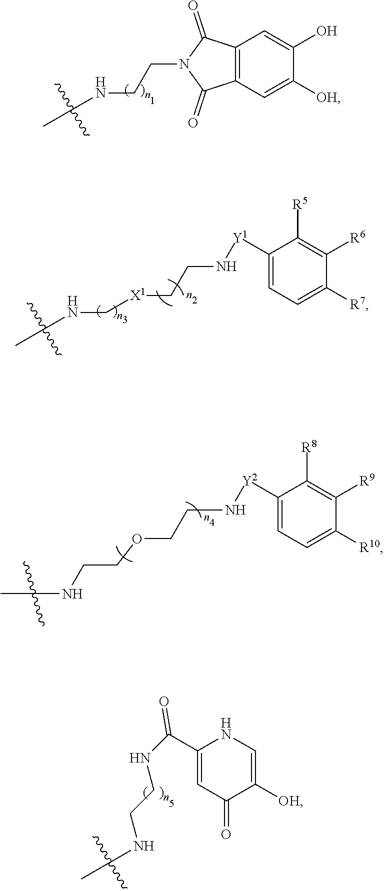

and OH; and each of R², R³, and R⁴, is H.

If R¹ is OH, R² may be

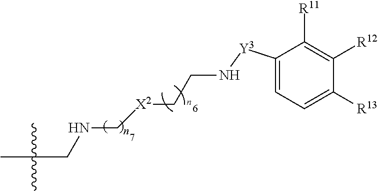

with each of R³ and R⁴ being H. Alternatively, if R¹ is OH, R³ may be

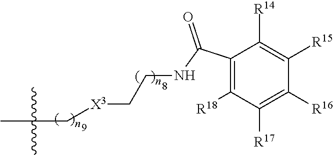

with each of R² and R⁴ being H. In addition to the above, if R¹ is OH, R⁴ may be

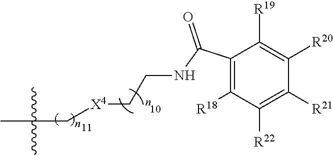

with each of R² and R³ being H.
X¹ is selected from the group consisting of a bond or

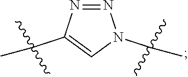 [SMILES: *c1cn(*)nn1]

X² is selected from the group consisting of a bond or

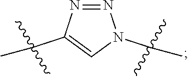 [SMILES: *c1cn(*)nn1]

X³ is selected from the group consisting of a bond or

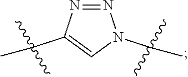 [SMILES: *c1cn(*)nn1]

X⁴ is selected from the group consisting of a bond or

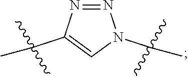 [SMILES: *c1cn(*)nn1]

Y¹ is selected from the group consisting of C(O) or SO₂;
Y² is selected from the group consisting of C(O) or SO₂;
Y³ is selected from the group consisting of C(O) or SO₂;
n₁ is an integer from 1 to 5 inclusive;
n₂ is an integer from 1 to 10 inclusive;
n₃ is an integer from 0 to 5 inclusive;
n₄ is an integer from 1 to 5 inclusive;
n₅ is an integer from 1 to 5 inclusive;
n₆ is an integer from 1 to 10 inclusive;
n₇ is an integer from 0 to 5 inclusive;
n₈ is an integer from 0 to 5 inclusive;
n₉ is an integer from 0 to 5 inclusive;
n₁₀ is an integer from 0 to 5 inclusive;
n₁₁ is an integer from 0 to 5 inclusive;
R⁵ is selected from the group consisting of hydrogen, halogen, and OH;
R⁶ is selected from the group consisting of hydrogen, halogen, and OH;
R⁷ is selected from the group consisting of hydrogen, halogen, and OH;
R⁸ is selected from the group consisting of hydrogen, halogen, and OH;
R⁹ is selected from the group consisting of hydrogen, halogen, and OH;
R¹⁰ is selected from the group consisting of hydrogen, halogen, and OH;
R¹¹ is selected from the group consisting of hydrogen, halogen, and OH;
R¹² is selected from the group consisting of hydrogen, halogen, and OH;
R¹³ is selected from the group consisting of hydrogen, halogen, and OH;
R¹⁴ is selected from the group consisting of hydrogen, halogen, and OH;
R¹⁵ is selected from the group consisting of hydrogen, halogen, and OH;
R¹⁶ is selected from the group consisting of hydrogen, halogen, and OH;
R¹⁷ is selected from the group consisting of hydrogen, halogen, and OH;
R¹⁸ is selected from the group consisting of hydrogen, halogen, and OH;
R¹⁹ is selected from the group consisting of hydrogen, halogen, and OH;
R²⁰ is selected from the group consisting of hydrogen, halogen, and OH;
and R²¹ is selected from the group consisting of hydrogen, halogen, and OH.

In one aspect of the compound as described herein, the halogen is Cl.

In one aspect of the compound as described herein R¹ is

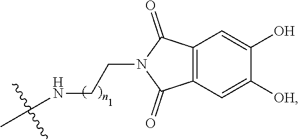

and each of R², R³, and R⁴, is H. In another aspect n₁ is 1. In a further aspect n₁ is 3.

In another aspect of the compound as described herein R¹ is

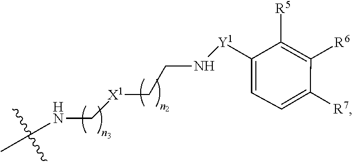

and each of R², R³, and R⁴, is H. In another aspect n₃ is 0, X¹ is a bond, Y¹ is C(O), R⁵ is Cl, R⁶ is OH, and R⁷ is OH. In a further aspect n₂ is 1, 2, 3, 4, 5, 6, or 7.

In one aspect of the compound as described herein R¹ is

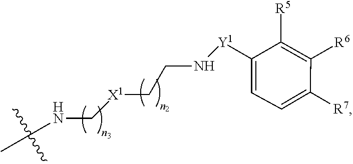

and each of R², R³, and R⁴, is H. In another aspect n₃ is 0, X¹ is a bond, Y¹ is C(O), R⁵ is OH, R⁶ is OH, and R⁷ is H. In a further aspect n₂ is 2 or 3.

In one aspect of the compound as described herein R¹ is

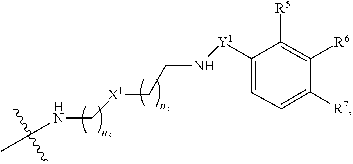

and each of R², R³, and R⁴, is H. Another aspect n₂ is 1, X¹ is

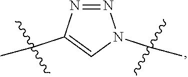 [SMILES: *c1cn(*)nn1]

Y¹ is C(O), R⁵ is OH, R⁶ is OH, and R⁷ is H. In a further aspect n₃ is 1 or 2.

In another aspect of the compound as described herein R¹ is

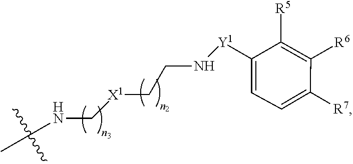

and each of R², R³, and R⁴, is H. In another aspect n₃ is 0, X¹ is a bond, Y¹ is SO₂, R⁵ is H, R⁶ is OH, and R⁷ is OH. In a further aspect n₂ is 2 or 3.

In one aspect of the compound as described herein R¹ is

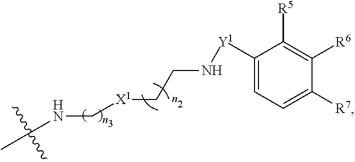

and each of R², R³, and R⁴, is H. In another aspect n₂=2, n₃=1, X¹ be

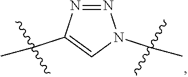 [SMILES: *c1cn(*)nn1]

Y¹ is C(O), R⁵ is Cl, R⁶ is OH, and R⁷ is OH.

In one aspect of the compound as described herein R¹ is

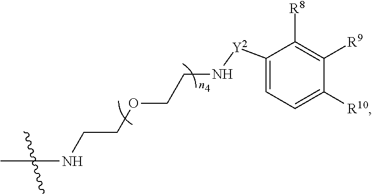

and each of R², R³, and R⁴, is H. In another aspect Y² is C(O), R⁸ is Cl, R⁹ is OH, and R¹⁰ is OH. In a further aspect n₄=1 or 2.

In another aspect of the compound as described herein R¹ is

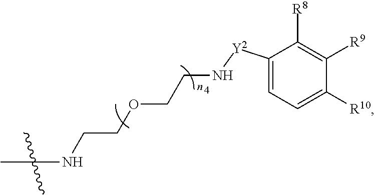

and each of R², R³, and R⁴, is H. In another aspect n₄=2 Y² is SO₂, R⁸ is H, R⁹ is OH, and R¹⁰ is OH.

In one aspect of the compound as described herein R¹ is

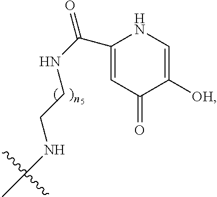

and each of R², R³, and R⁴, is H. In a further aspect n₅=2 or 3.

In one aspect of the compound as described herein R¹ is OH, R² is

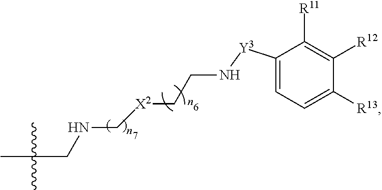

and each of R³ and R⁴ is H. In another aspect n₇=0, X² is a bond, Y³ is C(O), R¹¹ is Cl, R¹² is OH, and R¹³ is OH. In a further aspect n₆=2 or 3.

In one aspect of the compound as described herein R¹ is OH, R² is

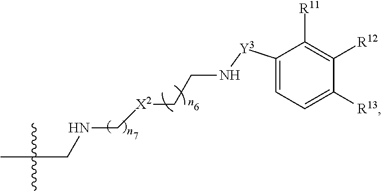

and each of R³ and R⁴ is H. In another aspect n₆=1, n₇=0, X² is a bond, Y³ is C(O), R¹¹ is OH, R¹² is OH, and R¹³ is H.

In one aspect of the compound as described herein R¹ is OH, R² is

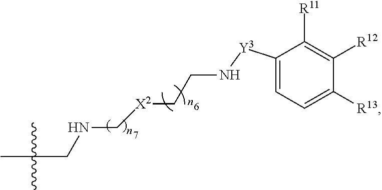

and each of R³ and R⁴ is H. In another aspect n₆=1, n₇=0, X² is a bond, Y³ is SO₂, R¹¹ is H, R¹² be OH, and R¹³ be H.

In another aspect of the compound as described herein R¹ is OH, R² is

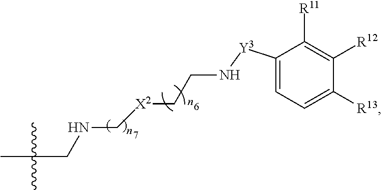

and each of R³ and R⁴ is H. In another aspect n₆=2, n₇=1, X² is

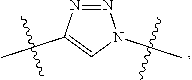 [SMILES: *c1cn(*)nn1]

Y³ is C(O), R¹¹ is Cl, R¹² is OH, and R¹³ is H.

In one aspect of the compound as described herein R¹ is OH, R³ is

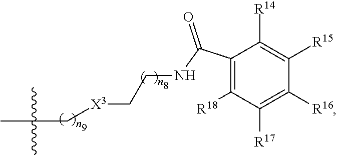

and each of R² and R⁴ is H. In another aspect n₉=0, X³ is a bond, R¹⁴ is Cl, R¹⁵ is OH, R¹⁶ is OH, R¹⁷ is H, and R¹⁸ is H. In a further aspect n₈=1 or 2.

In one aspect of the compound as described herein R¹ is OH, R³ is

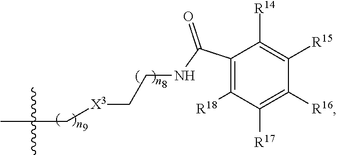

and each of R² and R⁴ is H. In another aspect n₅=2, n₉=3, X³ is

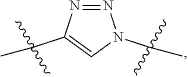 [SMILES: *c1cn(*)nn1]

R¹⁴ is H, R¹⁵ is H, R¹⁶ is OH, R¹⁷ is OH, and R¹⁸ is Cl.

In another aspect the compound as described herein R¹ is OH, R⁴ is

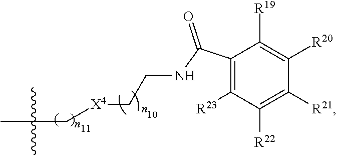

and each of R² and R³ is H. In another aspect n₁₀=1, n₁₁=0, X⁴ is a bond, R¹⁹ is Cl, R²⁰ is OH, R²¹ is OH, R²² is H, and R²³ is H.

In one aspect of the compound as described herein R¹ is OH, R⁴ is

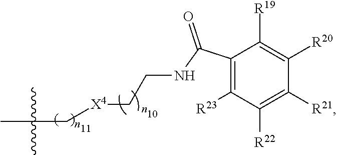

and each of R² and R³ is H. In another aspect n₁₀=1, n₁₁=3, X⁴ is

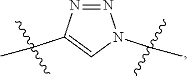 [SMILES: *c1cn(*)nn1]

R¹⁹ is H, R²⁰ is H, R²¹ is OH, R²² is OH, and R²³ is Cl.

In one embodiment the compounds of the present disclosure selected from the group consisting of:

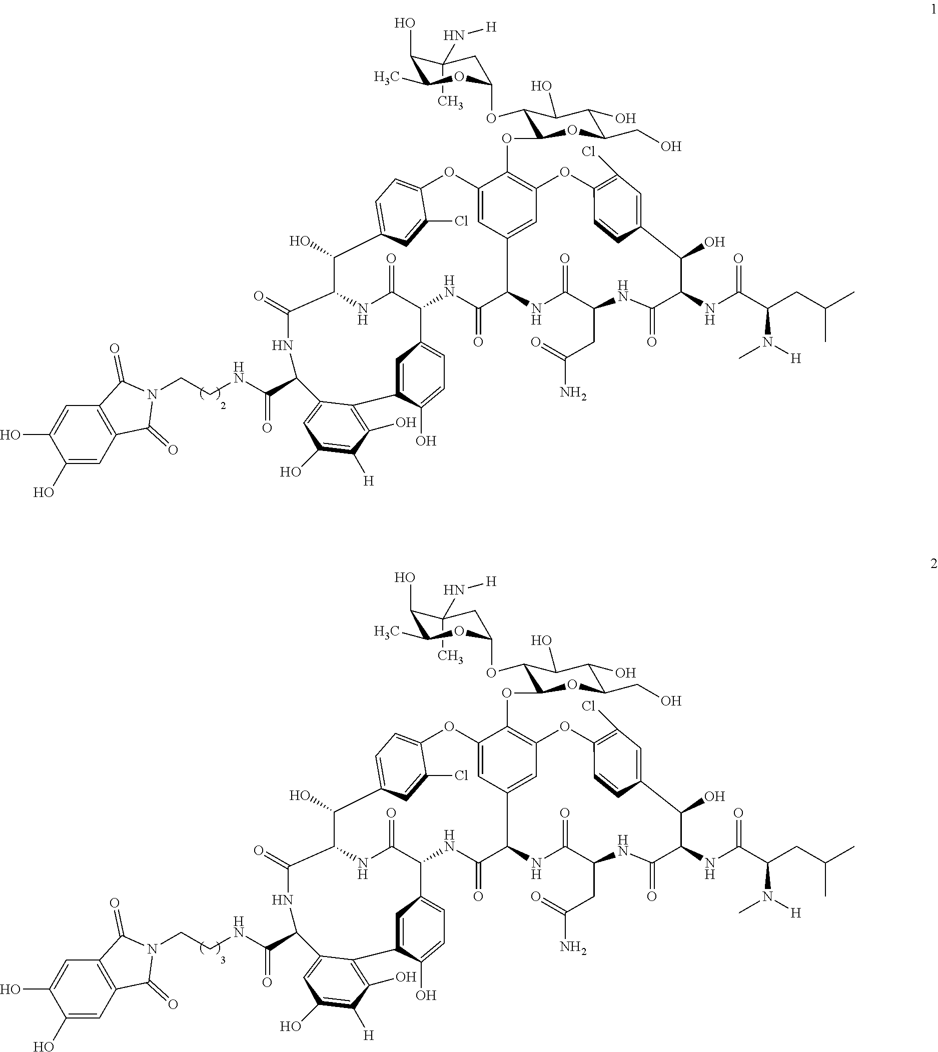

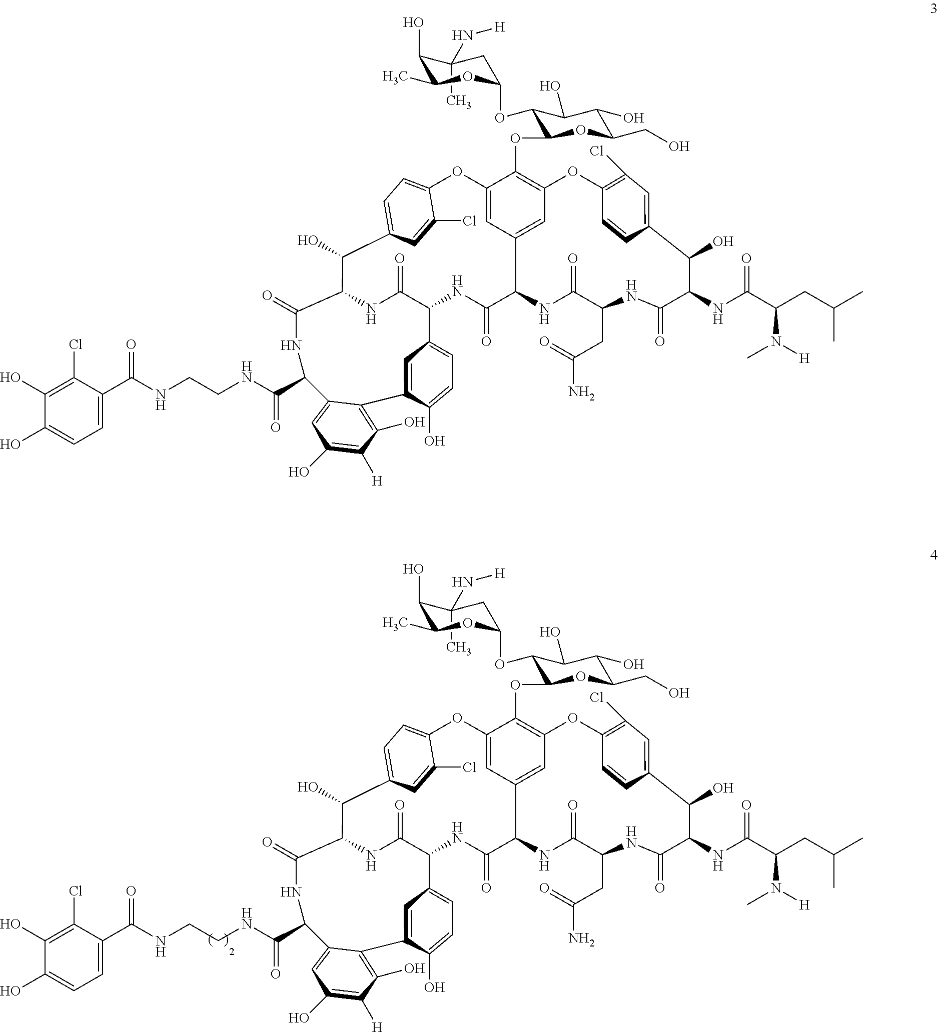

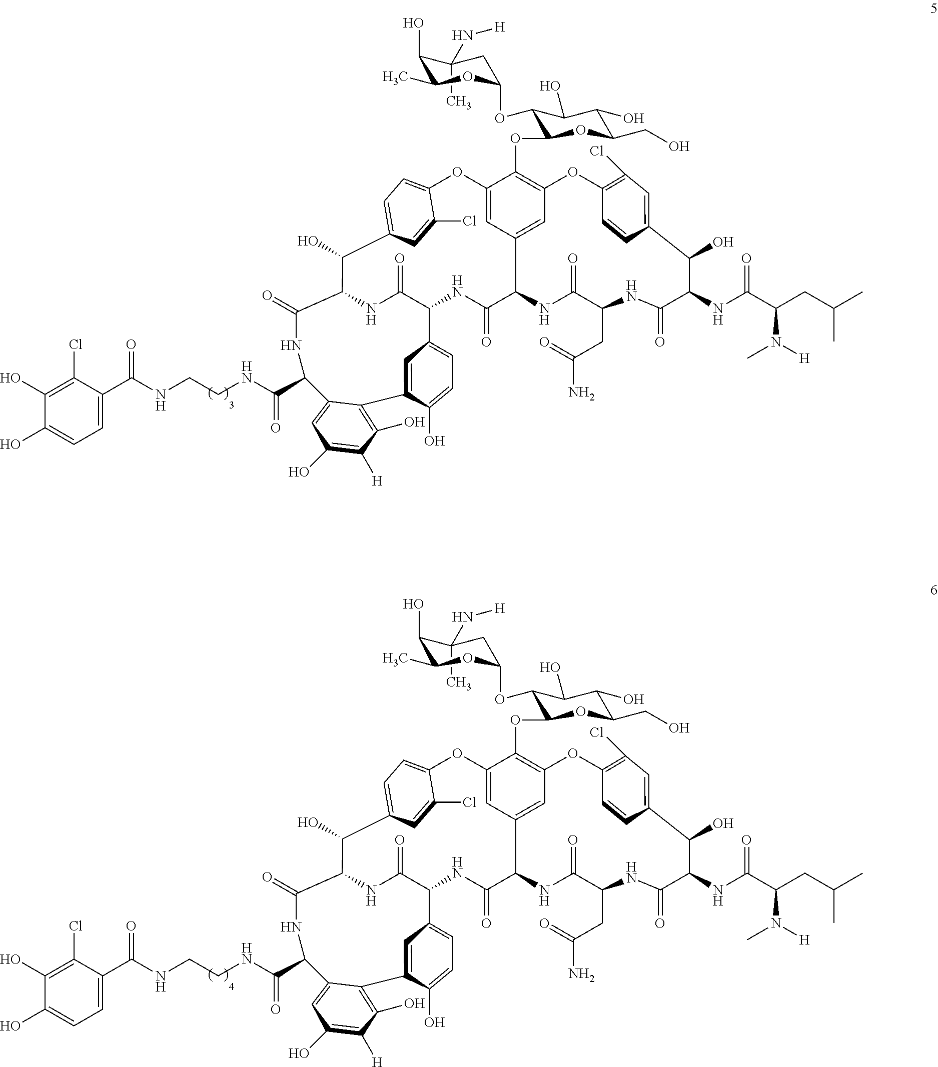

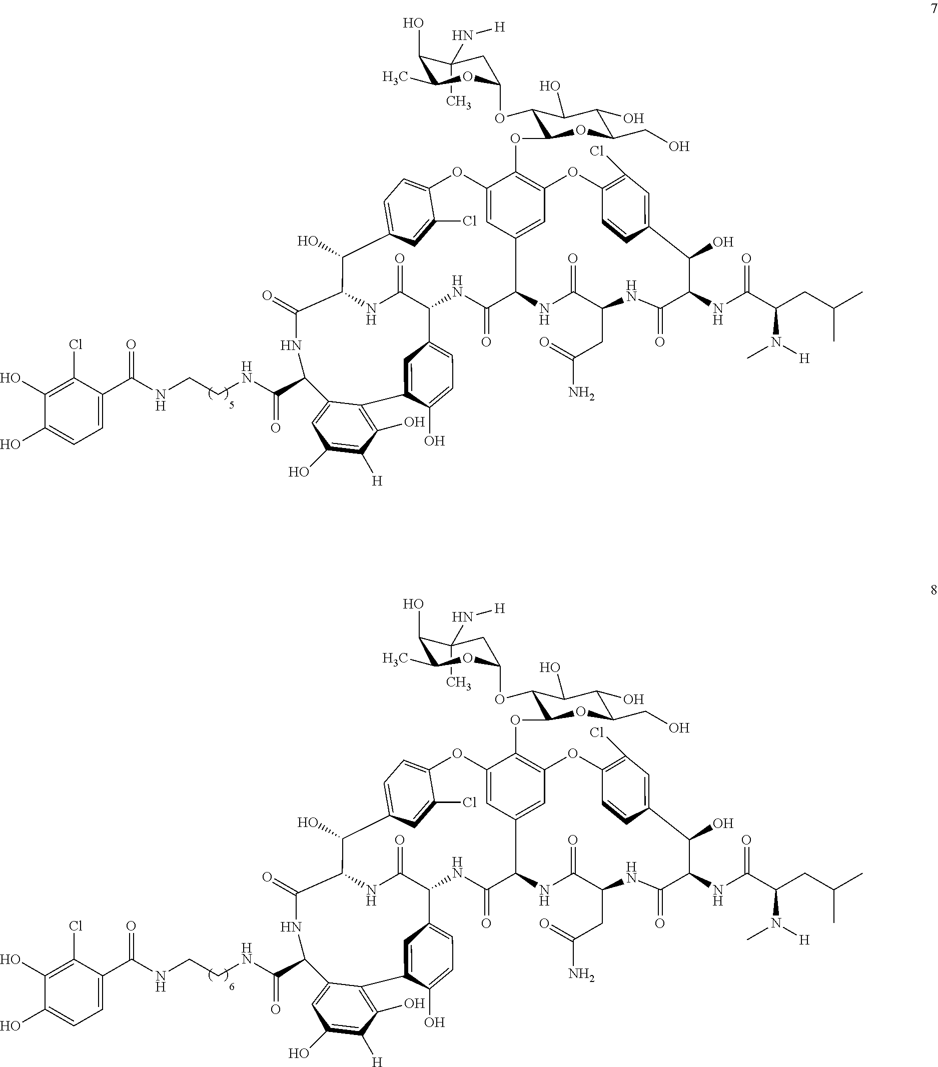

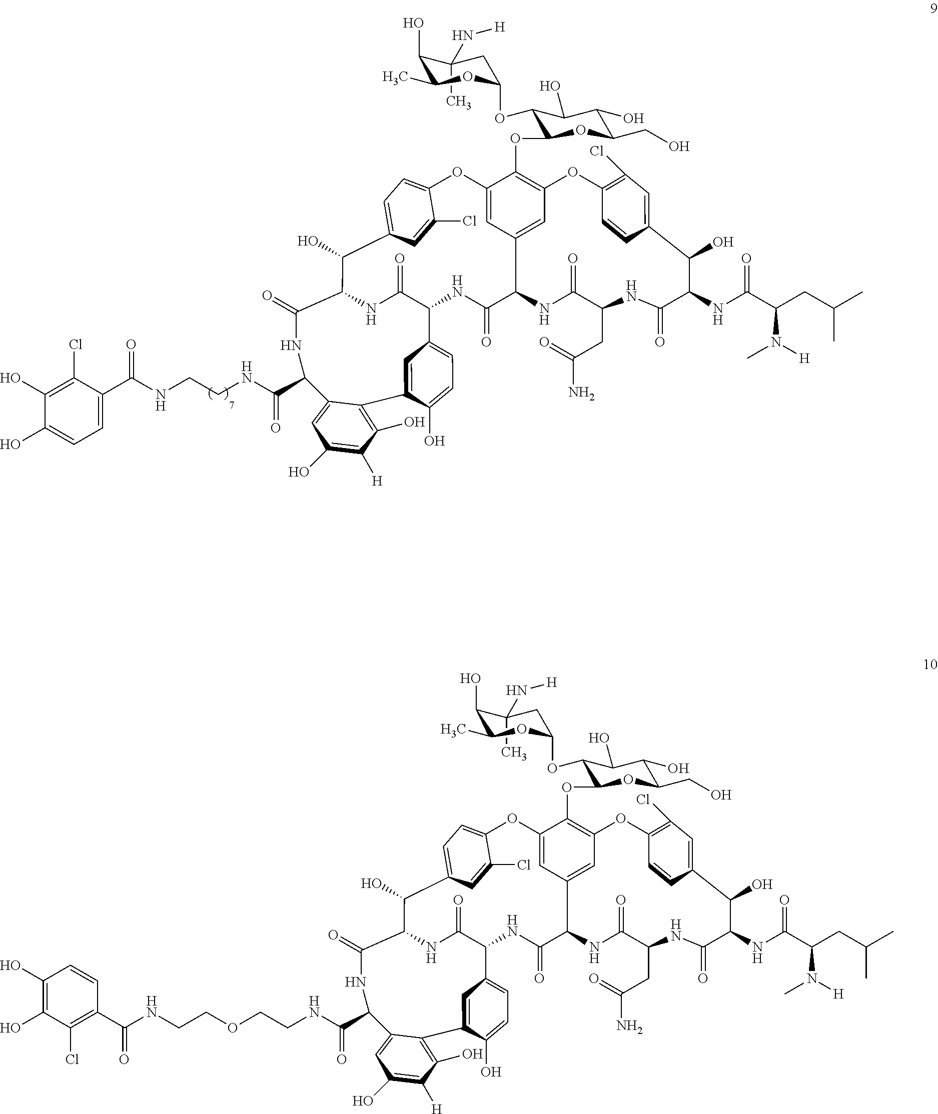

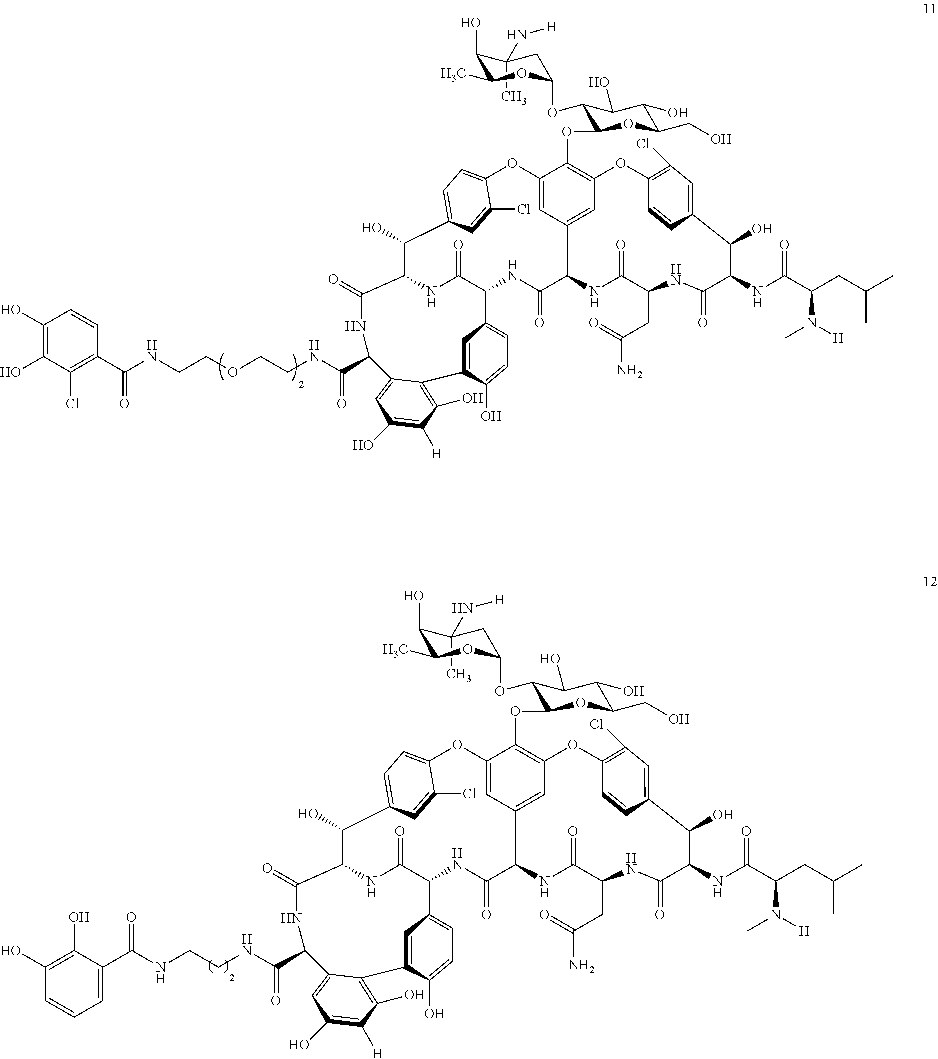

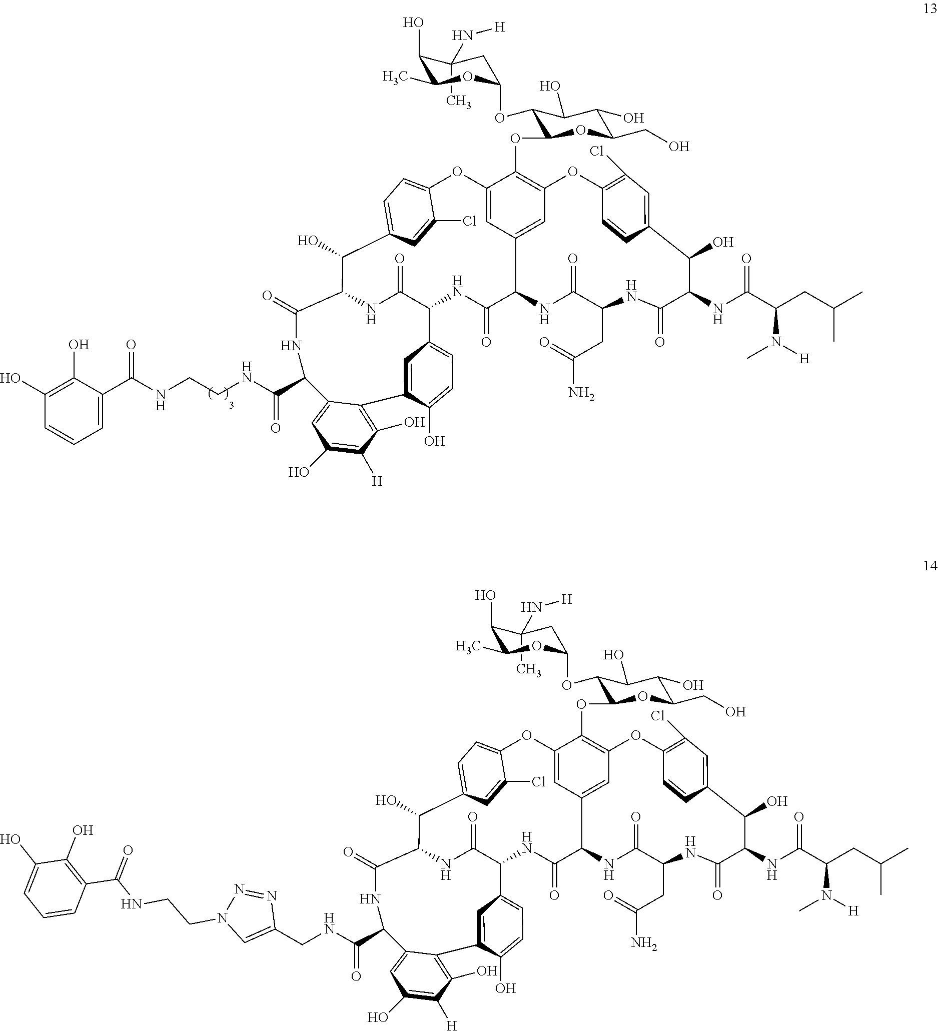

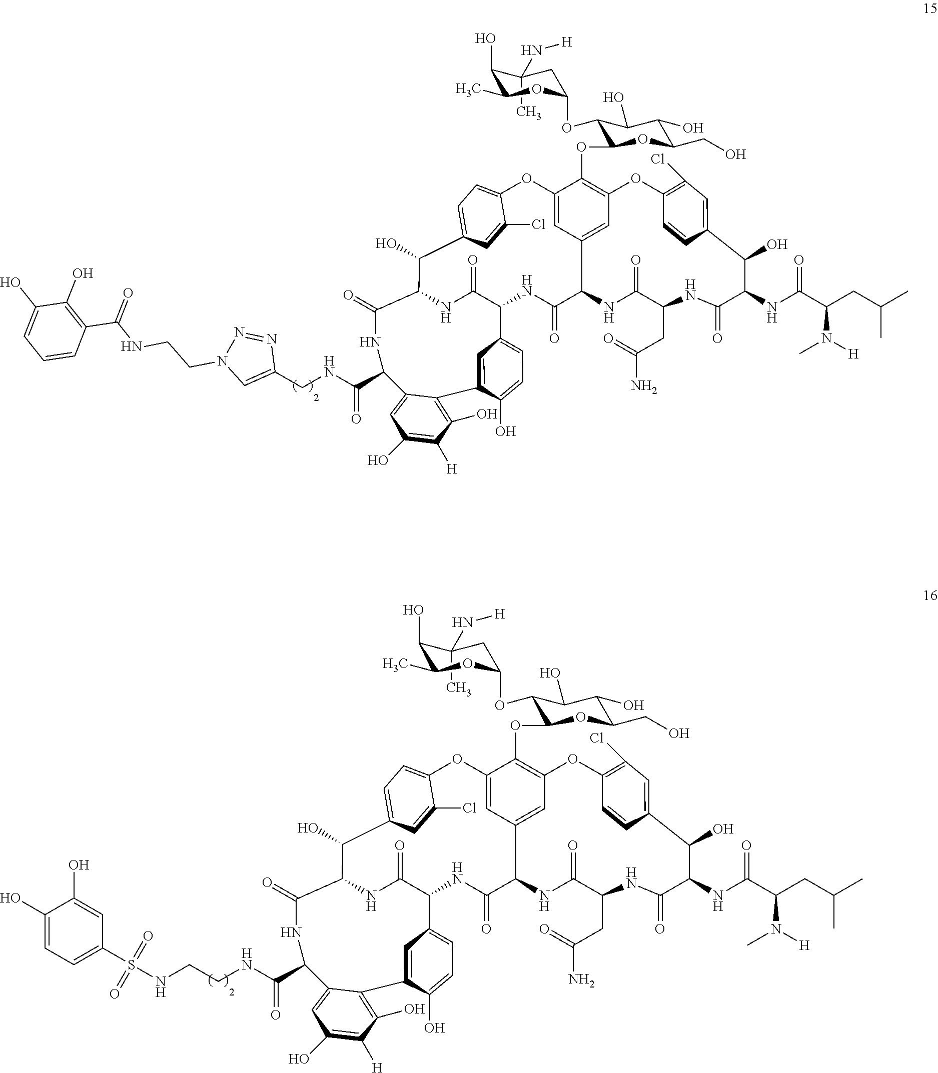

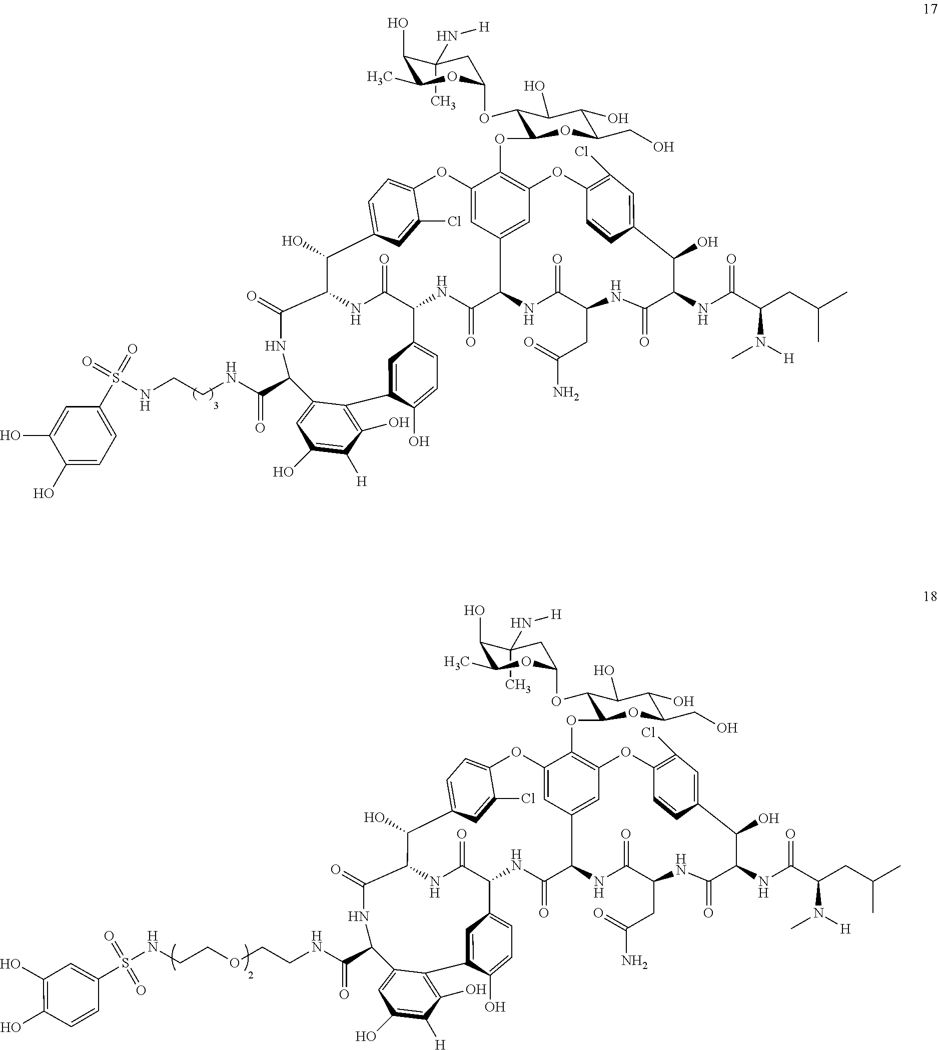

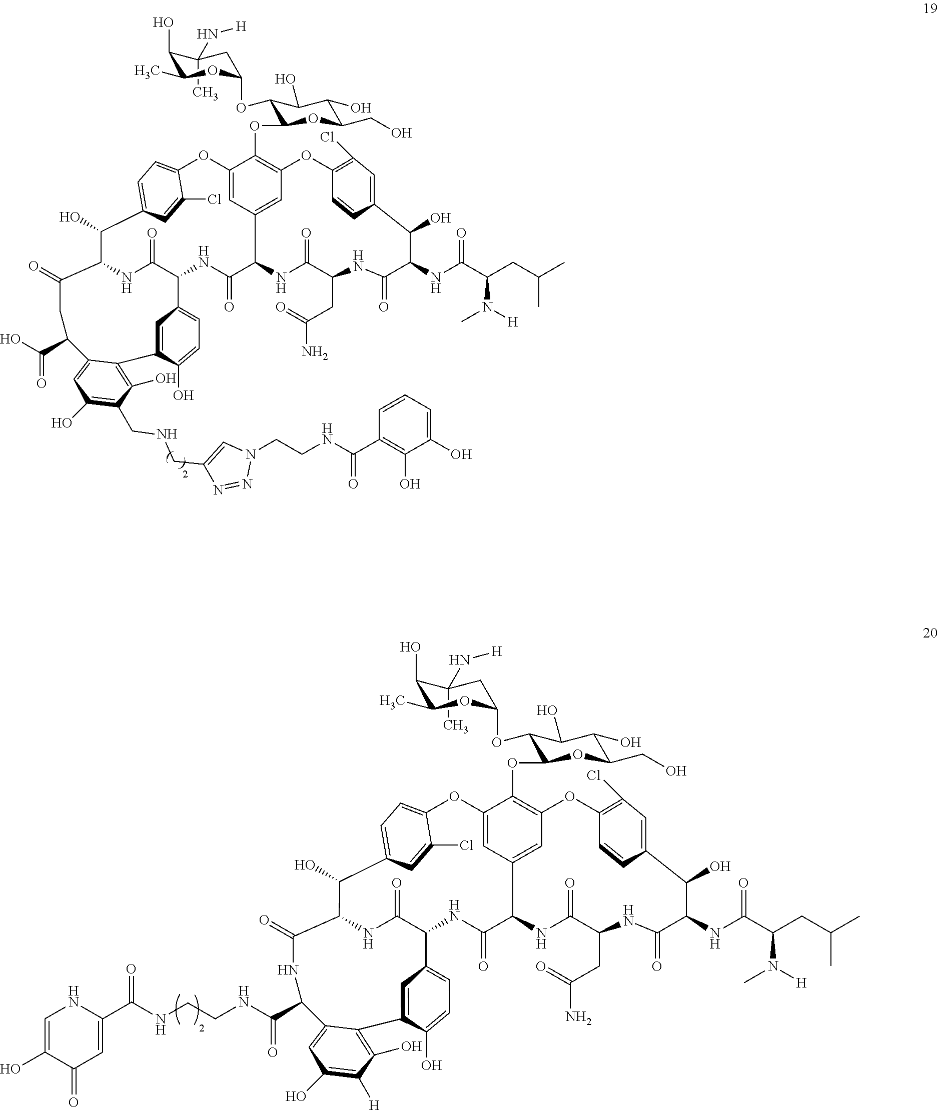

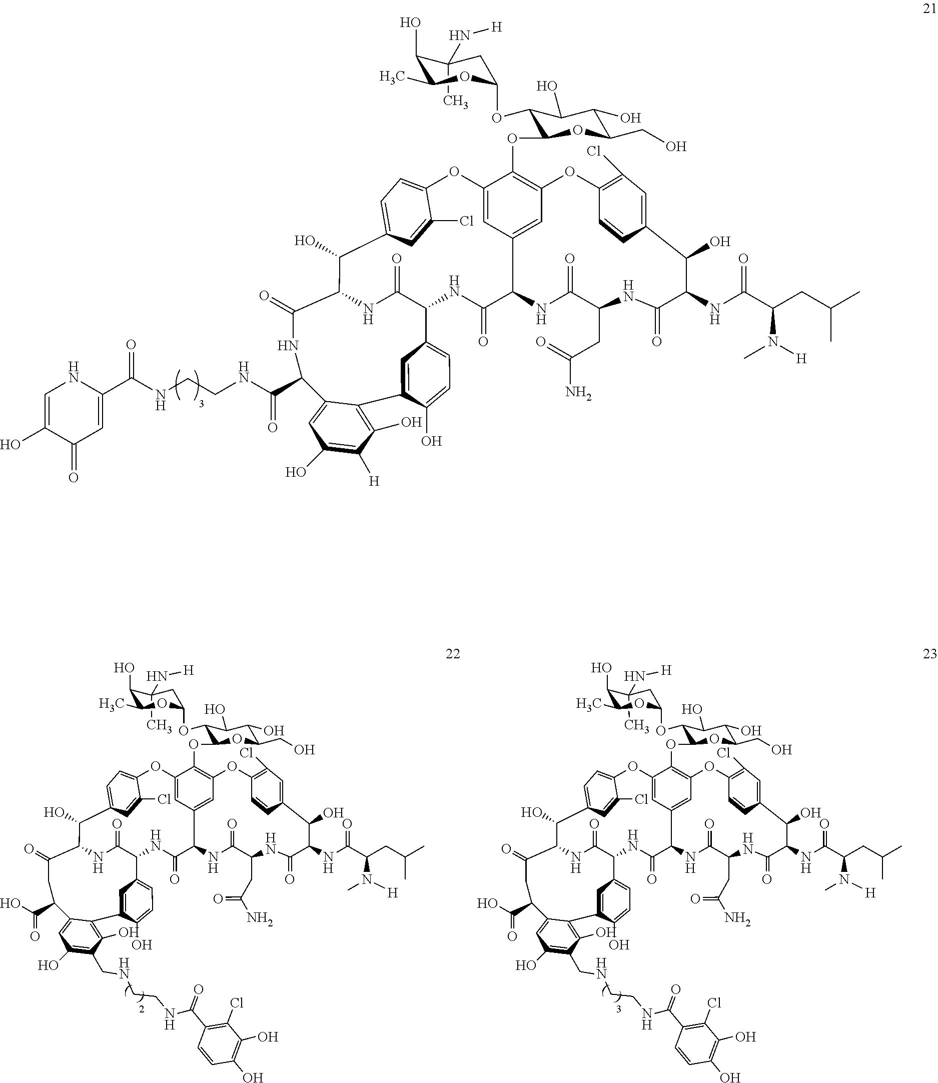

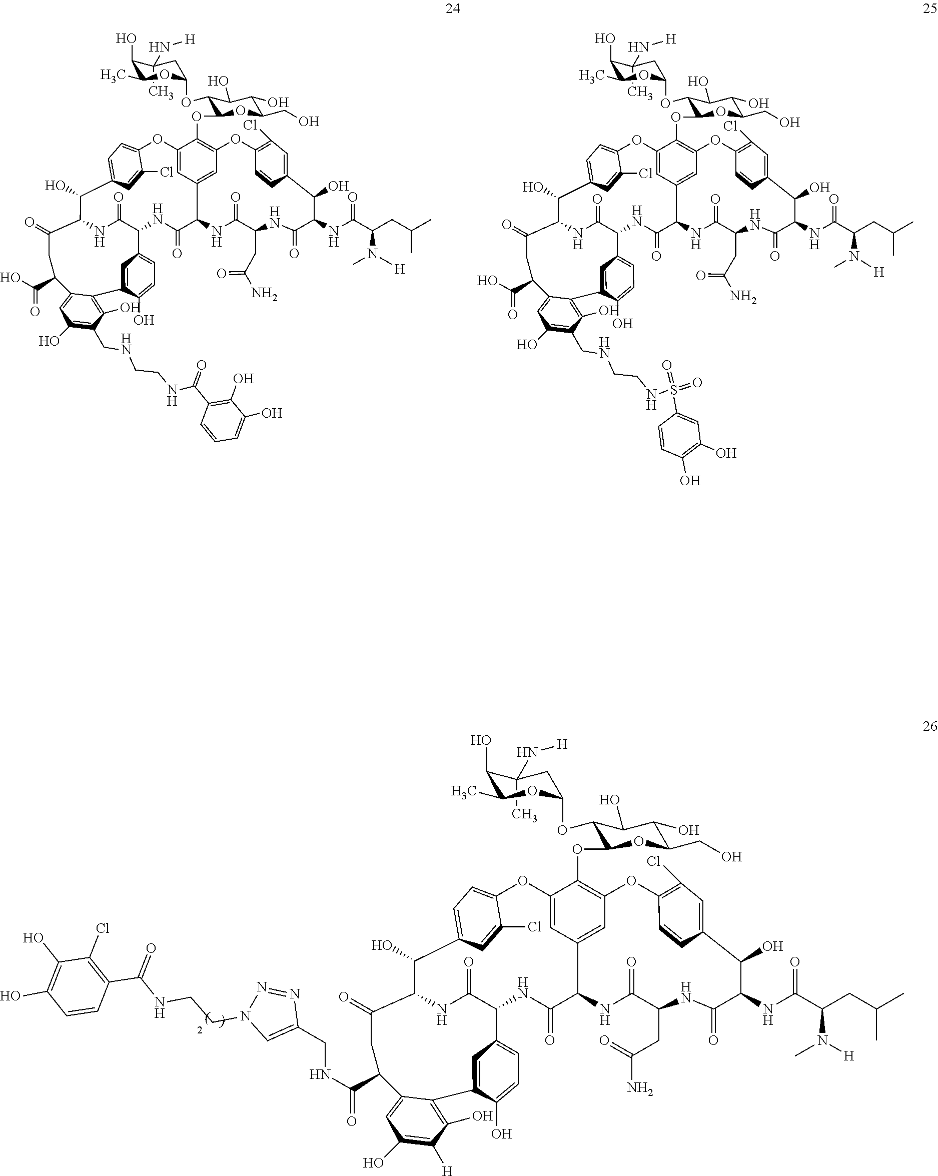

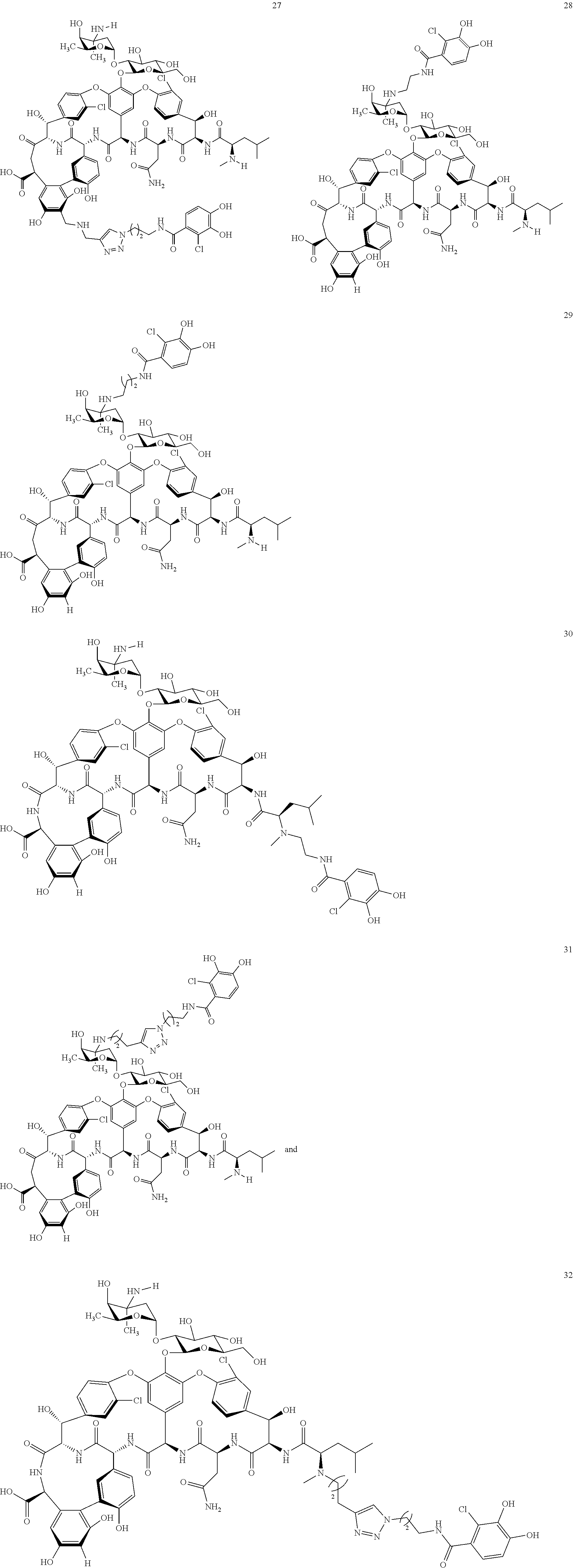

or a pharmaceutically acceptable salt thereof.

The following definitions are meant to clarify, but not limit, the teams defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C_{x-y} alkyl,” which refers to an alkyl group, as herein defined, containing a specified number of carbon atoms ranging from x-y. Similar terminology will apply for other preferred terms and ranges as well. Thus, for example, C₁₋₄ alkyl represents a straight or branched chain hydrocarbon containing one to four carbon atoms.

As used herein, the term “alkyl” alone or in combination with any other term, refers to a straight or branched chain hydrocarbon. Examples of “alkyl” as used herein include, but are not limited to, methyl, ethyl, propyl, isopropyl, isobutyl, n-butyl, tert-butyl, sec-butyl, iso-pentyl, n-pentyl, n-hexyl, and the like.

As used herein, the term “aryl” refers to a single benzene ring, fused, bridged, or spirocyclic benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein the term “halogen” refers to fluorine, chlorine, bromine, or iodine.

### Pharmaceutically Acceptable Salts and Compositions

As discussed herein, the disclosure provides compounds that are antibiotics, and thus the present compounds are useful for the treatment of diseases, disorders, and conditions including, but not limited to killing a bacterium or inhibiting, reducing, or preventing the growth thereof, comprising contact of said bacterium with a compound as defined herein. Accordingly, in another aspect of the disclosure, pharmaceutically acceptable compositions are provided, wherein these compositions comprise any of the compounds as described herein, and optionally comprise a pharmaceutically acceptable carrier, adjuvant, or vehicle. In certain embodiments, these compositions optionally further comprise one or more additional therapeutic agents.

As used herein, the term “pharmaceutically acceptable salt” refers to those salts which are, within the scope of sound medical judgement, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this disclosure that, upon administration to a recipient, is capable of providing, either directly or indirectly, a compound of this disclosure

It will also be appreciated that certain compounds of disclosure may exist in free form for treatment, or where appropriate, as a pharmaceutically acceptable derivative thereof. According to the disclosure, a pharmaceutically acceptable derivative includes, but is not limited to, pharmaceutically acceptable salts, esters, salts of such esters, or any other adduct or derivative which upon administration to a subject in need is capable of providing, directly or indirectly, a compound as otherwise described herein, or a metabolite or residue thereof.

Typically, but not absolutely, the salts of the present disclosure are pharmaceutically acceptable salts. Salts encompassed within the term “pharmaceutically acceptable salts” refer to non-toxic salts of the compounds of this disclosure. Salts of the compound of the present disclosure may comprise acid addition salts. Representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this disclosure and these should be considered to form a further aspect of the disclosure.

Also included are salts of amino acids such as arginate and the like, and salts of organic acids like glucuronic or galactunoric acids and the like (see, for example, Berge, S. M. et al, “Pharmaceutical Salts”, J. Pharm. Sci., 1977, 66:1-19). Certain specific compounds of the present disclosure contain both basic and acidic functionalities that allow the compounds to be converted into either base or acid addition salts.

“Salt thereof” refers to a compound formed when the hydrogen of an acid is replaced by a cation, such as a metal cation or an organic cation and the like. Preferably, the salt is a pharmaceutically-acceptable salt, although this is not required for salts of intermediate compounds which are not intended for administration to a patient.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that may readily undergo chemical changes under physiological conditions to provide the compounds of the present disclosure. Additionally, prodrugs may be converted to the compounds of the present disclosure by chemical or biochemical methods in an ex vivo environment.

In one aspect, the disclosure features a pharmaceutical composition comprising the compound of the disclosure and a pharmaceutically acceptable carrier.

In another aspect, the disclosure features a pharmaceutical composition comprising a therapeutically effective amount of the compound or a pharmaceutically acceptable salt thereof of the compounds of formula (I) and one or more pharmaceutically acceptable carriers or vehicles.

### Uses of Compounds and Pharmaceutically Acceptable Salts and Compositions

The present disclosure provides compounds that kill a bacterium or inhibit, reduce, or prevent the growth thereof, comprising contact of said bacterium with a compound as defined herein. Bacterial infection refers to bacteria residing and/or replicating in the body of a subject. Thus, the term covers any disease, disorder, pathology, symptom, clinical condition or syndrome in which bacteria act as actiological agents or in which infection with one or more strains of bacteria may be implicated, detected, or involved.

As used herein, the term “bacterial infection” is used to define a condition in which a subject is infected with a bacterium. The infection may be symptomatic or asymptomatic. In the latter case, the subject may be identified as infected on the basis of various tests, including for example biochemical tests, serological tests, microbiological culture and/or microscopy.

One embodiment of this disclosure relates to a method of controlling, treating or reducing the advancement, severity, or effects of a bacterial infection in a patient, comprising administering to said patient a therapeutically effective amount of a compound having the formula (I) or a pharmaceutically acceptable salt thereof.

The term “patient” as used herein, means the human or animal (in the case of an animal, more typically a mammal) subject. The patient may be one that is in need of the compositions or methods described herein. “In need of” may mean that the patient has or is diagnosed as having an infection e g. a bacterial infection, or that the patient is at risk of contracting an infection due to an injury, a medical, or surgical procedure, or microbial exposure, or could be in a position that could subject the patient to such exposure.

“Treating” or “treatment” as used herein refers to the treating or treatment of a disease or medical condition (such as a viral, bacterial, or fungal infection or other infectious diseases, as well as autoimmune or inflammatory conditions) in a patient, such as a mammal (particularly a human or a companion animal) which includes ameliorating the disease or medical condition, i.e., eliminating or causing regression of the disease or medical condition in a patient; suppressing the disease or medical condition, i.e., slowing or arresting the development of the disease or medical condition in a patient; or alleviating the symptoms of the disease or medical condition in a patient.

Additionally, the terms “treatment” or “treating” refers to an intervention (e.g. the administration of an agent to a subject) which prevents or delays the onset or progression of a disease or reduces (or eradicates) its incidence within a treated population. In this case, the term treatment may be used synonymously with the term “prophylaxis”.

According to another embodiment, the disclosure provides a method of decreasing or inhibiting bacterial quantity in a biological sample. This method comprises contacting said biological sample with a compound of formula (I) or a pharmaceutically acceptable salt thereof.

The term “biological sample”, as used herein, includes cell cultures or extracts thereof; biopsied material obtained from a mammal or extracts thereof; and blood, saliva, urine, feces, semen, tears, or other body fluids or extracts thereof. The term “biological sample” also includes living organisms, in which case “contacting a compound of this disclosure with a biological sample” is synonymous with the term “administrating said compound for composition comprising said compound to a mammal”.

The compounds of this disclosure, or pharmaceutical salts thereof, may be formulated into pharmaceutical compositions for administration to animals or humans. Thus, another embodiment described herein are pharmaceutical compositions effective to treat or prevent a bacterial infection may comprise the compounds of Formula (I) in an amount sufficient to measurably decrease bacterial quantity and a pharmaceutically acceptable carrier, adjuvant, or vehicle. The term “measurably decrease bacterial quantity”, as used herein means a measurable change in the number of bacteria between a sample containing said any one of the compounds described herein and a sample containing no compound.

According to another embodiment, the methods of the present disclosure are useful to treat patients in the veterinarian field including, but not limited to, zoo, laboratory, human companion, and farm animals including primates, rodents, reptiles and birds. Examples of said animals include, but are not limited to, guinea pigs, hamsters, gerbils, rat, mice, rabbits, dogs, cats, horses, pigs, sheep, cows, goats, deer, rhesus monkeys, monkeys, tamarinds, apes, baboons, gorillas, chimpanzees, orangutans, gibbons, ostriches, chickens, turkeys, ducks, and geese.

According to another embodiment, the present disclosure provides a method of decreasing or inhibiting Acinetobacter baumannii and Escherichia coli bacterial quantity in a biological sample comprising contacting said biological sample with the compound of formula (I), or a pharmaceutically acceptable salt thereof.

The pharmaceutical compositions and methods of the present disclosure may be useful for controlling bacterial infections in vivo. Examples of bacterial organisms that may be controlled by the compositions and methods of this disclosure include, but are not limited to the following organisms: Staphylococcus aureus, Acinetobacter baumannii, Pseudomonas aeruginosa, Klebsiella pneumonia, Escherichia coli, Neisseria gonorrhoeae.

Without intending to be bound by any particular theory, it is believed that the compounds provided herein are bacteriostatic or bacteriocidal in nature. The terms “bacteriostatic” and “bacteriocidal” are terms of art used to define the ability to prevent (or reduce the rate of) bacterial growth and to mediate (directly or indirectly) the cellular destruction of bacterial cells, respectively. The terms are not mutually exclusive, and many agents exert both bacteriostatic and bacteriocidal effects (in some cases in a dose-specific or target-specific manner). Compounds contemplated by the disclosure include, but are not limited to, the exemplary compounds provided herein and salts thereof.

For example, compounds of this disclosure may act as potent antibacterials and this activity may be further confirmed in minimum inhibitory concentration assays. Accordingly, the compounds provided herein are useful in pharmaceutical compositions, methods for the treatment of bacterial infections, and as controls in assays for the identification of antibacterial compounds.

The term “minimum inhibitory concentration” or “MIC” defines the lowest concentration of a test compound that is needed to inhibit growth of a bacterial isolate in vitro. A common method for determining the MIC of an antibiotic is to prepare several tubes containing serial dilutions of the test compound that are then inoculated with the bacterial isolate of interest. Following incubation at appropriate atmosphere and temperature, the MIC of an antibiotic can be determined from the tube with the lowest concentration that shows no turbidity.

### Compositions for Treatment

In another aspect, the present disclosure provides compositions that display antibacterial activity. Generally, the compositions for antibacterials in humans and animals comprise a pharmaceutically acceptable excipient or diluent and a compound having formula formula (I).

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self emulsifications as described in U.S. Pat. No. 6,451,339, hard or soft capsules, or syrups or elixirs. Compositions intended for oral use may be prepared according to any method known to the art for the manufacture of pharmaceutical compositions. Such compositions may contain one or more agents selected from sweetening agents, flavoring agents, coloring agents and preserving agents in order to provide pharmaceutically elegant and palatable preparations. Tablets contain the active ingredient in admixture with other non-toxic pharmaceutically acceptable excipients which are suitable for the manufacture of tablets. These excipients may be, for example, inert diluents such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated enterically or otherwise by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions may be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxypropylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecacthyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil in water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain one or more of a demulcent, a preservative, a flavoring agent, and coloring agent. Oral solutions can be prepared in combination with, for example, cyclodextrin, PEG, and surfactants.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non toxic parenterally acceptable diluent or solvent, for example as a solution in 1,3 butane diol. Among the acceptable vehicles and solvents that may be employed are water, Ringer's solution and isotonic sodium chloride solution. In addition, sterile, axed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions may be prepared by mixing the drug with a suitable non-irritating excipient, which is solid at ordinary temperatures but liquid at the rectal temperature and will therefore melt in the rectum to release the drug. Such materials are cocoa butter and polyethylene glycols. Additionally, the compounds can be administered via ocular delivery by means of solutions or ointments. Still further, transdermal delivery of the subject compounds can be accomplished by means of iontophoretic patches and the like.

For topical use, creams, ointments, jellies, solutions or suspensions containing the compounds of the present disclosure are employed. As used herein, topical application is also meant to include the use of mouth washes and gargles.

The pharmaceutical compositions and methods of the present disclosure may further comprise other therapeutically active compounds as noted herein, such as those applied in the treatment of the above mentioned pathological conditions.

In one embodiment, the present disclosure provides a composition consisting of a pharmaceutically acceptable carrier and a compound of the disclosure.

### Manufacture of Medicaments

In one aspect, the disclosure provides the use of a compound or pharmaceutical composition described herein for the manufacture of a medicament for use in the treatment of a bacterial infectious disease. In another aspect, the bacterial infection may be from Gram-negative or Gram-positive bacteria.

In yet another aspect, the disclosure provides the use of a compound or pharmaceutical composition described herein for the manufacture of a medicament for use in treating or lessening the severity in a subject with a bacterial infection.

### Administration of Pharmaceutically Acceptable Salts and Compositions.

In still other aspects, the present disclosure provides methods directed to the treatment of bacterial infections, comprising administering any of the compounds or composition of the present disclosure to a patient in need thereof. The method finds broad application in the treatment of any Gram-positive or Gram-negative bacterial infection or disease. The term Gram-positive and Gram-negative bacterium is a term of art defining a particular class of bacteria that are grouped together on the basis of certain cell wall staining characteristics.

In one aspect, the bacteria comprises a Gram-positive bacteria or Gram-negative bacteria. Exemplary Gram-negative bacteria include, but not limited to, Acinetobacter bauumannii, Eschericia coli, Klebsiella pneumonia, Haemophilus influenza, Helicobacter pylori, Bordatella pertussis, Pseudomonas aeruginosa, Neisseria gonorrhoeae, or Chlamydia trachomatis. Example Gram positive bacteria include, but not limited to, Staphylococcus aureus, Group A streptococcus, Mycobacterium tuberculosis, Bacillus coagulans, Listeria monoctyogenes, Clostridium botulinum, or Lactobacillus acidophilus. In one aspect, the Gram-negative bacteria comprises Acinetobacter baumannii or Escherichia coli.

For use in therapy, therapeutically effective amounts of a compound of formula (I), as well as salts or solvates thereof, may be administered as the raw chemical. Additionally, the active ingredient may be presented as a pharmaceutical composition.

In certain embodiments of the disclosure an “effective amount” of the compound, a pharmaceutically acceptable salt thereof or pharmaceutically acceptable composition is that amount effective for killing a bacterium or inhibiting, reducing, or preventing the growth thereof, comprising contact of said bacterium with a compound as defined herein.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

Accordingly, the disclosure further provides pharmaceutical compositions that include effective amounts of one or more compounds of the formula (I), or a salt or solvate thereof, and one or more pharmaceutically acceptable carriers, diluents, or excipients. The compound of formula (I) or a salt or solvate thereof, are as herein described. The carrier(s), diluent(s), or excipient(s) must be acceptable, in the sense of being compatible with the other ingredients of the composition and not deleterious to the recipient of the pharmaceutical composition.

The therapeutically effective amount of a compound of the present disclosure will depend upon a number of factors. For example, the species, age, and weight of the recipient, the precise condition requiring treatment and its severity, the nature of the composition, and the route of administration are all factors to be considered. The therapeutically effective amount may be at the discretion of the attendant physician or veterinarian. Regardless, an effective amount of a compound of formula (I) for the treatment of humans suffering from bacterial infections or disease, generally, may be in the range of 0.1 to 100 mg/kg body weight of recipient (mammal) per day. The effective amount may also be in the range of 0.1 to 20 mg/kg body weight per day. Thus, for a 70 kg adult mammal one example of an actual amount per day may be from 10 mg to 2000 mg. This amount may be given in a single dose per day or in a number (such as two, three, four, five, or more) of sub-doses per day such that the total daily dose is the same. An effective amount of a salt or solvate thereof, may be determined as a proportion of the effective amount of the compound of formula (I) per se. Similar dosages should be appropriate for treatment of the other conditions referred to herein. Pharmaceutical compositions may be presented in unit dose forms containing a predetermined amount of active ingredient per unit dose. Such a unit may contain, as a non-limiting example, 1 mg to 2 g of a compound of the formula (I), depending on the condition being treated, the route of administration, and the age, weight, and condition of the patient. Preferred unit dosage compositions are those containing a daily dose or sub-dose, as herein above recited, or an appropriate fraction thereof, of an active ingredient. Such pharmaceutical compositions may be prepared by any of the methods well known in the pharmacy art.

A compound of the present disclosure or a salt or solvate thereof, may be employed alone or in combination with other therapeutic agents. Exemplary additional therapeutic agents include, but are not limited to, one or more cold, cough, allergy, nasal decongestant, antitussive, expectorant, antihistamine, stimulant, sedative, anti-inflammatory, antibiotic, anti-viral, anti-asthmatic, anti-migraine, hypnotic, narcotic analgesic, or narcotic antagonist active pharmaceutical ingredients, or further combinations thereof.

As used herein, the term “combination”, as applied to two or more compounds and/or agents (also referred to herein as the components), is intended to define material in which the two or more compounds/agents are associated. The terms “combined” and “combining” in this context are to be interpreted accordingly.

As used herein, the term “combination therapy” is intended to define therapies which comprise the use of a combination of two or more compounds/agents. Thus, references to “combination therapy”, “combinations” and the use of compounds/agents “in combination” in this application may refer to compounds/agents that are administered as part of the same overall treatment regimen. As such, the posology of each of the two or more compounds/agents may differ: each may be administered at the same time or at different times. It will therefore be appreciated that the compounds/agents of the combination may be administered sequentially (e.g. before or after) or simultaneously, either in the same pharmaceutical formulation (i.e. together), or in different pharmaceutical formulations (i.e. separately). Simultaneously in the same formulation is as a unitary formulation whereas simultaneously in different pharmaceutical formulations is non-unitary. The posologist of each of the two or more compounds/agents in a combination therapy may also differ with respect to the route of administration.

The compound of formula (I) and the other pharmaceutically active agent(s) may be administered together or separately and, when administered separately, administration may occur simultaneously or sequentially, in any order. The amounts of the compound of formula (I) and other pharmaceutically active agent(s) and the relative timings of administration may be selected in order to achieve the desired combined therapeutic effect. The administration in combination of a compound of formula (I) or a salt or solvate thereof with other treatment agents may be in combination by administration concomitantly in: (1) a unitary pharmaceutical composition including a combination of compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds and compositions of the present disclosure can be combined with other compounds and compositions or treatment agents having related utilities to prevent and treat the condition or disease of interest, such as bacterial infections and diseases. Selection of the appropriate treatment agents for use in combination therapies can be made by one of ordinary skill in the art. The combination of therapeutic agents may act synergistically to effect the treatment or prevention of the various disorders. Using this approach, one may be able to achieve therapeutic efficacy with lower dosages of each agent, thus reducing the potential for adverse side effects.

The compounds of formula (I) may also be co-administered with other antibiotics to increase the effect of therapy or prophylaxis against various bacterial infections. When the compounds of this disclosure are administered in combination therapies with other agents, they may be administered sequentially or concurrently to the patient. Alternatively, pharmaceutical or prophylactic compositions according to this disclosure comprise a combination of a compound of formula (I) and another therapeutic or prophylactic agent. Exemplary additional therapeutic agents include, but are not limited to an antibiotic selected from a natural penicillin, a penicillinase-resistant penicillin, an antipseudomonal penicillin, an aminopenicillin, a first generation cephalosporin, a second generation cephalosporin, a third generation cephalosporin, a fourth generation cephalosporin, a carbapenem, a cephamycin, a quinolone, a fluoroquinolone, an aminoglycoside, a macrolide, a ketolide, a polymyxin, a tetracycline, a glycopeptide, a streptoGramin, an oxazolidinone, a rifamycin, or a sulfonamide.

Although probiotic therapy is suggested to improve immune system response in gut infected patients, intravenous immunoglobulin (J. Antimicrob. Chem., 2004, 53, 882-884), for example, may also be used to treat patients, particularly recurrent cases where any further antimicrobial treatment would further exacerbate gut flora disturbance. Thus, the compounds of the disclosure may be used adjunctively with various immunoglobulins.

In some embodiments, compounds of formula (I) may be used prophylactically to prevent a bacterial infection. In some embodiments, compounds of formula (I) may be used before, during or after a dental or surgical procedure to prevent opportunistic infections.

The term “prevent a bacterial infection” as used herein, unless otherwise indicated, means the prophylatic use of an antibiotic, such compounds of the present disclosure, to prevent a bacterial infection. Treatment with an antibiotic may be done prophylactically to prevent an infection caused by an organism that is susceptible to antibiotics of Formula (I). One general set of conditions where prophylactic treatment may be considered is when an individual is more vulnerable to infection due to, for example, weakened immunity, surgery, trauma, presence of an artificial device in the body (temporary or permanent), an anatomical defect, exposure to high levels of bacteria or possible exposure to a disease-causing pathogen. Examples of factors that could lead to weakened immunity include chemotherapy, radiation therapy, diabetes, advanced age, HIV infection, and transplantation. An example of an anatomical defect would be a defect in the heart valve that increases the risk of bacterial endocarditis. Examples of artificial devices include artificial joints, surgical pins, catheters, etc. Another set of situations where prophylactic use of an antibiotic may be appropriate would be to prevent the spread of a pathogen between individuals (direct or indirect).

One embodiment of the disclosure includes a method of treating a bacterial infection comprising administering to a subject an effective amount of any compound or a pharmaceutically acceptable salt thereof as described herein, or a composition as described herein. One aspect of the embodiment includes a method wherein the bacterial infection treated results from Gram-positive bacteria or Gram-negative bacteria. A further aspect of this embodiment includes a method wherein the bacterial infection treated results from Acinetobacter baumannii and Escherichia coli.

One embodiment of the disclosure includes a method of treating a bacterial infection comprising administering to a subject an effective amount of any compound or a pharmaceutically acceptable salt thereof as described herein, or a composition as described herein in combination with another treatment agent.

### EXAMPLES

### Chemistry

The synthesis of siderophore building blocks is depicted in Scheme 1. Five siderophore groups were chosen, categorized into two types, namely catecholate and 3-hydroxypiridone, respectively. In general, the amide bond with different aliphatic linkers or PEGylated linkers was formed via coupling reaction (i, iii, iv, vi), followed by removal of the protecting group with BBr₃ or HCl (ii, v). As for the azido-containing building blocks, the NH₂ functionality was converted to an azide using imidazole-1-sulfonyl azide (vii). For synthesis of aldehyde building blocks, the alcohol was oxidized to aldehyde with Dess-Martin periodinane (viii), followed by BBr₃ treatment to remove the methyl group on phenol (ii).

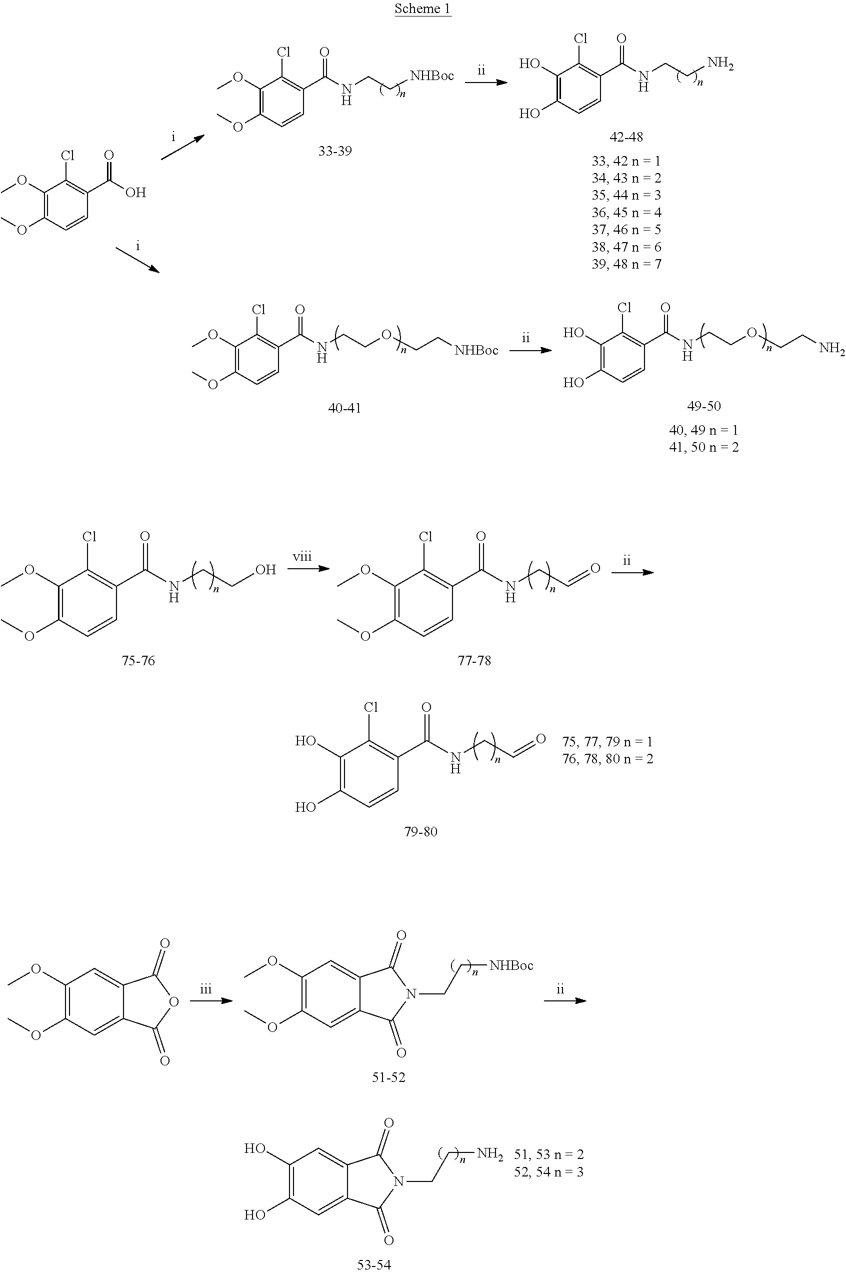

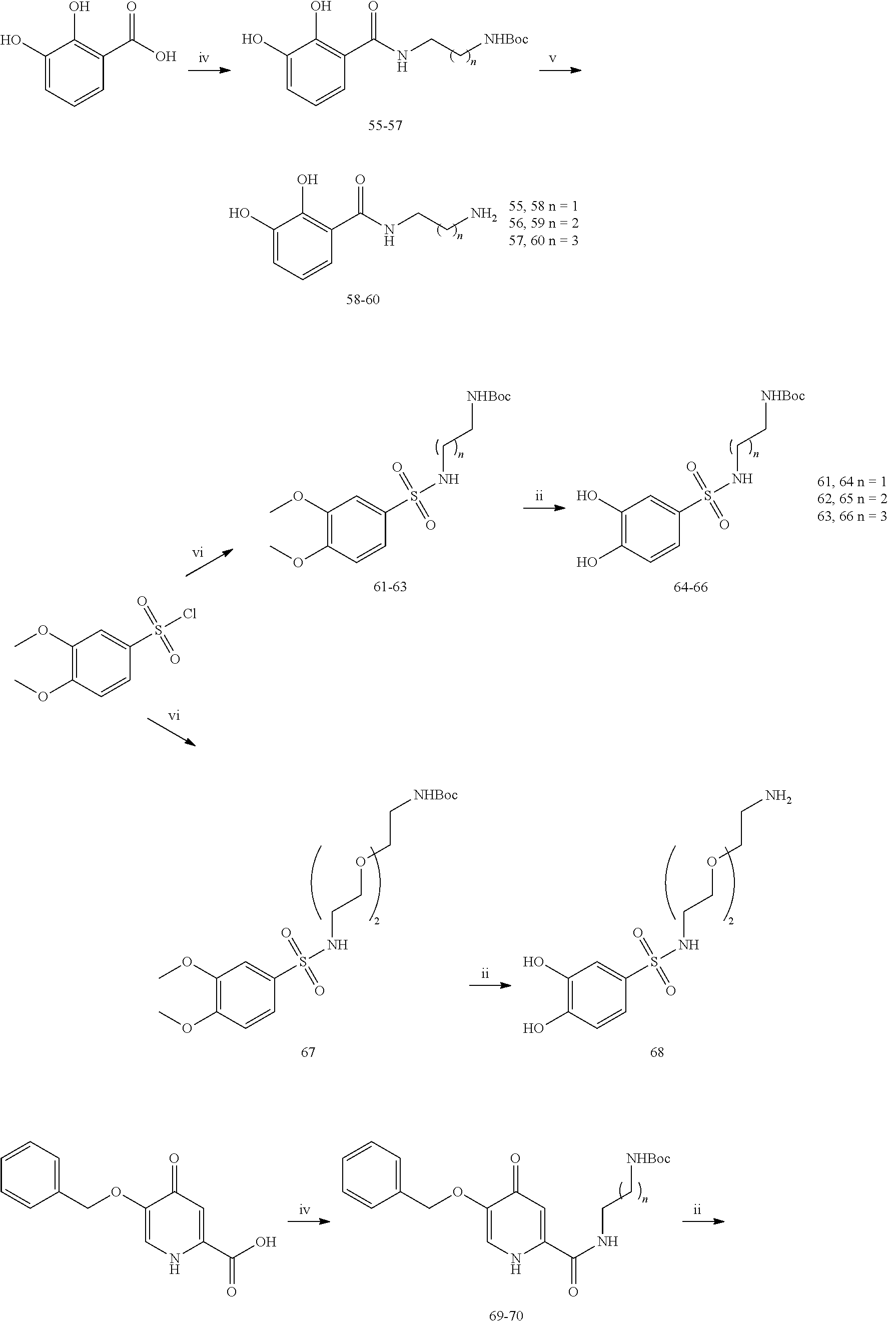

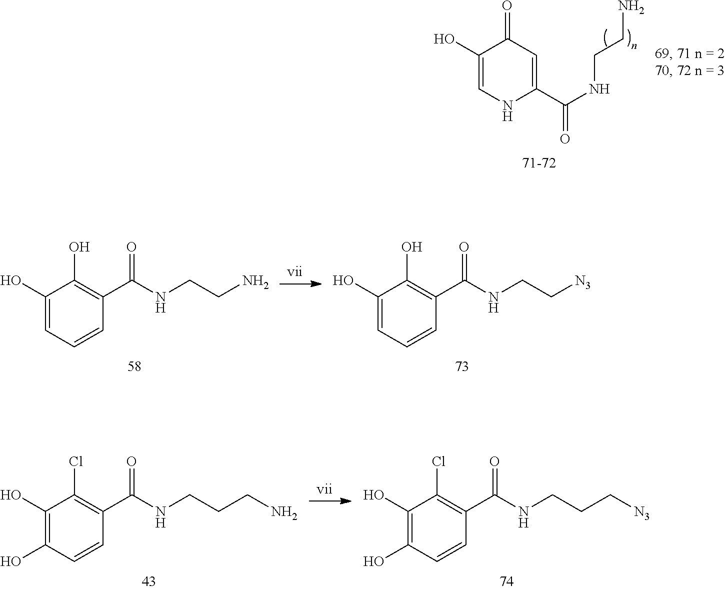

Scheme 1. Synthesis of siderophore building blocks. Reagents and Conditions: i) N-Boc-diamine, HATU, DIPEA, DCM; ii) BBr₃, DCM, −78° C., 30 min, then warmed to rt, 30 min; iii) N-Boc-diamine, toluene, microwave irradiation, 170° C., 10 min; iv) N-Boc-1,2-diaminoethane, HOBT, EDC-HCl, TEA, THF, rt, 24 h; v) 4 M HCl in dioxane, rt, 40 min; vi) N-Boc-diamine, DIPEA, DCM; vii) imidazole-1-sulfonyl azide HCl salt, CuSO₄·5H₂O, K₂CO₃, MeOH; viii) Dess-Martin periodinane, DCM, rt, 2 h.

The synthesis of target compounds 1-18, 20 and 21 is depicted in Scheme 2. Site-specific amide coupling was utilized for the C-terminus modification of vancomycin skeleton after obtaining the above amine-building blocks, which furnished the target vancomycin-siderophore conjugates and corresponding alkynyl intermediates.

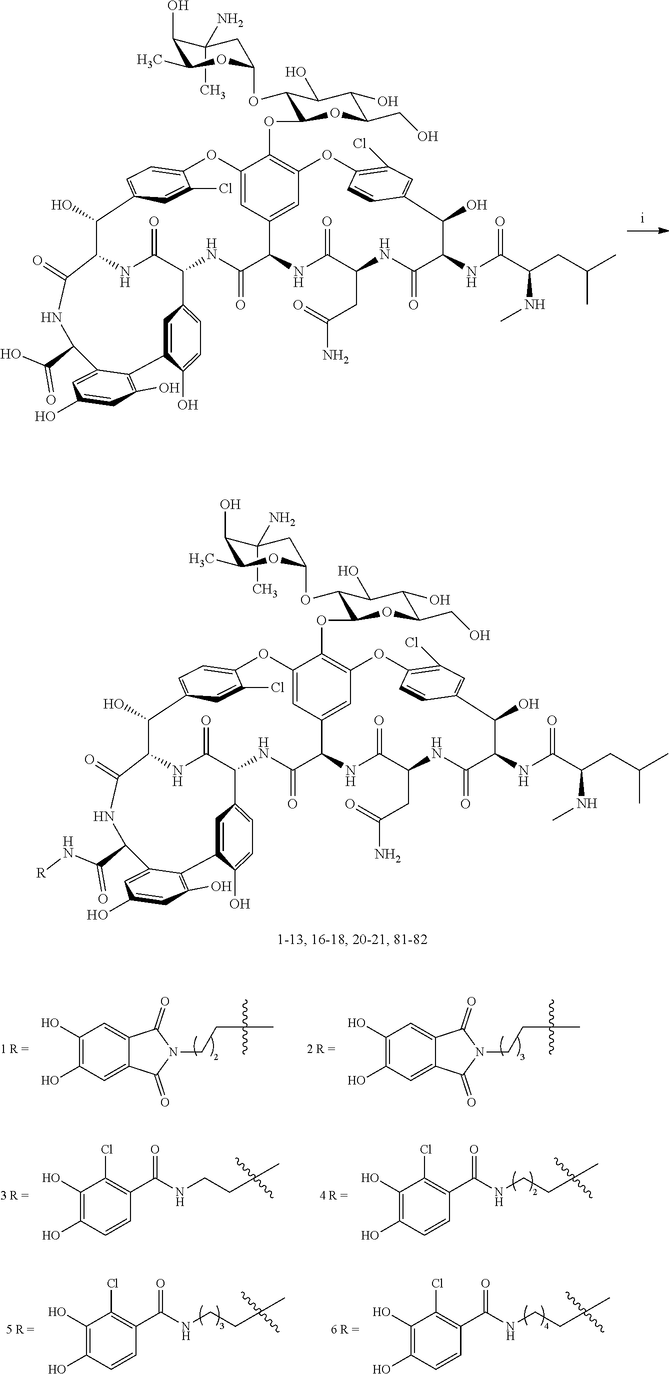

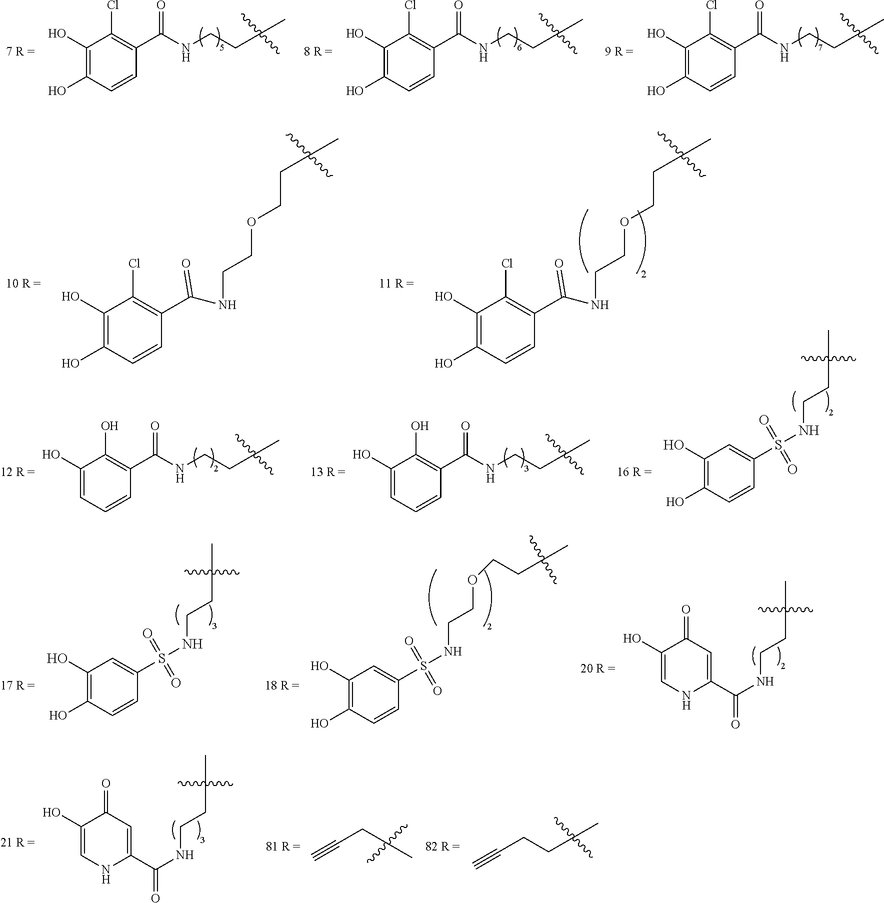

Scheme 2. Synthesis of targets 1-18, 20, 21 and alkynyl intermediates modified at C-terminus. Reagents and Conditions: i) HATU, DIPEA, DMF/DMSO, rt, 30 min to 2 h.

The synthesis of target compounds 22-25 is depicted in Scheme 3. The Mannich reaction was used to selectively modify the Resorcinol (R)-position to give the corresponding vancomycin-siderophore conjugates as well as the alkynyl-containing intermediates.

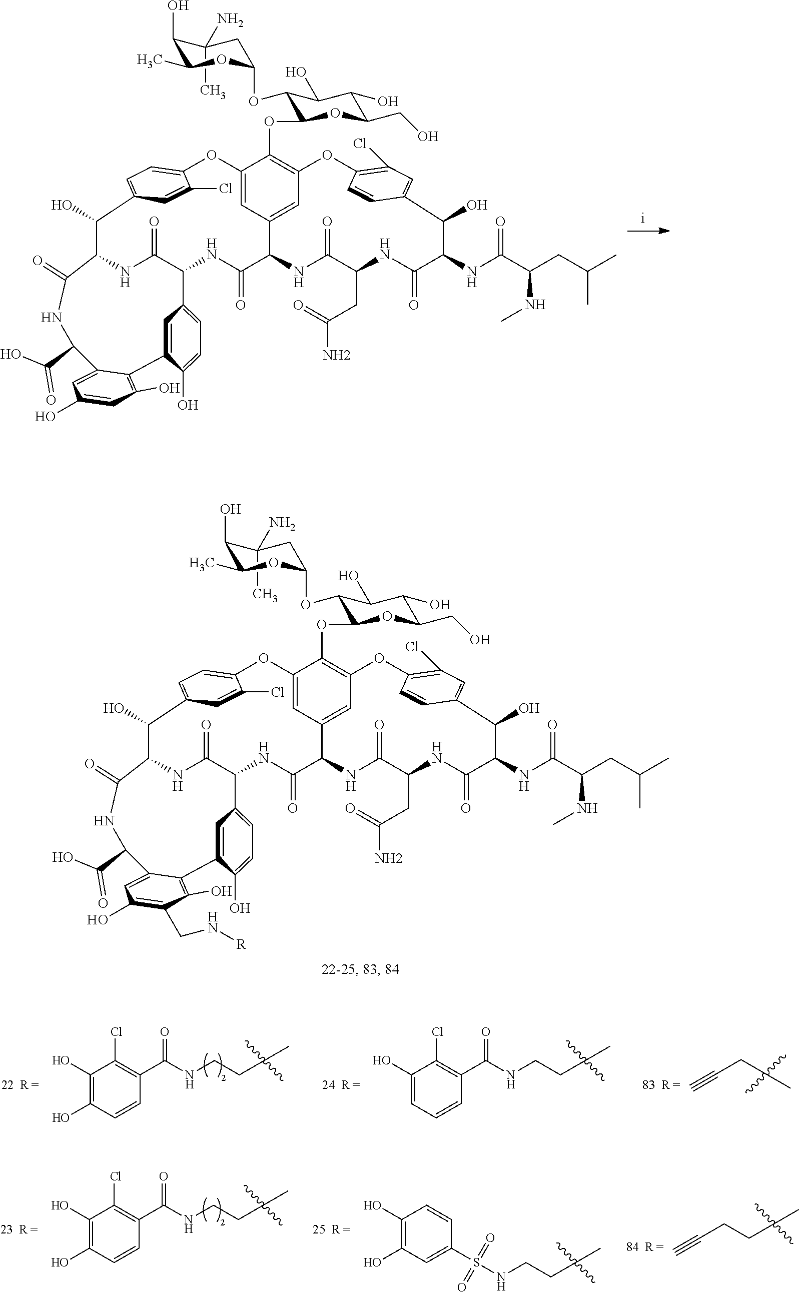

Scheme 3. Synthesis of targets 22-25 and alkynyl intermediates modified at R-position. Reagents and Conditions: i) HCHO, DIPEA, H₂O/CH₃CN, −10° C., 12 to 24 h.

The synthesis of target compounds 28-29 is depicted in Scheme 4. The specific vancosamine (V)-position modification was achieved by the 2-step reductive amination reaction, firstly formed imine of C—N double bond under basic conditions followed by reduction with NaCNBH₃ under acid conditions.

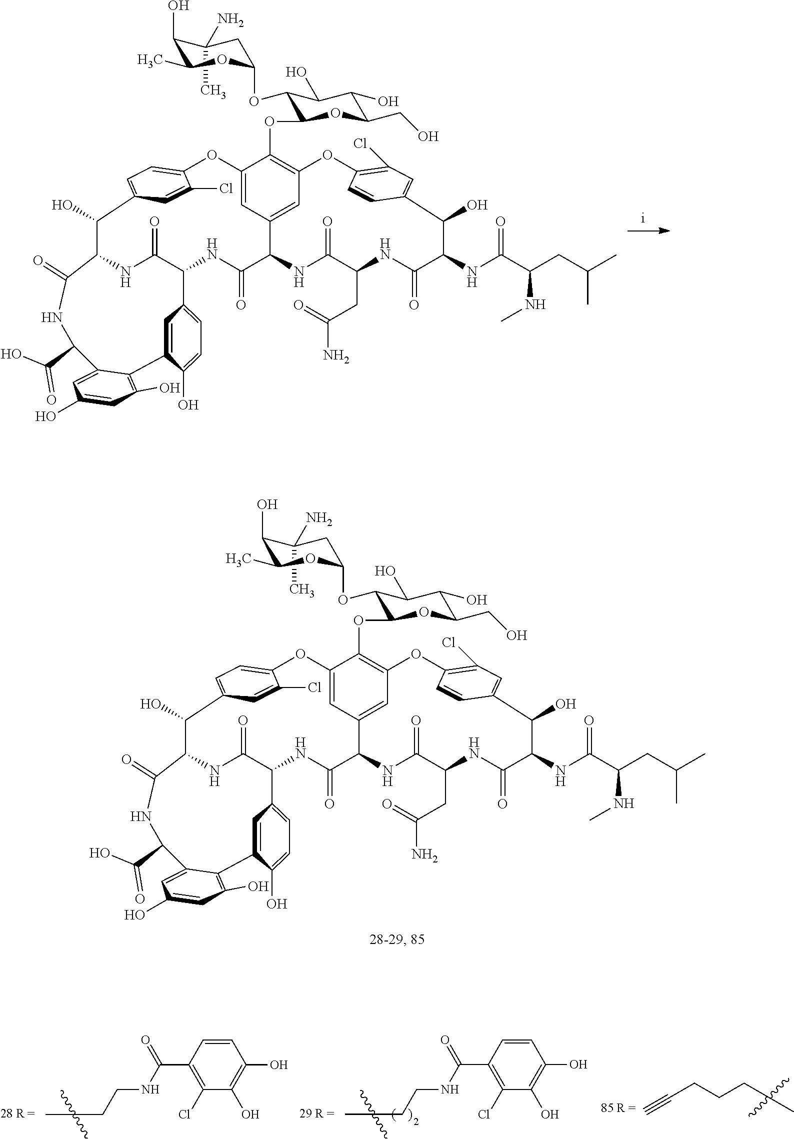

Scheme 4. Synthesis of targets 28-29 and alkynyl intermediates modified at V-position. Reagents and Conditions: i) (a) DIPEA, DMF, 50° C., 1 h; (b) NaCNBH₃, MeOH, TFA, DMF, rt, 3 h.

The synthesis of target compound 30 is depicted in Scheme 5. N-terminus was specifically modified by the aqueous acid reductive amination reaction to afford vancomycin-conjugate 30 and alkynyl intermediates modified at N-terminus.

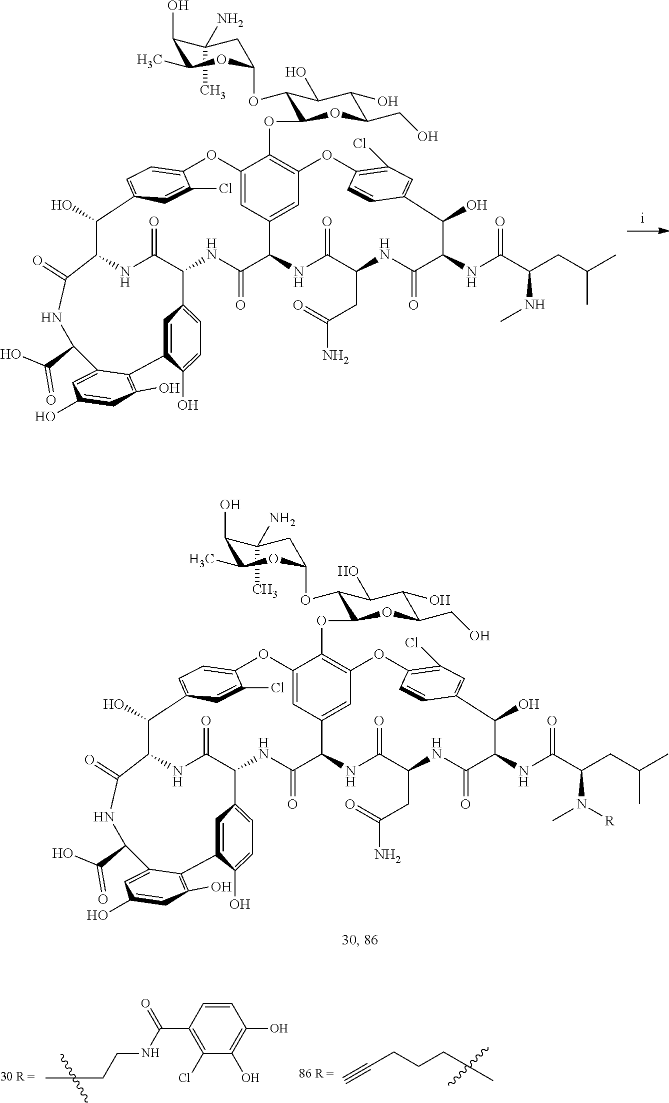

Scheme 5. Synthesis of target 30 and alkynyl intermediates modified at N-terminus. Reagents and Conditions: i) NaCNBH₃, AcOH—H₂O—CH₃CN (1:2:2), 70° C., 7 h.

The synthesis of target compounds 14, 15, 19, 26, 27, 31 and 32 is depicted in Scheme 6. Click reaction was applied to produce the triazole linkage to get the corresponding vancomycin-siderophore conjugates. The linker lengths and siderophore moieties varied for each target compound.

[symbol]

[symbol]

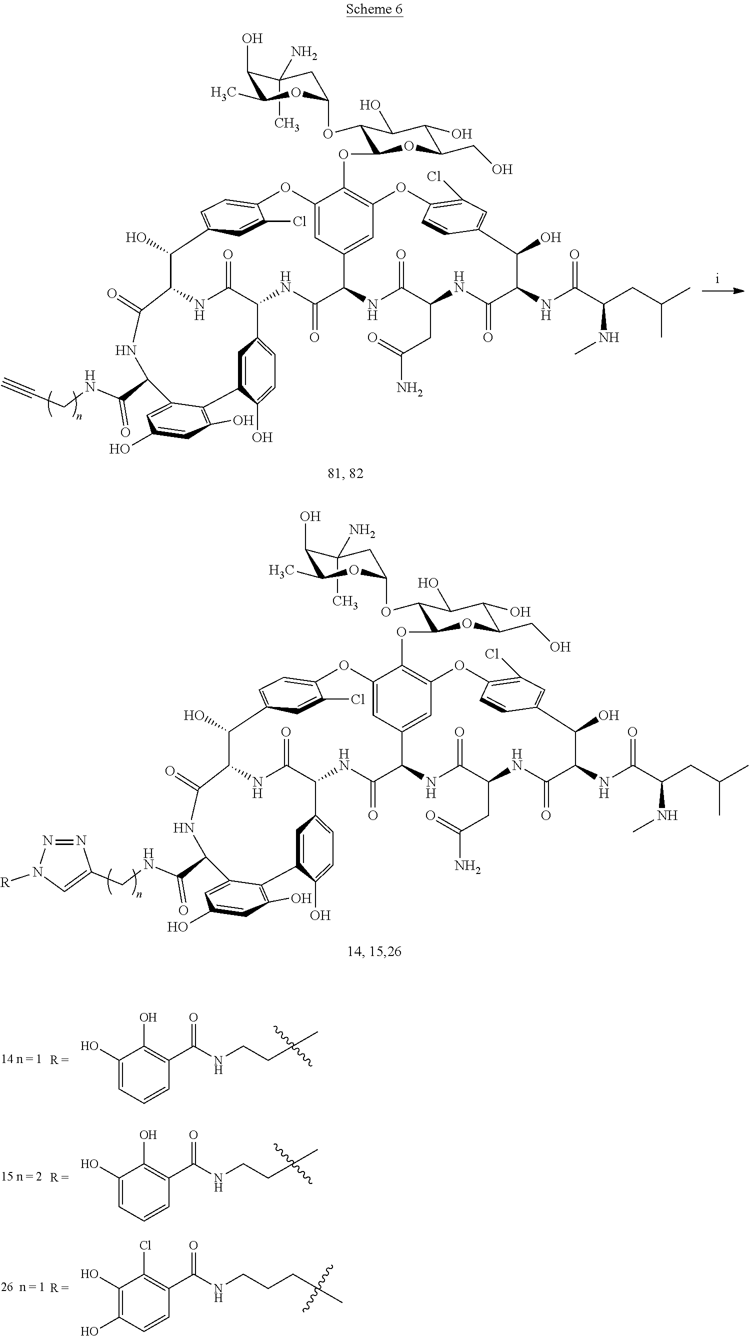

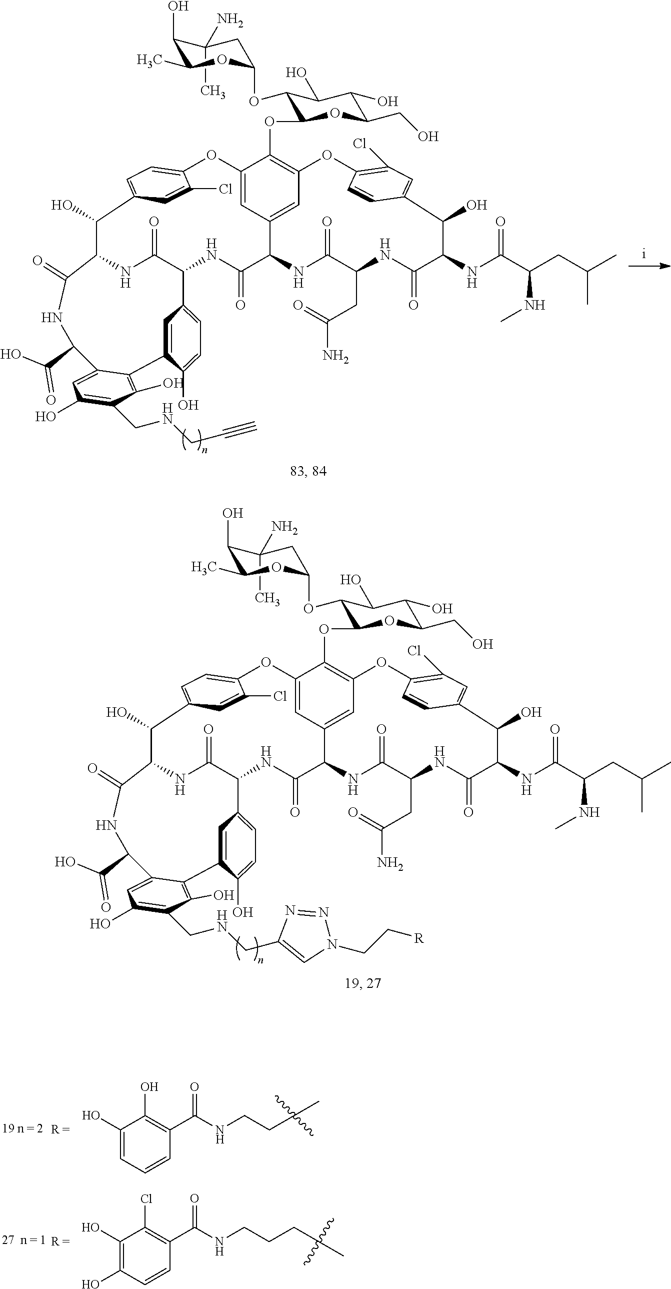

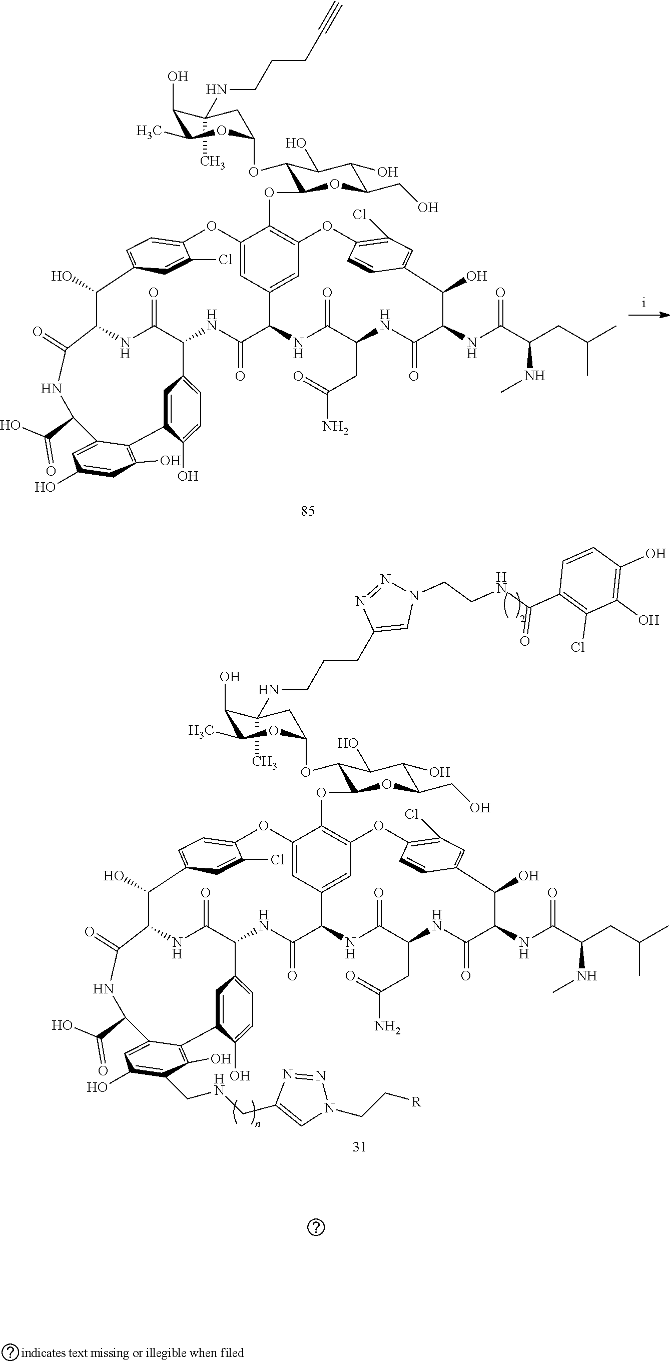

[symbol]

Scheme 6. Synthesis of targets 14, 15, 19, 26, 27, 31 and 32 with a triazole linkage. Reagents and Conditions: i) azido-building blocks, NaHCO₃, CuSO₅·5H₂O, sodium ascorbate, H₂O/t-BuOH, rt, 2 h.

### Experimental

All solvents and chemicals were reagent grade. Unless otherwise stated, all reagents and solvents were purchased from commercial vendors and used as received. Flash column chromatography was conducted on a Teledyne ISCO CombiFlash Rf system using prepacked silica gel columns to get the required intermediates or building blocks. Analytical RP-HPLC was used to monitor the reaction progress, which was performed on a VARIAN ProStar HPLC instrument (Photodiode Array Detector) with a C18 column (XBridge®, 5 μm, 4.6 mm×250 mm). The column was eluted with a gradient of 2→58.8% (or 2→90%) CH₃CN containing 0.1% TFA in 20 min (or 30 min) at a flow rate of 1 mL/min. Preparative RP-HPLC preparation was conducted on a VARIAN ProStar HPLC instrument with Photodiode Array Detector. The XBridge® Prep C18 5 μm OBDTM column (19×250 mm) was used with the elution method of a gradient of 2→58.8% (or 2→90%) CH₃CN containing 0.1% TFA in 20 min (or 30 min) at a flow rate of 7 mL/min.

Purity and characterization of compounds were established by a combination of NMR, mass spectrometry, TLC, and HPLC analyses. 1H and 13C NMR spectra were recorded on a Bruker Avance DPX-300 (300 MHz) spectrometer and were determined in CDCl₃, DMSO-d₆, or CD₃OD with tetramethylsilane (TMS) (0.00 ppm) or solvent peaks as the internal reference. 20 μL D₂O was added to exchange the active hydrogen of target products when the solvent was DMSO-d₆. Chemical shifts are reported in ppm relative to the reference signal and coupling constant (J) values are reported in hertz (Hz). High resolution mass spectra (HRMS) were obtained using Agilent 1290 Infinity UHPLC-6230 TOF mass spectrometer (ESI). Nominal mass spectra were obtained using an Agilent InfinityLab MSD single quadrupole mass spectrometer system (ESI). Thin-layer chromatography (TLC) was performed on EMD precoated silica gel 60 F254 plates, and spots were visualized with UV light or iodine staining.

All final compounds were greater than 95% pure as determined by HPLC on a Waters 2695 Separation Module equipped with a Waters 2996 Photodiode Array Detector and a Phenomenex Synergi 4 mm Hydro-RP 80A C18 250×4.6 mm column using a flow rate of 1 mL/min starting with 1 min at 5% solvent B, followed by a 15 min gradient of 5→95% solvent B, followed by 9 min at 95% solvent B (solvent A, water with 0.1% TFA; solvent B, acetonitrile with 0.1% TFA and 5% water; absorbance monitored at 280 nm).

### Example 1: Synthesis of Compounds 33-50 (Scheme 1)

To a mixture of 2-chloro-3,4-dimethoxybenzoic acid (650 mg, 3 mmol) and N-Boc-diamine (3.3 mmol) in DCM (20 mL) was added DIPEA (1 mL, 6 mmol) and HATU (1.5 g, 3.9 mmol) successively. This mixture was stirred at room temperature for 2 h until no progression was monitored by TLC. Then DCM (30 mL) was added, followed by the H₂O (50 mL) wash to remove the hydrophilic byproducts. The collected DCM layer was concentrated and purified by flash chromatography to obtain the corresponding intermediates 33-41. The deprotection was performed using BBr₃ in DCM. To a stirred solution of above intermediates (1 g) in 10 mL anhydrous DCM at −78° C. was added 3 equivalents of BBr₃ solution (1 M in DCM) dropwise. The mixture was stirred at −78° C. for 30 min. It was warmed to room temperature and stirred for another 30 min. After the completion of the reaction detected by TLC and LCMS, the reaction was quenched with MeOH (20 mL) at 0° C. The resulting mixture was concentrated. Ether was added to the residue and the solution left to stand for 48 h. If there was no crystal precipitated, vacuum drying was used to get the deprotected product. The siderophore building blocks 42-49 HBr salt were obtained as off-white solid or light-brown hygroscopic residue. The compound 50 was purified by RP-C18 Prep-HPLC to give a corresponding TFA salt.

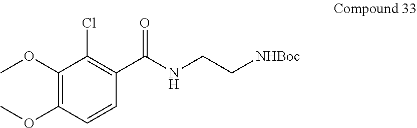 [SMILES: COc1ccc(C(=O)NCCNC(=O)OC(C)(C)C)c(Cl)c1OC]

¹H NMR (300 MHz, CDCl₃) δ 7.41 (d, J=8.7 Hz, 1H), 6.84 (d, J=8.7 Hz, 1H), 6.79 (s, 1H), 5.01 (s, 1H), 3.90 (s, 3H), 3.85 (s, 3H), 3.64-3.47 (m, 2H), 3.38 (q, J=5.9 Hz, 2H), 1.43 (s, 9H).

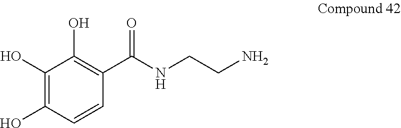 [SMILES: NCCNC(=O)c1ccc(O)c(O)c1O]

¹H NMR (300 MHz, CD₃OD) δ 6.96 (d, J=8.3 Hz, 1H), 6.77 (d, J=8.3 Hz, 1H), 3.62 (t, J=6.2 Hz, 2H), 3.16 (t, J=6.3 Hz, 2H).

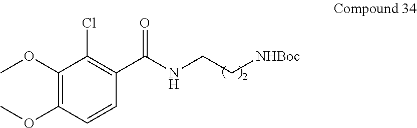

¹H NMR (300 MHz, CDCl₃) δ 7.41 (d, J=8.7 Hz, 1H), 6.86 (d, J=8.7 Hz, 1H), 6.77 (s, 1H), 5.02 (s, 1H), 3.90 (s, 3H), 3.86 (s, 3H), 3.51 (q, J=6.3 Hz, 2H), 3.24 (q, J=6.3 Hz, 2H), 1.82-1.67 (m, 2H), 1.43 (s, 9H).

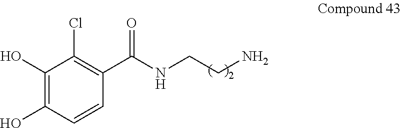

¹H NMR (300 MHz, DMSO-d₆) δ 8.31 (t, J=5.8 Hz, 1H), 7.84 (s, 3H), 6.79 (d, J=8.2 Hz, 1H), 6.74 (d, J=8.2 Hz, 1H), 3.26 (q, J=6.4 Hz, 2H), 2.86 (q, J=6.8 Hz, 2H), 1.79 (p, J=6.8 Hz, 2H).

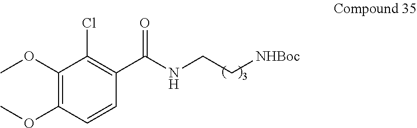

¹H NMR (300 MHz, CDCl₃) δ 7.45 (d, J=8.6 Hz, 1H), 6.86 (d, J=8.7 Hz, 1H), 6.45 (s, 1H), 4.66 (s, 1H), 3.90 (s, 3H), 3.85 (s, 3H), 3.47 (q, J=6.3 Hz, 2H), 3.16 (q, J=6.3 Hz, 2H), 1.73-1.50 (m, 4H), 1.43 (s, 9H).

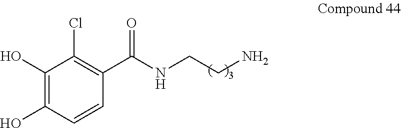

¹H NMR (300 MHz, DMSO-d₆) δ 8.18 (t, J=5.7 Hz, 1H), 7.78 (s, 3H), 6.77 (d, J=8.2 Hz, 1H), 6.72 (d, J=8.2 Hz, 1H), 3.19 (q, J=6.4 Hz, 2H), 2.82 (q, J=6.9 Hz, 2H), 1.69-1.42 (m, 4H).

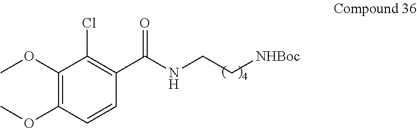

¹H NMR (300 MHz, CDCl₃) δ 7.46 (d, J=8.7 Hz, 1H), 6.87 (d, J=8.7 Hz, 1H), 6.38 (s, 1H), 4.60 (s, 1H), 3.90 (s, 3H), 3.86 (s, 3H), 3.55-3.33 (m, 2H), 3.13 (q, J=6.5 Hz, 2H), 1.65 (p, J=7.1 Hz, 2H), 1.58-1.48 (m, 2H), 1.47-1.35 (m, 11H).

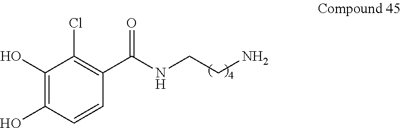

¹H NMR (300 MHz, DMSO-d₆) δ 8.15 (t, J=5.6 Hz, 1H), 7.84 (s, 3H), 6.79 (d, J=8.2 Hz, 1H), 6.71 (d, J=8.2 Hz, 1H), 3.16 (q, J=6.7, 5.1 Hz, 2H), 2.78 (q, J=6.7, 6.2 Hz, 2H), 1.59 (p, J=7.5 Hz, 2H), 1.48 (p, J=6.7 Hz, 3H), 1.36 (p, J=7.8 Hz, 2H).

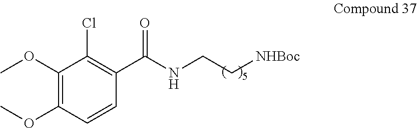

¹H NMR (300 MHz, CDCl₃) δ 7.45 (d, J=8.7 Hz, 1H), 6.86 (d, J=8.7 Hz, 1H), 6.39 (s, 1H), 4.58 (s, 1H), 3.90 (s, 3H), 3.86 (s, 3H), 3.44 (q, J=7.0 Hz, 2H), 3.11 (q, J=6.5 Hz, 2H), 1.63 (p, J=7.1, 6.7 Hz, 3H), 1.55-1.30 (m, 15H).

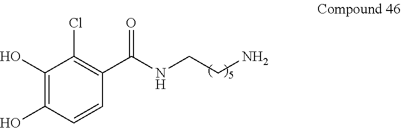

¹H NMR (300 MHz, DMSO-d₆) δ 8.17 (t, J=5.7 Hz, 1H), 7.86 (s, 5H), 6.79 (d, J=8.3 Hz, 1H), 6.70 (d, J=8.2 Hz, 1H), 3.17 (q, J=6.4 Hz, 2H), 2.85-2.73 (m, 2H), 1.63-1.52 (m, 2H), 1.51-1.38 (m, 2H), 1.39-1.21 (m, 4H).

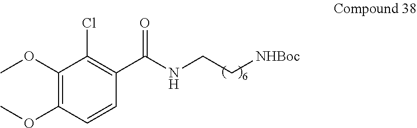

¹H NMR (300 MHz, CDCl₃) δ 7.47 (d, J=8.7 Hz, 1H), 6.87 (d, J=8.6 Hz, 1H), 6.32 (s, 1H), 4.54 (s, 1H), 3.90 (s, 3H), 3.86 (s, 3H), 3.45 (td, J=7.1, 5.8 Hz, 2H), 3.21-3.05 (m, 2H), 1.61 (q, J=7.2 Hz, 2H), 1.53-1.24 (m, 17H).

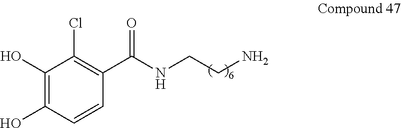

¹H NMR (300 MHz, DMSO-d₆) δ 8.11 (t, J=5.7 Hz, 1H), 7.72 (s, 4H), 6.75 (d, J=8.2 Hz, 1H), 6.68 (d, J=8.2 Hz, 1H), 3.16 (q, J=6.5 Hz, 2H), 2.87-2.72 (m, 2H), 1.61-1.39 (m, 4H), 1.38-1.17 (m, 6H).

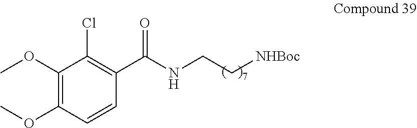

¹H NMR (300 MHz, CDCl₃) δ 7.47 (d, J=8.7 Hz, 1H), 6.87 (d, J=8.7 Hz, 1H), 6.30 (s, 1H), 4.52 (s, 1H), 3.90 (s, 3H), 3.86 (s, 3H), 3.44 (td, J=7.2, 5.8 Hz, 2H), 3.10 (q, J=6.7 Hz, 2H), 1.60 (q, J=7.0 Hz, 2H), 1.53-1.40 (m, 11H), 1.41-1.25 (m, 8H).

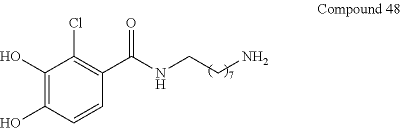

¹H NMR (300 MHz, DMSO-d₆) δ 8.12 (t, J=5.6 Hz, 1H), 7.77 (s, 3H), 6.76 (d, J=8.2 Hz, 1H), 6.69 (d, J=8.2 Hz, 1H), 3.28-3.03 (m, 2H), 2.85-2.71 (m, 2H), 1.68-1.37 (m, 4H), 1.37-1.13 (m, 8H).

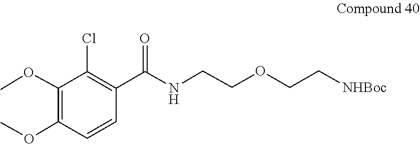 [SMILES: COc1ccc(C(=O)NCCOCCNC(=O)OC(C)(C)C)c(Cl)c1OC]

¹H NMR (300 MHz, CD₃OD) δ 7.21 (d, J=8.6 Hz, 1H), 7.02 (d, J=8.6 Hz, 1H), 3.89 (s, 3H), 3.82 (s, 3H), 3.66-3.57 (m, 2H), 3.56-3.44 (m, 4H), 3.22 (t, J=5.5 Hz, 2H), 1.41 (s, 9H).

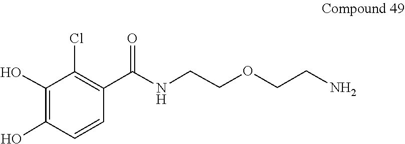 [SMILES: NCCOCCNC(=O)c1ccc(O)c(O)c1Cl]

¹H NMR (300 MHz, DMSO-d₆) δ 8.16 (t, J=5.6 Hz, 1H), 7.86 (s, 4H), 6.85-6.66 (m, 2H), 3.62 (t, J=5.1 Hz, 2H), 3.54 (t, J=5.8 Hz, 2H), 3.39 (q, J=5.8 Hz, 2H), 2.99 (q, J=5.5 Hz, 2H).

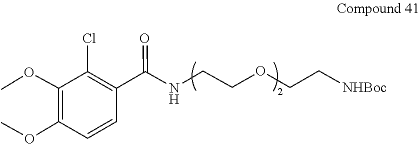

¹H NMR (300 MHz, CD₃OD) δ 7.21 (d, J=8.6 Hz, 1H), 7.03 (d, J=8.6 Hz, 1H), 3.89 (s, 3H), 3.82 (s, 3H), 3.70-3.59 (m, 6H), 3.57-3.43 (m, 4H), 3.20 (t, J=5.9 Hz, 2H), 1.42 (s, 9H).

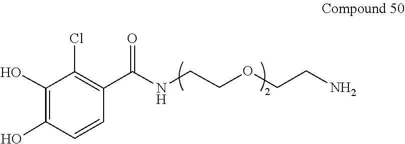

¹H NMR (300 MHz, DMSO-d₆) δ 10.08 (s, 1H), 9.24 (s, 1H), 8.14 (t, J=5.7 Hz, 1H), 7.81 (s, 3H), 6.77-6.66 (m, 2H), 3.65-3.55 (m, 8H), 3.34 (q, J=6.2 Hz, 2H), 2.97 (q, J=5.5 Hz, 2H).

### Synthesis of Compounds 51-54 (Scheme 1).

5,6-Dimethoxyisobenzofuran-1,3-dione (300 mg, 1.44 mmol) and individual N-Boc-diamine (1.1 equivalent) were mixed in 8 mL of toluene in a sealed tube, which was stirred and irradiated in a microwave reactor at 170° C. for 10 min. After cooling down to room temperature, the solvent was removed by reduced pressure followed by the flash chromatography (0→90% EtOAc in hexanes) purification to give corresponding intermediates 51 and 52. Next, the intermediates were deprotected with BBr₃ in DCM according to the above-described procedure to afford compounds 53 and 54 without further purification.

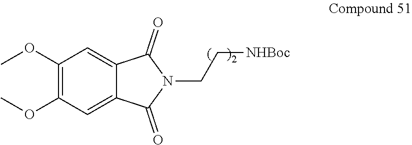

¹H NMR (300 MHz, CDCl₃) δ 7.30 (s, 2H), 5.08 (s, 1H), 3.72 (t, J=6.5 Hz, 2H), 3.13 (q, J=6.4 Hz, 2H), 1.82 (p, J=6.4 Hz, 2H), 1.44 (s, 9H).

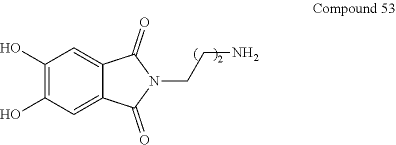

¹H NMR (300 MHz, D₂O) δ 6.86 (s, 2H), 3.77-3.37 (m, 2H), 3.03 (t, J=7.0 Hz, 2H), 2.22-1.47 (m, 2H).

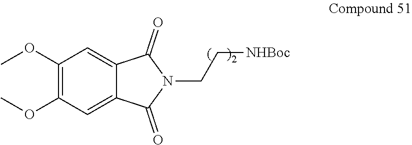

¹H NMR (300 MHz, CDCl₃) δ 7.30 (s, 2H), 4.56 (s, 1H), 3.65 (t, J=7.0 Hz, 2H), 3.16 (q, J=6.6 Hz, 2H), 1.80-1.63 (m, 2H), 1.60-1.46 (m, 2H), 1.43 (s, 9H).

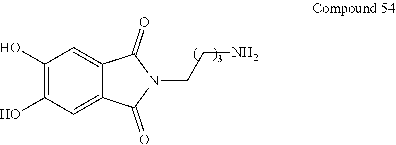

¹H NMR (300 MHz, CD₃OD) δ 7.17 (s, 2H), 3.64 (t, J=6.2 Hz, 2H), 2.99 (t, J=7.0 Hz, 2H), 1.90-1.40 (m, 4H).

### Synthesis of Compounds 55-60 (Scheme 1).

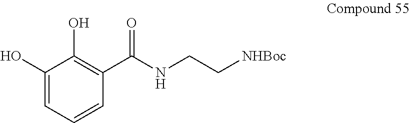 [SMILES: CC(C)(C)OC(=O)NCCNC(=O)c1cccc(O)c1O]

A solution of N-Boc-1,2-diaminoethane (1.27 mL, 8 mmol), 2,3-dihydroxybenzoic acid (616 mg, 4 mmol), and HOBT (648 mg, 4.8 mmol) in THF (10 mL) was attired under N₂ until homogeneous. After 5 min, TEA (1.67 mL, 12 mmol) was added followed by EDC-HCl (2.3 g, 12 mmol). The reaction mixture was stirred for 24 h and the solvent was removed under reduced pressure. The remaining residue was dissolved in 50 mL DCM and washed with 1 N HCl, saturated NaHCO₃ and brine. The DCM solution was concentrated and the residue was purified by flash chromatography (0→5% MeOH in DCM) to obtain 55 (300 mg, 25% yield) as a white solid.

¹H NMR (300 MHz, CDCl₃) δ 12.88 (s, 1H), 7.80 (s, 1H), 7.07-6.92 (m, 2H), 6.75 (dd, J=9.4, 6.6 Hz, 1H), 4.99 (s, 1H), 3.63-3.48 (m, 2H), 3.43 (q, J=5.9 Hz, 2H), 1.44 (s, 9H).

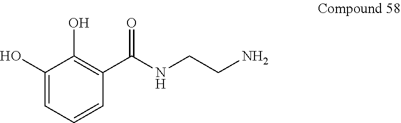 [SMILES: NCCNC(=O)c1cccc(O)c1O]

To compound 55 (300 mg, 1 mmol) was added 4 M HCl/dioxane (10 mL, 40 mmol). After stirring for 40 min at room temperature, the solvent was removed under reduced pressure to obtain compound 58 (190 mg, 97% yield) as an off-white powder solid.

¹H NMR (300 MHz, CD₃OD) δ 7.17 (d, J=8.0 Hz, 1H), 6.86 (d, J=7.8 Hz, 1H), 6.64 (t, J=7.9 Hz, 1H), 3.64-3.57 (m, 2H), 3.08 (t, J=5.9 Hz, 2H).

The other building blocks of this series with different aliphatic chain lengths were synthesized by the same procedure.

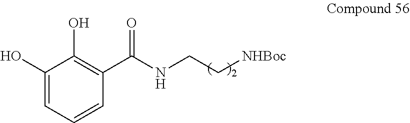

¹H NMR (300 MHz, CDCl₃) δ 7.91 (s, 1H), 7.12 (d, J=8.0 Hz, 1H), 7.04 (dd, J=7.9, 1.4 Hz, 1H), 6.88-6.65 (m, 1H), 4.83 (s, 1H), 3.49 (q, J=6.1 Hz, 2H), 3.26 (q, J=6.3 Hz, 2H), 1.70 (p, J=6.1 Hz, 2H), 1.47 (s, 9H).

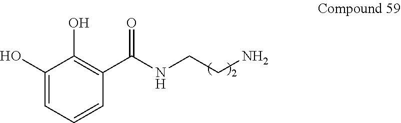

¹H NMR (300 MHz, CD₃OD) δ 7.24 (dd, J=8.1, 1.8 Hz, 1H), 6.94 (dd, J=7.9, 1.8 Hz, 1H), 6.73 (td, J=8.0, 2.0 Hz, 1H), 3.51 (t, J=6.6 Hz, 2H), 3.00 (t, J=7.4 Hz, 2H), 1.97 (p, J=6.8 Hz, 2H).

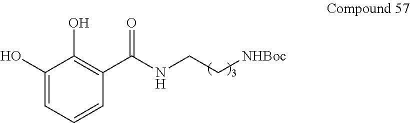

¹H NMR (300 MHz, CDCl₃) δ 7.82 (s, 1H), 7.26 (dd, J=8.1, 1.5 Hz, 1H), 7.02 (dd, J=7.9, 1.5 Hz, 1H), 6.78-6.52 (m, 1H), 4.88 (s, 1H), 3.53-3.36 (m, 2H), 3.14 (q, J=6.5 Hz, 2H), 1.71-1.51 (m, 4H), 1.43 (s, 9H).

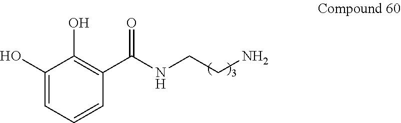

¹H NMR (300 MHz, CD₃OD) δ 7.28 (d, J=8.0 Hz, 1H), 6.96 (d, J=7.8 Hz, 1H), 6.73 (t, J=8.0 Hz, 1H), 3.52-3.36 (m, 2H), 3.24-2.80 (m, 2H), 1.90-1.50 (m, 4H).

### Synthesis of Compounds 61-68 (Scheme 1).

To a solution of 3,4-dimethoxybenzene-1-sulfonyl chloride (710 mg, 3 mmol) in DCM (10 mL) was added N-Boc-diamine (3.3 mmol, 1.1 eq.) and DIPEA (950 μl, 6 mmol) successively, which was stirred at room temperature for 1 h until the completion of the reaction by TLC detection. Then DCM was added to dilute, subsequently followed by the 1N HCl, sat. NAHCO₃ and brine wash respectively. Next, it was dried over Na₂SO₄ which was concentrated and purified by Flash Chromatography, dried in vacuo at last to afford 61-63, 67 with yield >80%. Deprotection procedure was same as above description for compounds 42-50 by BBr₃ in DCM, which gave compounds 64-66 and 68.

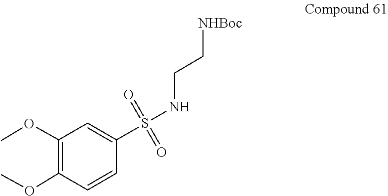 [SMILES: COc1ccc(S(=O)(=O)NCCNC(=O)OC(C)(C)C)cc1OC]

¹H NMR (300 MHz, CDCl₃) δ 7.47 (dd, J=8.5, 2.2 Hz, 1H), 7.33 (d, J=2.2 Hz, 1H), 6.93 (d, J=8.5 Hz, 1H), 5.19 (s, 1H), 4.86 (s, 1H), 3.94 (s, 3H), 3.93 (s, 3H), 3.26-3.17 (m, 2H), 3.10-3.01 (m, 2H), 1.42 (s, 9H).

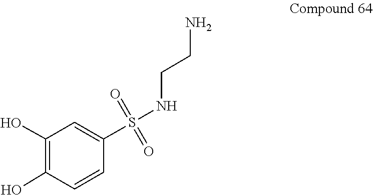 [SMILES: NCCNS(=O)(=O)c1ccc(O)c(O)c1]

¹H NMR (300 MHz, CD₃OD) δ 7.12-6.92 (m, 2H), 6.69 (d, J=8.1 Hz, 1H), 2.94-2.72 (m, 4H).

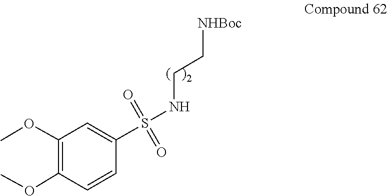

¹H NMR (300 MHz, CDCl₃) δ 7.48 (dd, J=8.4, 2.2 Hz, 1H), 7.35 (d, J=2.1 Hz, 1H), 6.92 (d, J=8.4 Hz, 1H), 5.70-5.44 (m, 1H), 4.71 (t, J=6.5 Hz, 1H), 3.93 (s, 3H), 3.93 (s, 3H), 3.18 (p, J=6.5 Hz, 2H), 2.97 (p, J=6.6 Hz, 2H), 1.63 (h, J=6.4 Hz, 2H), 1.38 (s, 9H).

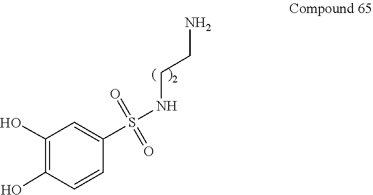

¹H NMR (300 MHz, CD₃OD) δ 7.38-7.12 (m, 2H), 6.91 (d, J=8.2 Hz, 1H), 3.11-2.97 (m, 2H), 2.94 (t, J=6.6 Hz, 2H), 1.85 (p, J=6.8 Hz, 2H).

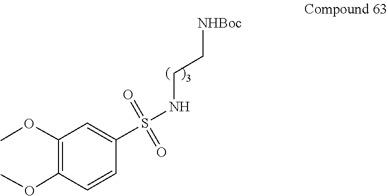

¹H NMR (300 MHz, CDCl₃) δ 7.48 (dd, J=8.4, 2.2 Hz, 1H), 7.34 (d, J=2.2 Hz, 1H), 6.94 (d, J=8.5 Hz, 1H), 4.82 (s, 1H), 4.56 (s, 1H), 3.95 (s, 3H), 3.93 (s, 3H), 3.07 (q, J=6.2 Hz, 2H), 2.94 (q, J=6.1 Hz, 2H), 1.55-1.47 (m, 4H), 1.43 (s, 9H).

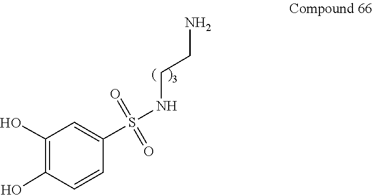

¹H NMR (300 MHz, DMSO-d₆) δ 9.84 (s, 1H), 9.58 (s, 1H), 7.69 (s, 3H), 7.32 (t, J=6.0 Hz, 1H), 7.15 (d, J=2.3 Hz, 1H), 7.08 (dd, J=8.3, 2.3 Hz, 1H), 6.87 (d, J=8.3 Hz, 1H), 2.82-2.70 (m, 2H), 2.70-2.54 (m, 2H), 1.54 (ddd, J=15.1, 9.4, 6.7 Hz, 2H), 1.47-1.29 (m, 2H).

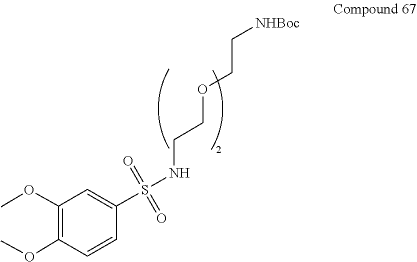

¹H NMR (300 MHz, CDCl₃) δ 7.48 (dt, J=8.5, 2.2 Hz, 1H), 7.35 (d, J=2.3 Hz, 1H), 6.93 (dd, J=8.5, 2.5 Hz, 1H), 5.32 (s, 1H), 5.11 (s, 1H), 3.94 (s, 3H), 3.93 (s, 3H), 3.60-3.47 (m, 8H), 3.33 (q, J=5.4 Hz, 2H), 3.18-3.07 (m, 2H), 1.44 (s, 9H).

### Synthesis of Compounds 69-72 (Scheme 1).

A solution of N-Boc-diamine (1.2 mmol), 5-(benzyloxy)-4-oxo-1,4-dihydropyridine-2-carboxylic acid (245 mg, 1 mmol) and HOBT (648 mg, 4.8 mmol) in THF (10 mL) was stirred under N₂ until homogeneous. After 5 min, TEA (418 μL, 3 mmol) was added followed by EDC-HCl (383 mg, 2 mmol) addition. The reaction mixture was stirred for 24 h and the solvent was removed under reduced pressure. The residue was purified by flash chromatography (0→10% MeOH in DCM) to obtain intermediates 69 (12% yield) 70 (14% yield). The deprotection was performed using BBr₃ (3 equivalents) in DCM, stirring under −78° C. for 30 min, then at room temperature for 30 min. After the reaction completed as judged by LC-MS, the reaction was quenched by MeOH at 0° C. The mixture was concentrated and dried under vacuum overnight to afford siderophore building blocks 71 and 72 as the HBr salts.

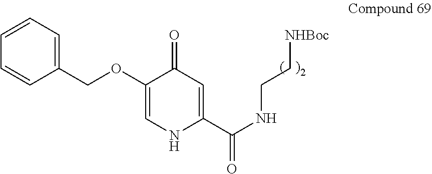

¹H NMR (300 MHz, CD₃OD) δ 7.53-7.40 (m, 3H), 7.43-7.23 (m, 4H), 5.23 (s, 2H), 3.39 (t, J=6.8 Hz, 2H), 3.10 (t, J=6.7 Hz, 2H), 1.73 (p, J=6.9 Hz, 2H), 1.42 (s, 9H).

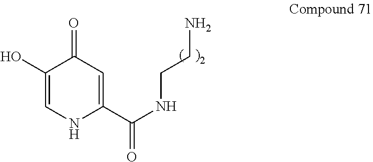

¹H NMR (300 MHz, D₂O) δ 8.12 (s, 1H), 7.63 (s, 1H), 3.53 (t, J=6.8 Hz, 2H), 3.08 (t, J=7.7 Hz, 2H), 2.01 (p, J=7.3 Hz, 2H).

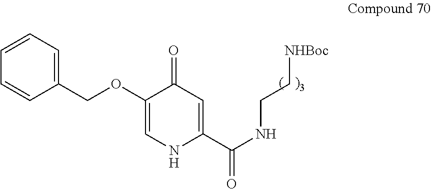

¹H NMR (300 MHz, CDCl₃) δ 8.08 (s, 1H), 7.93 (d, J=6.4 Hz, 1H), 7.81 (s, 1H), 7.51-7.31 (m, 5H), 5.23 (s, 2H), 4.61 (s, 1H), 3.45 (q, J=6.5 Hz, 2H), 3.15 (q, J=6.5 Hz, 2H), 1.64-1.59 (m, 4H), 1.43 (s, 9H).

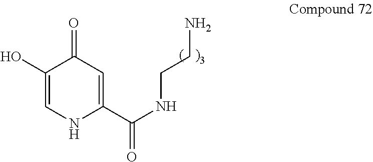

¹H NMR (300 MHz, CD₃OD) δ 8.08 (s, 1H), 7.85 (s, 1H), 3.47 (t, J=6.0 Hz, 2H), 2.98 (t, J=6.1 Hz, 2H), 1.91-1.40 (m, 4H).

### Synthesis of Compounds 73-74 (Scheme 1).

To the solution of siderophore building blocks 58 or 43 (0.4 mmol, 1 equivalent) in methanol (10 mL) was added imidazole-1-sulfonyl azide HCl salt (101 mg, 1.2 equivalents), CuSO₄·5H₂O (10 mg, 0.1 equivalent) and K₂CO₃ (166 mg, 3 equivalents) successively. The mixture was stirred at room temperature for 2 h until the reaction completion was detected by analytic HPLC and LCMS. The resulting turbid grey mixture was quenched with TFA and purified by Prep-RP-HPLC to give the corresponding azido-siderophore building blocks 73 (10% yield) or 74 (55% yield). Note: Intermediate 73 was not pure for characterization and used in the next step without further purification.

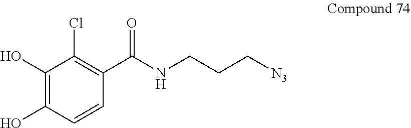 [SMILES: [N-]=[N+]=NCCCNC(=O)c1ccc(O)c(O)c1Cl]

¹H NMR (300 MHz, CDCl₃) δ 7.12 (d, J=8.5 Hz, 1H), 6.84 (dd, J=8.4, 0.9 Hz, 1H), 6.34 (s, 1H), 3.56 (q, J=6.4 Hz, 2H), 3.47 (t, J=6.5 Hz, 2H), 1.92 (p, J=6.6 Hz, 2H).

### Synthesis of Compounds 75-80 (Scheme 1).

To a stirring mixture of 2-chloro-3,4-dimethoxybenzoic acid (3 mmol, 1 equivalent) and amine (3.3 mmol, 1.1 equivalents) in DCM (20 mL) was added DIPEA (6 mmol, 2 equivalents) and HATU (3.9 mmol, 1.3 equivalents). The mixture was stirred at room temperature for 2 h. After the reaction completed, the mixture was concentrated and purified by flash chromatography to give compound 75 and 76 (>80% yield). Next, Dess-Martin periodinane (2.5 equivalents) was added into the DCM (20 mL) solution containing the primary alcohol (1 equivalent), followed by 100 μL H₂O addition subsequently to accelerate the oxidation reaction. After the reaction completed or had no progress, the mixture was filtered to remove the resulting insoluble byproduct followed by the washed with saturated aqueous NaHCO₃ and Na₂S₂O₃ mixture to remove the residual iodine byproduct. The resulting DCM layer was dried over anhydrous Na₂SO₄ and purified by flash chromatography to give compound 77 and 78. At last, the demethylation was performed using BBr₃ (3 equivalents) in DCM, stirring at −78° C. for 30 min, then at room temperature for 30 min. The reaction was quenched with methanol quench, concentrated, and dried under vacuum to give the crude building blocks 79 and 80 which were used in the next step without purification.

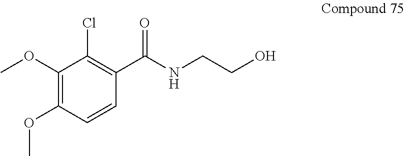 [SMILES: COc1ccc(C(=O)NCCO)c(Cl)c1OC]

¹H NMR (300 MHz, DMSO-d₆) δ 8.22 (t, J=5.5 Hz, 1H), 7.17 (d, J=8.5 Hz, 1H), 7.06 (d, J=8.6 Hz, 1H), 3.85 (s, 3H), 3.74 (s, 3H), 3.48 (t, J=6.3 Hz, 2H), 3.26 (q, J=6.2 Hz, 2H).

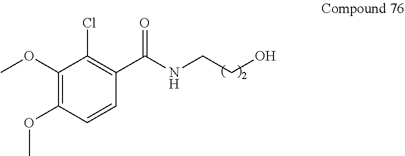

¹H NMR (300 MHz, CDCl₃) δ 7.41 (d, J=8.6 Hz, 1H), 6.87 (d, J=8.7 Hz, 1H), 6.78 (s, 1H), 3.90 (s, 3H), 3.86 (s, 3H), 3.72 (t, J=5.6 Hz, 2H), 3.60 (q, J=6.2 Hz, 2H), 1.85-1.66 (m, 2H).

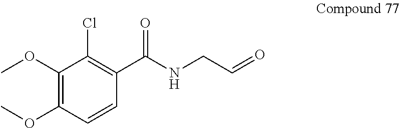 [SMILES: COc1ccc(C(=O)NCC=O)c(Cl)c1OC]

¹H NMR (300 MHz, DMSO-d₆) δ 9.53 (s, 1H), 8.74 (t, J=5.5 Hz, 1H), 7.26 (d, J=8.6 Hz, 1H), 7.11 (d, J=8.6 Hz, 1H), 4.02 (dd, J=5.4, 0.9 Hz, 2H), 3.87 (s, 3H), 3.76 (s, 3H).

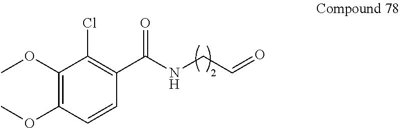

¹H NMR (300 MHz, DMSO-d₆) δ 9.69 (t, J=1.7 Hz, 1H), 8.38 (t, J=5.6 Hz, 1H), 7.13 (d, J=8.5 Hz, 1H), 7.07 (d, J=8.6 Hz, 1H), 3.85 (s, 3H), 3.74 (s, 3H), 3.48 (q, J=6.4 Hz, 2H), 2.65 (td, J=6.6, 1.7 Hz, 2H).

### Synthesis of Target Compounds 1-13, 16-18, 20-21 and Intermediates 81-82 (Scheme 2).

To a mixture of vancomycin hydrochloride (1 equivalent) and siderophore-amine or alkynyl amine (1.5 equivalent) in DMF/DMSO (1:1) was added DIPEA (3 equivalents). The mixture was stirred at room temperature until clear and homogeneous. Then HATU (1 equivalent) dissolved in DMF/DMSO (1:1) was added into the above mixture dropwise. The HATU amount was optimized to avoid the uronium byproduct. The reaction was monitored by analytic RP-HPLC. The reaction was quenched by TFA after about 1-2 hours until no progression was detected by HPLC analysis. The mixture was added dropwise into the cold ether to precipitate, which was subsequently centrifuged. The upper ether layer was removed and the precipitate was dissolved in CH₃CN—H₂O to subject to the RP-HPLC purification. The target peak elution was collected and lyophilized to afford the target compound TFA salt as a white fluffy solid.

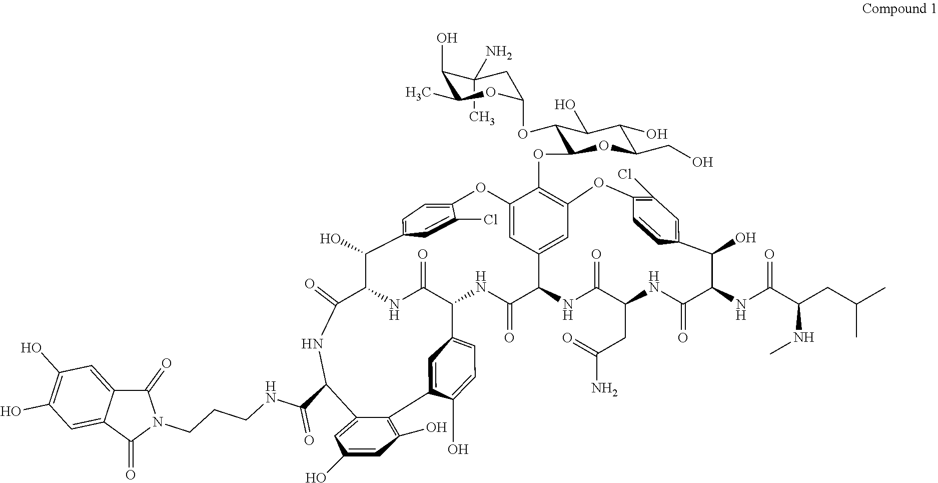

Yield: 49%. HPLC t_{R}=8.719 min.

¹H NMR (300 MHz, DMSO-d₆ with 20 μL D₂O) δ 8.70 (s, 1H), 8.51 (s, 1H), 7.97 (s, 1H), 7.86 (s, 1H), 7.61-7.51 (m, 3H), 7.46 (d, J=8.4 Hz, 1H), 7.33 (d, J=8.4 Hz, 1H), 7.24-7.18 (m, 2H), 7.16 (s, 2H), 6.84-6.62 (m, 3H), 6.37 (d, J=2.3 Hz, 1H), 6.21 (d, J=2.3 Hz, 1H), 5.76 (d, J=7.8 Hz, 1H), 5.60 (s, 1H), 5.33-5.23 (m, 3H), 5.18 (s, 2H), 4.93 (s, 1H), 4.68 (d, J=6.8 Hz, 1H), 4.45 (s, 1H), 4.35 (d, J=5.3 Hz, 1H), 4.29-4.11 (m, 2H), 3.97 (s, 1H), 3.68 (d, J=10.9 Hz, 1H), 3.27 (d, J=5.1 Hz, 2H), 3.21-3.07 (m, 3H), 3.06 (s, 1H), 2.91 (s, 1H), 2.64 (s, 3H), 2.15 (d, J=12.9 Hz, 1H), 1.91 (d, J=12.0 Hz, 1H), 1.80-1.54 (m, 6H), 1.30 (s, 3H), 1.07 (d, J=6.3 Hz, 3H), 0.92 (d, J=5.6 Hz, 3H), 0.87 (d, J=5.6 Hz, 3H);

HRMS (ESI) m/z calcd for C₇₇H₈₅Cl₂N₁₁O₂₇ [M+2H]²⁺ 833.7570, found 833.7566.

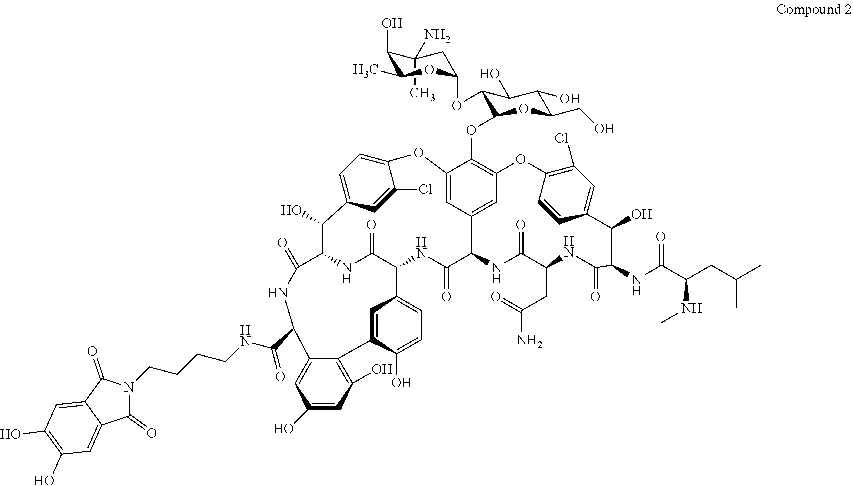

Yield: 45%. HPLC t_{R}=9.041 min.

¹H NMR (300 MHz, DMSO-d₆ with 20 μL D₂O) δ 8.70 (s, 1H), 8.44 (s, 1H), 7.86 (s, 1H), 7.63-7.49 (m, 2H), 7.46 (d, J=8.6 Hz, 1H), 7.40-7.31 (m, 1H), 7.26-7.16 (m, 2H), 7.15 (s, 2H), 6.86-6.63 (m, 3H), 6.52 (d, J=16.5 Hz, 1H), 6.37 (d, J=2.3 Hz, 1H), 6.24 (d, J=2.3 Hz, 1H), 5.76 (d, J=7.7 Hz, 1H), 5.60 (s, 1H), 5.32-5.21 (m, 3H), 5.19 (d, J=4.6 Hz, 2H), 4.93 (s, 1H), 4.68 (d, J=6.8 Hz, 1H), 4.45 (s, 1H), 4.38 (d, J=5.6 Hz, 1H), 4.31-4.10 (m, 2H), 3.97 (s, 1H), 3.68 (d, J=10.9 Hz, 1H), 3.27 (d, J=5.1 Hz, 2H), 3.21-3.10 (m, 3H), 2.64 (s, 3H), 2.16 (d, J=11.2 Hz, 1H), 1.91 (d, J=11.4 Hz, 1H), 1.77-1.51 (m, 6H), 1.51-1.35 (m, 2H), 1.07 (d, J=6.3 Hz, 3H), 0.92 (d, J=5.5 Hz, 3H), 0.87 (d, J=5.5 Hz, 3H);

HRMS (ESI) m/z calcd for C₇₈H₈₇Cl₂N₁₁O₂₇ [M+2H]²⁺ 840.7648, found 840.7642.

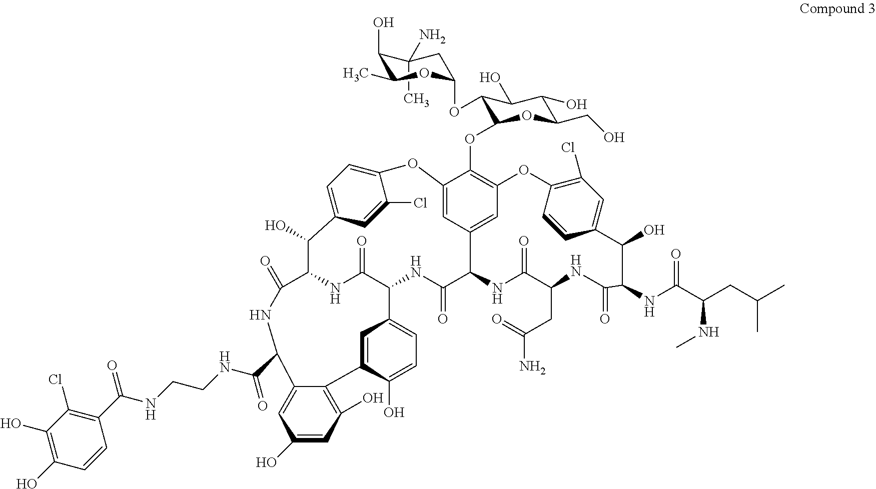

Yield: 75%. HPLC t_{R}=8.249 min.

¹H NMR (300 MHz, DMSO-d₆) δ 8.71 (s, 1H), 8.47 (s, 1H), 8.35 (s, 1H), 8.04 (s, 1H), 7.86 (s, 1H), 7.60-7.50 (m, 2H), 7.46 (d, J=8.5 Hz, 1H), 7.33 (d, J=8.3 Hz, 1H), 7.23-7.14 (m, 2H), 6.85-6.69 (m, 4H), 6.37 (d, J=2.3 Hz, 1H), 6.25 (d, J=2.3 Hz, 1H), 5.76 (d, J=7.8 Hz, 1H), 5.60 (s, 1H), 5.30 (s, 1H), 5.28-5.22 (m, 2H), 5.22-5.15 (m, 2H), 4.97-4.87 (m, 1H), 4.68 (d, J=6.7 Hz, 1H), 4.45 (s, 1H), 4.37 (d, J=5.3 Hz, 1H), 4.23 (s, 2H), 4.03-3.91 (m, 1H), 3.68 (d, J=10.9 Hz, 1H), 3.37-3.21 (m, 5H), 3.18 (s, 1H), 2.94-2.83 (m, 1H), 2.64 (s, 3H), 2.16 (dd, J=16.2, 6.6 Hz, 1H), 1.91 (d, J=11.2 Hz, 1H), 1.81-1.50 (m, 4H), 1.30 (s, 3H), 1.07 (d, J=6.3 Hz, 3H), 0.92 (d, J=5.4 Hz, 3H), 0.87 (d, J=5.6 Hz, 3H);

HRMS (ESI) m/z calcd for C₇₅H₈₄Cl₃N₁₁O₂₆ [M+2H]²⁺ 830.7401, found 830.7401.

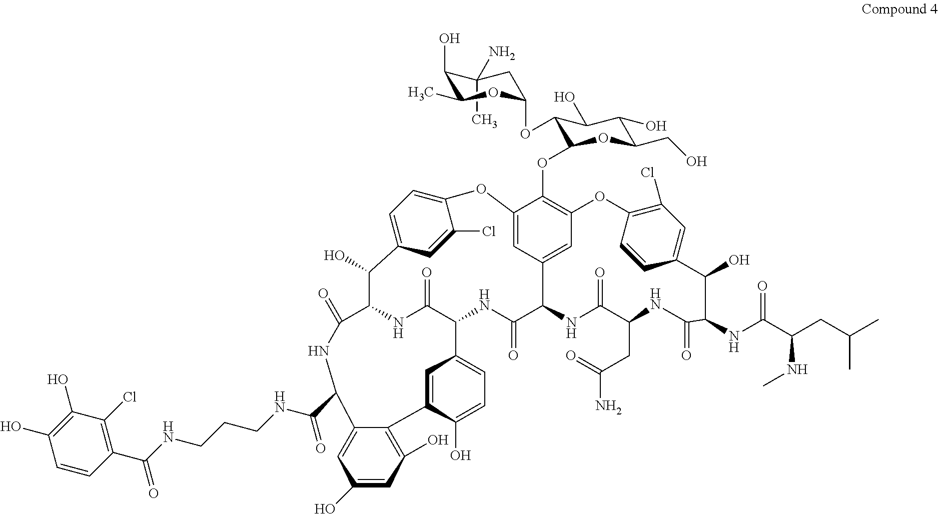

Yield: 85%. HPLC t_{R}=8.200 min.

¹H NMR (300 MHz, DMSO-d₆) δ 8.69 (s, 1H), 8.49 (s, 1H), 7.86 (s, 1H), 7.60-7.49 (m, 2H), 7.45 (d, J=8.1 Hz, 1H), 7.33 (d, J=8.3 Hz, 1H), 7.19 (d, J=8.1 Hz, 2H), 6.83-6.59 (m, 5H), 6.37 (d, J=2.3 Hz, 1H), 6.27 (d, J=2.3 Hz, 1H), 5.76 (d, J=7.8 Hz, 1H), 5.60 (s, 1H), 5.35-5.22 (m, 3H), 5.19 (s, 2H), 4.93 (s, 1H), 4.68 (d, J=6.8 Hz, 1H), 4.45 (s, 1H), 4.37 (d, J=5.3 Hz, 1H), 4.29-4.14 (m, 2H), 4.02-3.85 (m, 1H), 3.68 (d, J=10.8 Hz, 1H), 3.32-3.24 (m, 5H), 3.23-3.12 (m, 2H), 3.08-2.98 (m, 1H), 2.98-2.82 (m, 1H), 2.64 (s, 3H), 2.16 (d, J=9.3 Hz, 1H), 1.90 (d, J=10.8 Hz, 1H), 1.82-1.48 (m, 6H), 1.30 (s, 3H), 1.07 (d, J=6.3 Hz, 3H), 0.91 (d, J=5.4 Hz, 3H), 0.86 (d, J=5.6 Hz, 3H);

HRMS (ESI) m/z calcd for C₇₆H₈₆Cl₃N₁₁O₂₆ [M+2H]²⁺ 837.7479, found 837.7480.

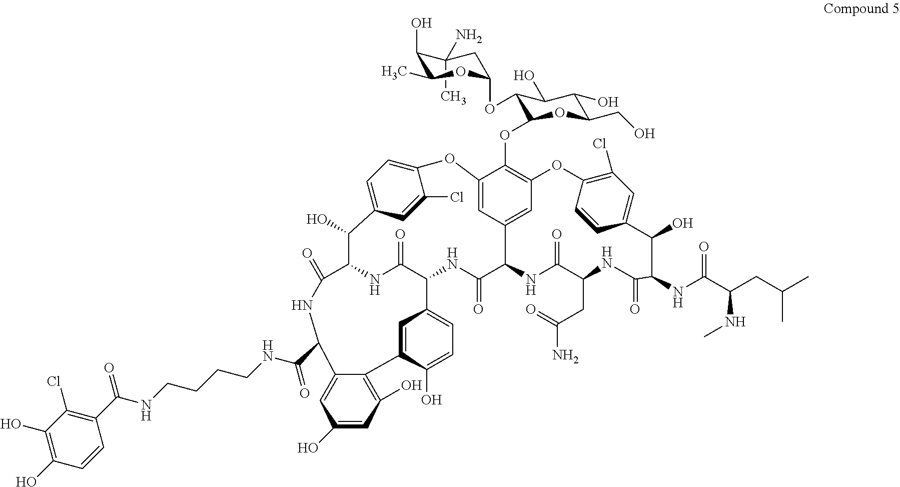

Yield: 50%. HPLC t_{R}=8.396 min.

¹H NMR (300 MHz, DMSO-d₆) δ 8.70 (s, 1H), 8.45 (s, 1H), 7.86 (s, 1H), 7.60-7.50 (m, 2H), 7.47 (d, J=8.7 Hz, 1H), 7.34 (d, J=8.3 Hz, 1H), 7.26-7.12 (m, 2H), 6.83-6.66 (m, 5H), 6.37 (d, J=2.2 Hz, 1H), 6.27 (d, J=2.3 Hz, 1H), 5.76 (d, J=7.8 Hz, 1H), 5.60 (s, 1H), 5.31-5.22 (m, 3H), 5.19 (s, 2H), 5.00-4.85 (m, 1H), 4.68 (d, J=6.7 Hz, 1H), 4.45 (s, 1H), 4.40 (d, J=5.5 Hz, 1H), 4.29-4.10 (m, 2H), 4.04-3.91 (m, 1H), 3.68 (d, J=10.8 Hz, 1H), 3.30-3.24 (m, 2H), 3.25-3.07 (m, 5H), 2.64 (s, 3H), 2.15 (d, J=12.7 Hz, 1H), 1.91 (d, J=12.1 Hz, 1H), 1.83-1.44 (m, 8H), 1.30 (s, 3H), 1.07 (d, J=6.3 Hz, 3H), 0.92 (d, J=5.6 Hz, 3H), 0.87 (d, J=5.7 Hz, 3H);

HRMS (ESI) m/z calcd for C₇₇H₈₈Cl₃N₁₁O₂₆ [M+2H]²⁺ 844.7557, found 844.7558.

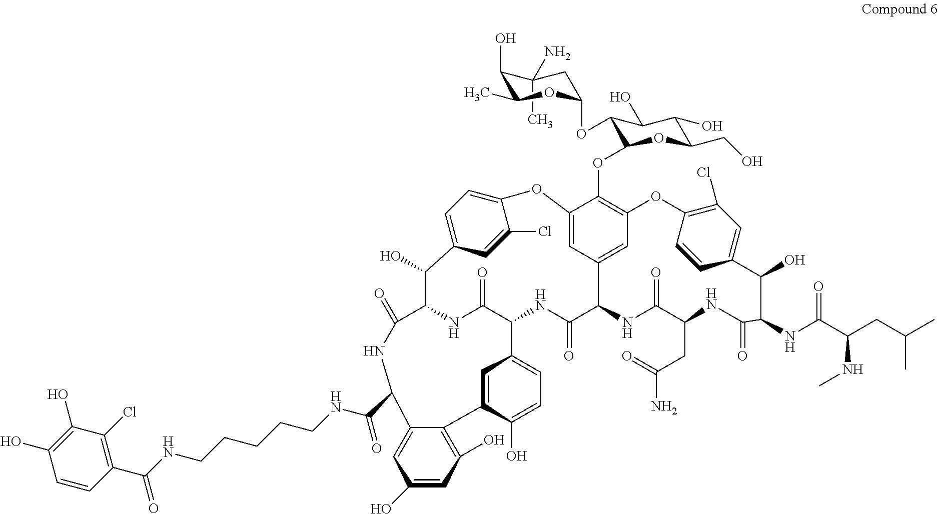

Yield: 79%. HPLC t_{R}=8.489 min.

¹H NMR (300 MHz, DMSO-d₆) δ 8.70 (s, 1H), 8.44 (s, 1H), 7.86 (s, 1H), 7.61-7.50 (m, 2H), 7.47 (d, J=9.5 Hz, 1H), 7.34 (d, J=8.3 Hz, 1H), 7.25-7.10 (m, 2H), 6.82-6.65 (m, 5H), 6.37 (d, J=2.3 Hz, 1H), 6.26 (d, J=2.3 Hz, 1H), 5.76 (d, J=7.7 Hz, 1H), 5.60 (s, 1H), 5.31-5.22 (m, 3H), 5.19 (s, 2H), 4.94 (d, J=3.8 Hz, 1H), 4.68 (d, J=6.7 Hz, 1H), 4.48-4.40 (m, 1H), 4.39 (d, J=5.5 Hz, 1H), 4.30-4.12 (m, 2H), 4.03-3.86 (m, 1H), 3.68 (d, J=10.8 Hz, 1H), 3.32-3.23 (m, 2H), 3.23-3.08 (m, 5H), 2.64 (s, 3H), 2.16 (d, J=10.6 Hz, 1H), 1.91 (d, J=11.6 Hz, 1H), 1.82-1.42 (m, 8H), 1.40-1.21 (m, 5H), 1.07 (d, J=6.3 Hz, 3H), 0.91 (d, J=5.5 Hz, 3H), 0.86 (d, J=5.5 Hz, 3H);

HRMS (ESI) m/z calcd for C₇₈H₉₀Cl₃N₁₁O₂₆ [M+2H]²⁺ 851.7635, found 851.7645.

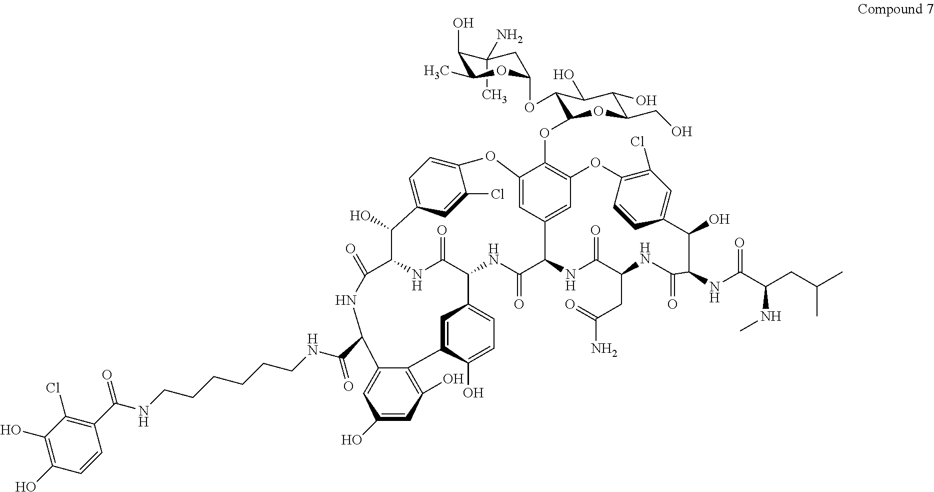

Yield: 80%. HPLC t_{R}=8.906 min.

¹H NMR (300 MHz, DMSO-d₆) δ 8.71 (s, 1H), 8.44 (s, 1H), 7.86 (s, 1H), 7.61-7.50 (m, 2H), 7.47 (d, J=8.6 Hz, 1H), 7.33 (d, J=8.3 Hz, 1H), 7.24-7.15 (m, 2H), 6.82-6.63 (m, 5H), 6.38 (d, J=2.3 Hz, 1H), 6.25 (d, J=2.3 Hz, 1H), 5.75 (d, J=7.7 Hz, 1H), 5.60 (s, 1H), 5.32-5.22 (m, 3H), 5.22-5.15 (m, 3H), 4.93 (s, 1H), 4.68 (d, J=6.8 Hz, 1H), 4.45 (d, J=5.2 Hz, 1H), 4.38 (d, J=5.4 Hz, 1H), 4.28-4.11 (m, 2H), 4.05-3.88 (m, 1H), 3.49-3.38 (m, 1H), 3.31-3.24 (m, 2H), 3.22-3.06 (m, 5H), 2.64 (s, 3H), 2.15 (d, J=10.5 Hz, 1H), 1.91 (d, J=7.6 Hz, 1H), 1.84-1.40 (m, 8H), 1.41-1.22 (m, 7H), 1.07 (d, J=6.2 Hz, 3H), 0.92 (d, J=5.5 Hz, 3H), 0.87 (d, J=5.5 Hz, 3H);

HRMS (ESI) m/z calcd for C₇₉H₉₂Cl₃N₁₁O₂₆ [M+2H]²⁺ 858.7714, found 858.7719.

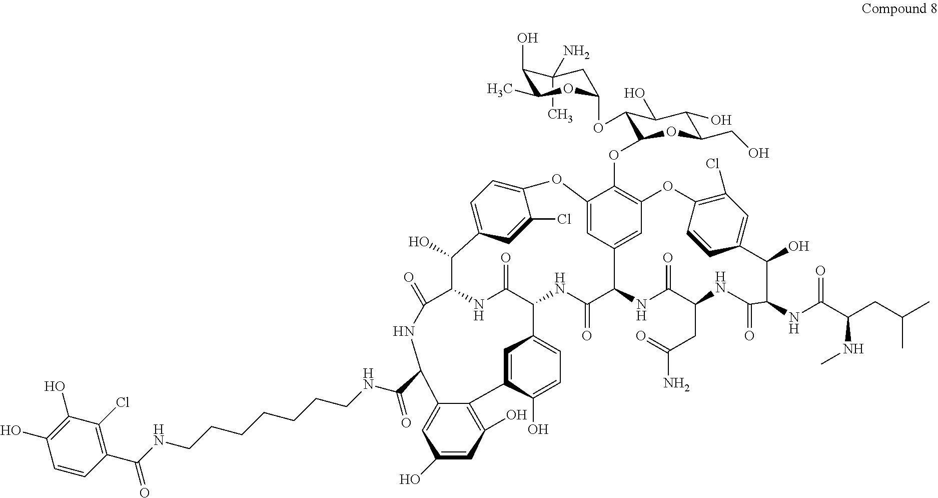

Yield: 80%. HPLC t_{R}=9.070 min.

¹H NMR (300 MHz, DMSO-d₆) δ 8.70 (s, 1H), 8.44 (s, 1H), 7.86 (s, 1H), 7.56 (d, J=8.7 Hz, 1H), 7.52 (s, 1H), 7.47 (d, J=8.7 Hz, 1H), 7.33 (d, J=8.4 Hz, 1H), 7.23-7.14 (m, 2H), 6.82-6.60 (m, 5H), 6.38 (d, J=2.3 Hz, 1H), 6.25 (d, J=2.3 Hz, 1H), 5.76 (d, J=7.7 Hz, 1H), 5.60 (s, 1H), 5.37-5.22 (m, 3H), 5.20 (s, 2H), 4.93 (d, J=4.0 Hz, 1H), 4.68 (d, J=6.7 Hz, 1H), 4.45 (s, 1H), 4.38 (d, J=5.6 Hz, 1H), 4.22 (s, 2H), 4.02-3.89 (m, 1H), 3.68 (d, J=10.9 Hz, 1H), 3.33-3.21 (m, 2H), 3.22-2.99 (m, 5H), 2.64 (s, 3H), 2.12 (d, J=30.0 Hz, OH), 1.91 (d, J=11.3 Hz, 1H), 1.82-1.54 (m, 4H), 1.57-1.39 (m, 4H), 1.38-1.17 (m, 9H), 1.07 (d, J=6.3 Hz, 3H), 0.92 (d, J=5.4 Hz, 3H), 0.87 (d, J=5.3 Hz, 3H);

HRMS (ESI) m/z calcd for C₈₀H₉₄Cl₃N₁₁O₂₆ [M+2H]²⁺ 865.7792, found 865.7786.

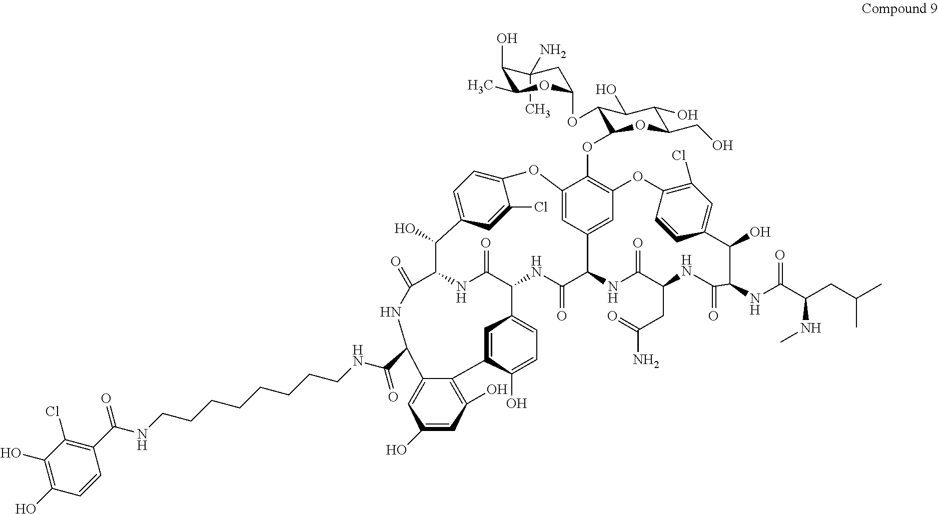

Yield: 79%. HPLC t_{R}=9.516 min.

¹H NMR (300 MHz, DMSO-d₆) δ 8.70 (s, 1H), 8.44 (s, 1H), 7.86 (s, 1H), 7.56 (d, J=8.9 Hz, 1H), 7.52 (s, 1H), 7.47 (d, J=8.7 Hz, 1H), 7.33 (d, J=8.4 Hz, 1H), 7.25-7.14 (m, 2H), 6.81-6.61 (m, 5H), 6.38 (d, J=2.3 Hz, 1H), 6.25 (d, J=2.3 Hz, 1H), 5.76 (d, J=7.5 Hz, 1H), 5.60 (s, 1H), 5.27 (d, J=7.2 Hz, 3H), 5.22-5.11 (m, 2H), 4.93 (d, J=3.7 Hz, 1H), 4.68 (d, J=6.7 Hz, 1H), 4.45 (d, J=5.0 Hz, 1H), 4.38 (d, J=5.5 Hz, 1H), 4.30-4.07 (m, 2H), 4.03-3.88 (m, 1H), 3.68 (d, J=10.6 Hz, 1H), 3.34-3.23 (m, 2H), 3.22-3.05 (m, 5H), 2.64 (s, 3H), 2.15 (d, J=10.1 Hz, 1H), 1.91 (d, J=11.5 Hz, 1H), 1.81-1.55 (m, 4H), 1.54-1.41 (m, 4H), 1.39-1.22 (m, 11H), 1.07 (d, J=6.2 Hz, 3H), 0.92 (d, J=5.4 Hz, 3H), 0.87 (d, J=5.4 Hz, 3H);

HRMS (ESI) m/z calcd for C₈₁H₉₆Cl₃N₁₁O₂₆ [M+2H]²⁺ 872.7870, found 872.7873.

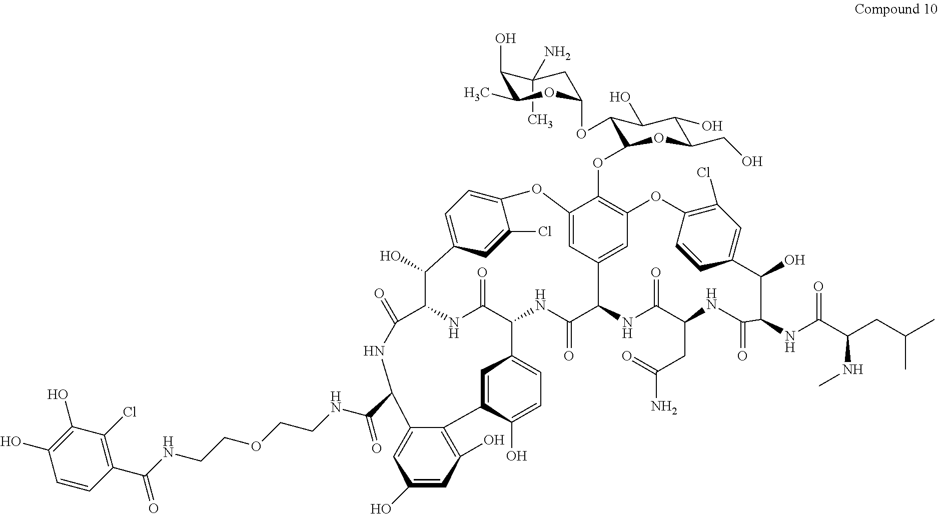

Yield: 41%. HPLC t_{R}=8.175 min.

¹H NMR (300 MHz, DMSO-d₆ with 20 μL CD₃OD and D₂O) δ 8.71 (s, 1H), 8.47 (s, 1H), 7.96 (t, J=4.7 Hz, 1H), 7.87 (s, 1H), 7.56 (d, J=8.8 Hz, 1H), 7.53 (s, 1H), 7.47 (d, J=8.7 Hz, 1H), 7.34 (d, J=8.4 Hz, 1H), 7.26-7.15 (m, 2H), 6.82-6.62 (m, 5H), 6.39 (d, J=2.3 Hz, 1H), 6.27 (d, J=2.3 Hz, 1H), 5.76 (d, J=7.8 Hz, 1H), 5.61 (s, 1H), 5.35-5.22 (m, 3H), 5.20 (s, 2H), 4.94 (d, J=4.0 Hz, 1H), 4.69 (q, J=6.7 Hz, 1H), 4.46 (d, J=5.3 Hz, 1H), 4.41 (d, J=5.5 Hz, 1H), 4.30-4.14 (m, 2H), 4.04-3.91 (m, 1H), 3.41-3.33 (m, 4H), 3.29-3.24 (m, 2H), 3.19 (s, 1H), 2.64 (s, 3H), 2.16 (dd, J=16.1, 7.5 Hz, 1H), 1.92 (d, J=12.3 Hz, 1H), 1.80-1.47 (m, 4H), 1.31 (s, 3H), 1.08 (d, J=6.3 Hz, 3H), 0.92 (d, J=5.5 Hz, 3H), 0.87 (d, J=5.6 Hz, 3H);

HRMS (ESI) m/z calcd for C₇₇H₈₈Cl₃N₁₁O₂₇ [M+2H]²⁺ 852.7532, found 853.7530.

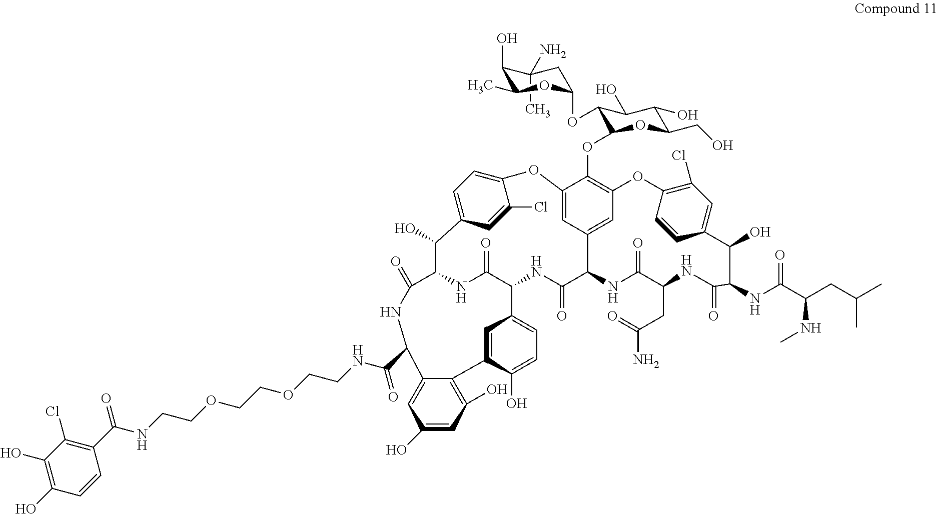

Yield: 70%. HPLC t_{R}=8.218 min.

¹H NMR (300 MHz, DMSO-d₆ with 20 μL D₂O) δ 8.64 (s, 1H), 8.39 (s, 1H), 7.84-7.73 (m, 1H), 7.49 (d, J=8.9 Hz, 1H), 7.45 (s, 1H), 7.40 (d, J=8.7 Hz, 1H), 7.27 (d, J=8.4 Hz, 1H), 7.19-7.02 (m, 2H), 6.83-6.56 (m, 5H), 6.32 (d, J=2.2 Hz, 1H), 6.19 (d, J=2.3 Hz, 1H), 5.69 (d, J=7.7 Hz, 1H), 5.54 (s, 1H), 5.25-5.16 (m, 3H), 5.13 (s, 2H), 4.87 (s, 1H), 4.62 (d, J=6.8 Hz, 1H), 4.50-4.37 (m, 1H), 4.33 (d, J=5.5 Hz, 1H), 4.25-4.04 (m, 2H), 3.98-3.82 (m, 1H), 3.28 (t, J=6.2 Hz, 2H), 3.24-3.15 (m, 2H), 3.12 (s, 1H), 2.58 (s, 3H), 2.09 (d, J=8.9 Hz, 1H), 1.85 (d, J=12.3 Hz, 1H), 1.73-1.41 (m, 4H), 1.24 (s, 3H), 1.01 (d, J=6.3 Hz, 3H), 0.85 (d, J=5.6 Hz, 3H), 0.80 (d, J=5.5 Hz, 3H);

HRMS (ESI) m/z calcd for C₇₉H₉₂Cl₃N₁₁O₂₈ [M+2H]²⁺ 874.7663, found 874.7659.

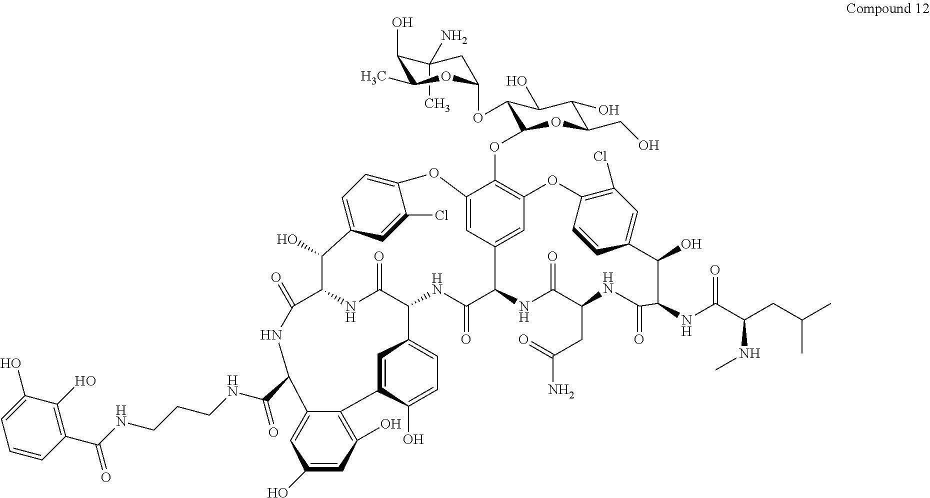

Yield: 68%. HPLC t_{R}=8.701 min.

¹H NMR (300 MHz, DMSO-d₆ with 20 μL D₂O) δ 8.71 (s, 1H), 8.52 (s, 1H), 8.08 (s, 1H), 7.89-7.75 (m, 1H), 7.60-7.51 (m, 2H), 7.46 (d, J=9.4 Hz, 1H), 7.34 (d, J=8.3 Hz, 1H), 7.27 (dd, J=8.1, 1.5 Hz, 1H), 7.23-7.10 (m, 2H), 6.94 (dd, J=7.8, 1.5 Hz, 1H), 6.85-6.62 (m, 4H), 6.38 (d, J=2.3 Hz, 1H), 6.25 (d, J=2.3 Hz, 1H), 5.76 (d, J=7.8 Hz, 1H), 5.60 (s, 1H), 5.33-5.21 (m, 3H), 5.21-5.10 (m, 2H), 4.93 (s, 1H), 4.68 (d, J=6.8 Hz, 1H), 4.46 (d, J=5.1 Hz, 1H), 4.37 (d, J=5.3 Hz, 1H), 4.22 (s, 1H), 4.03-3.88 (m, 1H), 3.68 (d, J=10.8 Hz, 1H), 3.35-3.21 (m, 4H), 3.20-3.10 (m, 2H), 2.64 (s, 3H), 2.23-2.06 (m, 1H), 1.91 (d, J=11.4 Hz, 1H), 1.80-1.46 (m, 6H), 1.30 (s, 3H), 1.07 (d, J=6.2 Hz, 3H), 0.92 (d, J=5.4 Hz, 3H), 0.87 (d, J=5.5 Hz, 3H);

HRMS (ESI) m/z calcd for C₇₆H₈₇Cl₂N₁₁O₂₆ [M+2H]²⁺ 820.7674, found 820.7674.

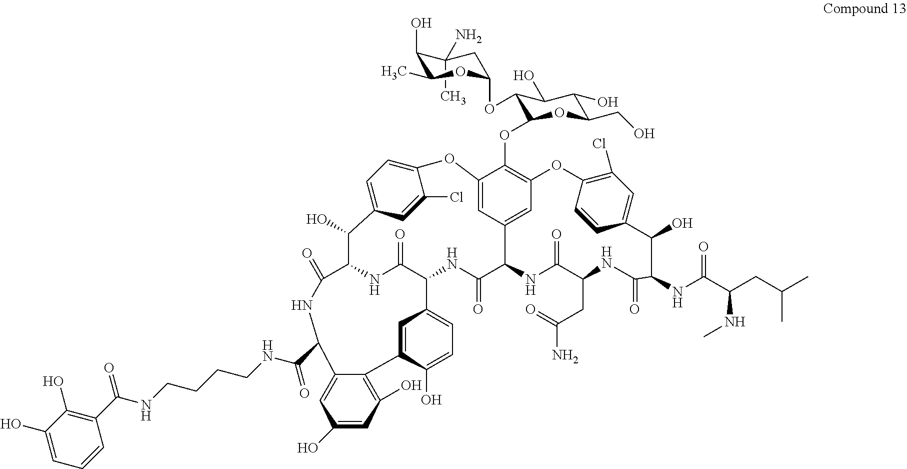

Yield: 69%. HPLC t_{R}=9.015 min.

¹H NMR (300 MHz, DMSO-d₆ with 20 μL D₂O) δ 8.71 (s, 1H), 8.45 (s, 1H), 7.89-7.83 (m, 1H), 7.62-7.50 (m, 2H), 7.47 (d, J=8.6 Hz, 1H), 7.34 (d, J=8.3 Hz, 1H), 7.30-7.24 (m, 1H), 7.24-7.13 (m, 2H), 6.92 (dd, J=7.8, 1.5 Hz, 1H), 6.82-6.62 (m, 4H), 6.38 (d, J=2.3 Hz, 1H), 6.26 (d, J=2.3 Hz, 1H), 5.76 (d, J=7.7 Hz, 1H), 5.60 (s, 1H), 5.34-5.21 (m, 3H), 5.23-5.11 (m, 2H), 4.99-4.81 (m, 1H), 4.68 (d, J=6.8 Hz, 1H), 4.45 (d, J=5.0 Hz, 1H), 4.39 (d, J=5.5 Hz, 1H), 4.30-4.11 (m, 2H), 4.02-3.90 (m, 1H), 3.68 (d, J=10.9 Hz, 1H), 3.36-3.23 (m, 4H), 3.22-3.06 (m, 3H), 2.64 (s, 3H), 2.23-2.08 (m, 1H), 1.91 (d, J=11.6 Hz, 1H), 1.82-1.38 (m, 8H), 1.30 (s, 3H), 1.07 (d, J=6.2 Hz, 3H), 0.92 (d, J=5.4 Hz, 3H), 0.87 (d, J=5.7 Hz, 3H);

HRMS (ESI) m/z calcd for C₇₇H₈₉Cl₂N₁₁O₂₆ [M+2H]²⁺ 827.7752, found 827.7754.

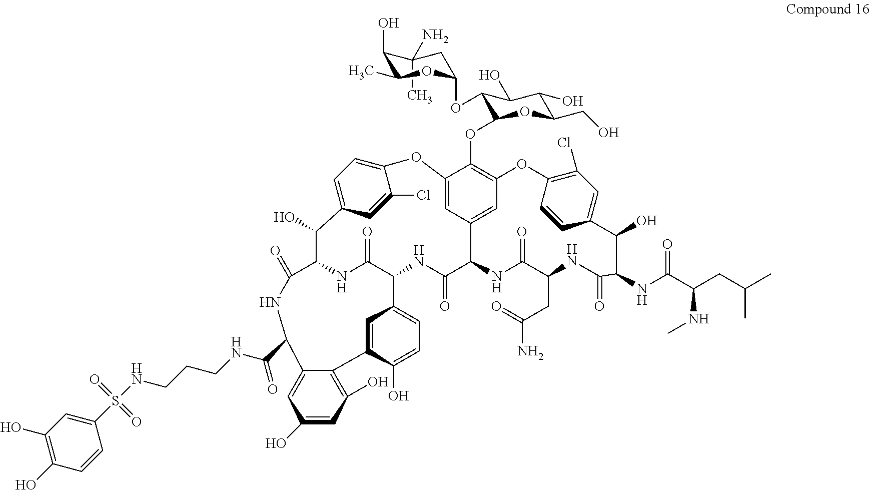

Yield: 69%. HPLC t_{R}=8.388 min.

¹H NMR (300 MHz, DMSO-d₆ with 20 μL D₂O) δ 8.71 (s, 1H), 8.45 (s, 1H), 7.86 (s, 1H), 7.59-7.51 (m, 2H), 7.47 (d, J=8.6 Hz, 1H), 7.34 (d, J=8.4 Hz, 1H), 7.25-7.15 (m, 3H), 7.11 (dd, J=8.3, 2.3 Hz, 1H), 6.89 (d, J=8.3 Hz, 1H), 6.81-6.63 (m, 2H), 6.37 (d, J=2.3 Hz, 1H), 6.21 (d, J=2.3 Hz, 1H), 5.76 (d, J=7.6 Hz, 1H), 5.60 (s, 1H), 5.34-5.21 (m, 3H), 5.22-5.14 (m, 2H), 4.93 (s, 1H), 4.68 (d, J=6.8 Hz, 1H), 4.45 (s, 1H), 4.37 (d, J=5.4 Hz, 1H), 4.21 (s, 2H), 3.96 (d, J=7.1 Hz, 1H), 3.74-3.64 (m, 1H), 3.27 (d, J=4.9 Hz, 2H), 3.18 (s, 1H), 3.16-2.98 (m, 2H), 2.72 (t, J=6.7 Hz, 2H), 2.64 (s, 3H), 2.23-2.06 (m, 1H), 1.91 (d, J=11.8 Hz, 1H), 1.84-1.47 (m, 6H), 1.30 (s, 3H), 1.07 (d, J=6.2 Hz, 3H), 0.92 (d, J=5.5 Hz, 3H), 0.87 (d, J=5.5 Hz, 3H);

HRMS (ESI) m/z calcd for C₇₅H₈₇Cl₂N₁₁O₂₇S [M+2H]2+ calcd for 838.7509, found 838.7510.

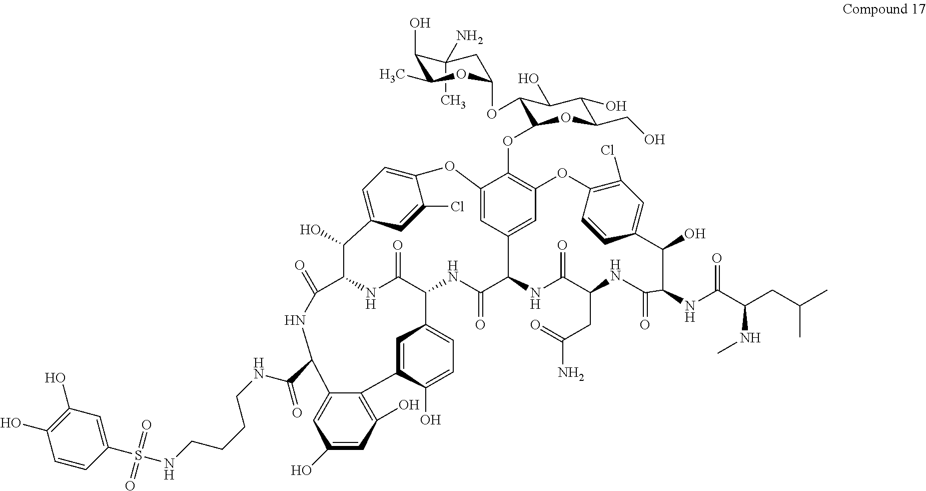

Yield: 59%. HPLC t_{R}=8.707 min.

¹H NMR (300 MHz, DMSO-d₆ with 20 μL D₂O) δ 8.71 (s, 1H), 8.43 (s, 1H), 7.91 (s, 1H), 7.85 (s, 1H), 7.60-7.50 (m, 2H), 7.46 (d, J=8.5 Hz, 1H), 7.33 (d, J=8.3 Hz, 1H), 7.23-7.17 (m, 2H), 7.16 (d, J=2.2 Hz, 1H), 7.10 (dd, J=8.3, 2.3 Hz, 1H), 6.88 (d, J=8.3 Hz, 1H), 6.81-6.66 (m, 3H), 6.37 (d, J=2.3 Hz, 1H), 6.23 (d, J=2.3 Hz, 1H), 5.76 (d, J=7.8 Hz, 1H), 5.60 (s, 1H), 5.30-5.22 (m, 3H), 5.18 (s, 2H), 4.93 (s, 1H), 4.68 (d, J=6.8 Hz, 1H), 4.44 (s, 1H), 4.37 (d, J=5.4 Hz, 1H), 4.28-4.13 (m, 2H), 4.04-3.91 (m, 1H), 3.73-3.60 (m, 2H), 3.32-3.22 (m, 2H), 3.18 (s, 1H), 3.14-3.00 (m, 2H), 2.74-2.58 (m, 5H), 2.24-2.06 (m, 1H), 1.91 (d, J=10.8 Hz, 1H), 1.82-1.51 (m, 4H), 1.50-1.34 (m, 4H), 1.30 (s, 3H), 1.07 (d, J=6.2 Hz, 3H), 0.92 (d, J=5.5 Hz, 3H), 0.87 (d, J=5.6 Hz, 3H);

HRMS (ESI) m/z calcd for C₇₆H₈₉Cl₂N₁₁O₂₇S [M+2H]2+ calcd for 845.7587, found 845.7591.

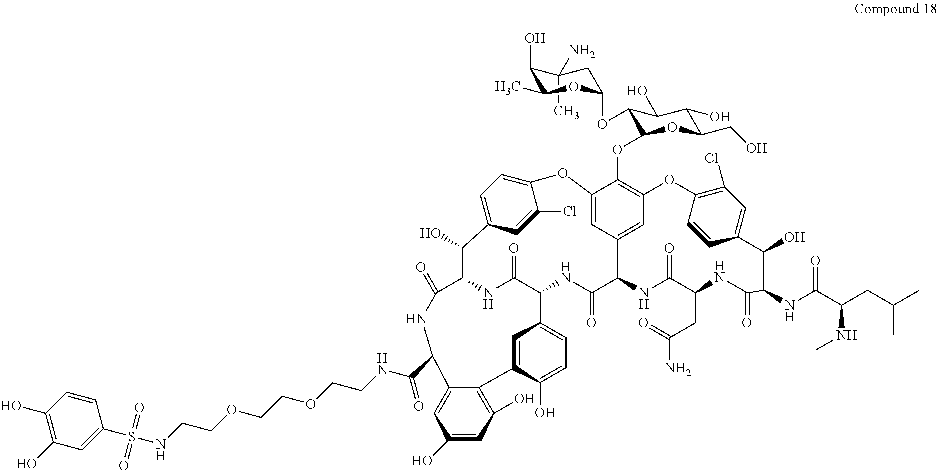

Yield: 19%. HPLC t_{R}=8.628 min.

¹H NMR (300 MHz, DMSO-d₆ with 20 μL D₂O) δ 8.71 (s, 1H), 8.45 (s, 1H), 7.97 (s, 1H), 7.86 (s, 1H), 7.58-7.51 (m, 2H), 7.47 (d, J=8.6 Hz, 1H), 7.34 (d, J=8.4 Hz, 1H), 7.23-7.14 (m, 3H), 7.11 (dd, J=8.3, 2.2 Hz, 1H), 6.88 (d, J=8.3 Hz, 1H), 6.81-6.64 (m, 3H), 6.38 (d, J=2.2 Hz, 1H), 6.25 (d, J=2.3 Hz, 1H), 5.76 (d, J=7.7 Hz, 1H), 5.60 (s, 1H), 5.31-5.22 (m, 3H), 5.18 (s, 2H), 4.93 (s, 1H), 4.68 (d, J=6.7 Hz, 1H), 4.45 (s, 1H), 4.39 (d, J=5.4 Hz, 1H), 4.26-4.15 (m, 2H), 4.02-3.92 (m, 1H), 3.68 (d, J=11.0 Hz, 1H), 3.38 (t, J=6.0 Hz, 2H), 3.34-3.21 (m, 4H), 3.18 (s, 1H), 2.83 (t, J=6.0 Hz, 2H), 2.64 (s, 3H), 2.24-2.09 (m, 1H), 1.91 (d, J=11.9 Hz, 1H), 1.79-1.47 (m, 4H), 1.30 (s, 3H), 1.07 (d, J=6.2 Hz, 3H), 0.92 (d, J=5.4 Hz, 3H), 0.87 (d, J=5.5 Hz, 3H);

HRMS (ESI) m/z calcd for C₇₈H₉₃Cl₂N₁₁O₂₉S [M+2H]²⁺ 875.7692, found 875.7692.

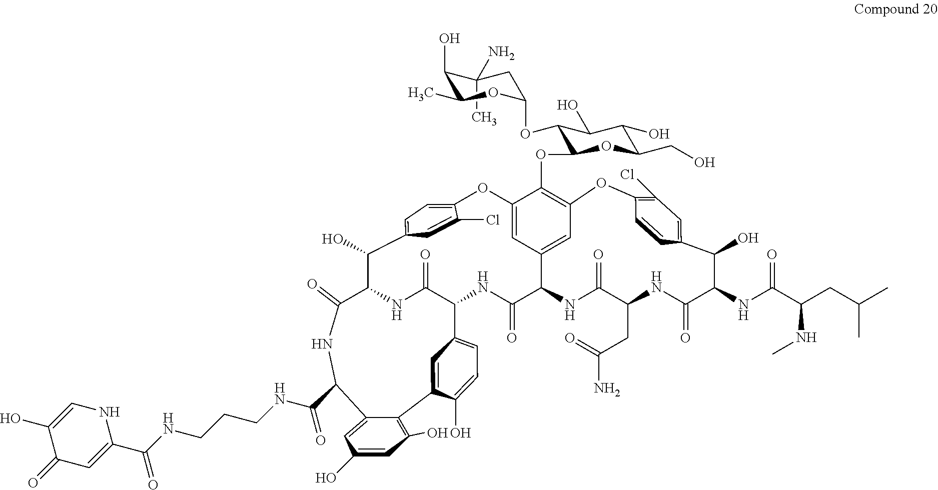

Yield: 49%. HPLC t_{R}=7.799 min.

¹H NMR (300 MHz, DMSO-d₆) δ 8.72 (s, 1H), 8.49 (s, 1H), 7.96 (s, 1H), 7.86 (d, J=5.3 Hz, 1H), 7.59-7.51 (m, 2H), 7.49 (s, 1H), 7.46 (s, 1H), 7.33 (d, J=8.4 Hz, 1H), 7.26-7.11 (m, 2H), 6.85-6.61 (m, 3H), 6.38 (d, J=2.3 Hz, 1H), 6.27 (d, J=2.6 Hz, 1H), 5.76 (d, J=7.7 Hz, 1H), 5.60 (s, 1H), 5.34-5.22 (m, 3H), 5.22-5.12 (m, 2H), 4.93 (s, 1H), 4.68 (d, J=6.8 Hz, 1H), 4.45 (t, J=5.2 Hz, 1H), 4.37 (d, J=5.4 Hz, 1H), 4.31-4.15 (m, 2H), 3.97 (t, J=6.5 Hz, 1H), 3.33-3.22 (m, 4H), 3.18 (s, 1H), 3.03 (s, 1H), 2.64 (s, 3H), 2.22-2.08 (m, 1H), 1.91 (d, J=11.9 Hz, 1H), 1.80-1.47 (m, 6H), 1.30 (s, 3H), 1.07 (d, J=6.3 Hz, 3H), 0.92 (d, J=5.6 Hz, 3H), 0.87 (d, J=5.6 Hz, 3H);

HRMS (ESI) m/z calcd for C₇₅H₈₆Cl₂N₁₂O₂₆ [M+2H]²⁺ 821.2650, found 821.2652.

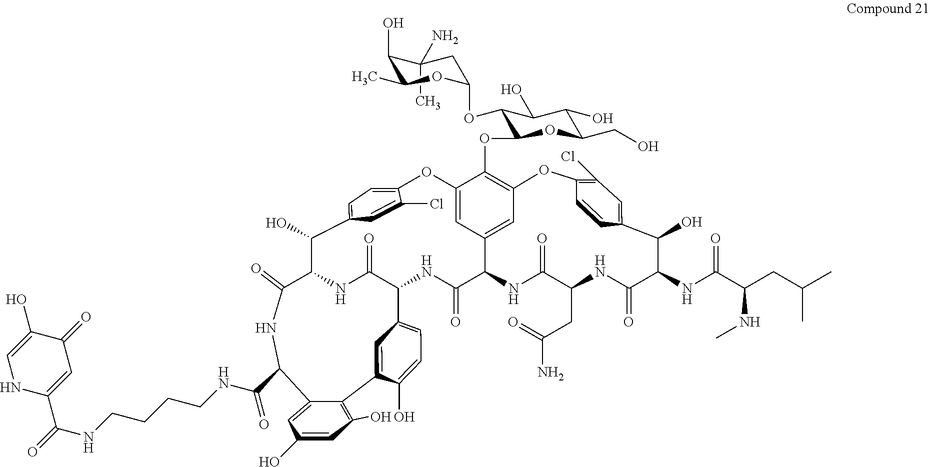

Yield: 42%. HPLC t_{R}=7.808 min.

¹H NMR (300 MHz, DMSO-d₆) δ 8.71 (d, J=5.0 Hz, 1H), 8.44 (s, 1H), 7.93 (s, 1H), 7.86 (s, 1H), 7.61-7.49 (m, 2H), 7.50-7.43 (m, 2H), 7.34 (dd, J=8.4, 4.4 Hz, 1H), 7.20 (d, J=7.4 Hz, 2H), 6.84-6.63 (m, 3H), 6.37 (d, J=2.3 Hz, 1H), 6.26 (d, J=2.6 Hz, 1H), 5.76 (d, J=7.2 Hz, 1H), 5.60 (s, 1H), 5.29-5.21 (m, 3H), 5.21-5.13 (m, 2H), 4.93 (d, J=4.2 Hz, 1H), 4.68 (q, J=6.7 Hz, 1H), 4.45 (t, J=4.4 Hz, 1H), 4.38 (d, J=5.5 Hz, 1H), 4.30-4.16 (m, 2H), 3.97 (t, J=6.3 Hz, 1H), 3.34-3.22 (m, 4H), 3.22-3.07 (m, 2H), 2.64 (s, 3H), 2.24-2.07 (m, 1H), 1.91 (d, J=12.4 Hz, 1H), 1.81-1.57 (m, 4H), 1.58-1.42 (m, 4H), 1.30 (s, 3H), 1.07 (d, J=6.3 Hz, 3H), 0.92 (d, J=5.5 Hz, 3H), 0.87 (d, J=5.5 Hz, 3H);

HRMS (ESI) m/z calcd for C₇₆H₈₈Cl₂N₁₂O₂₆ [M+2H]²⁺ 828.2728, found 828.2727.

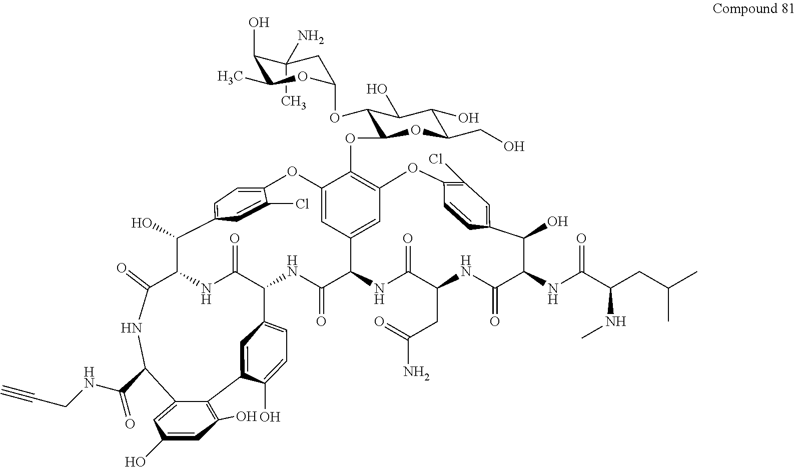

Yield: 67%. HPLC t_{R}=8.290 min.

¹H NMR (300 MHz, DMSO-d₆ with 20 μL D₂O) δ 8.72 (s, 1H), 8.51-8.37 (m, 2H), 7.86 (s, 1H), 7.60-7.51 (m, 2H), 7.47 (d, J=8.5 Hz, 1H), 7.34 (d, J=8.3 Hz, 1H), 7.24-7.15 (m, 2H), 6.39 (d, J=2.3 Hz, 1H), 6.23 (d, J=2.3 Hz, 1H), 5.76 (d, J=7.8 Hz, 1H), 5.60 (s, 1H), 5.30-5.22 (m, 3H), 5.18 (s, 2H), 4.93 (s, 1H), 4.68 (d, J=6.8 Hz, 1H), 4.50-4.38 (m, 1H), 4.21 (d, J=10.7 Hz, 2H), 4.06-3.80 (m, 3H), 3.68 (d, J=11.0 Hz, 1H), 3.31-3.20 (m, 2H), 3.18 (s, 1H), 3.11 (t, J=2.5 Hz, 1H), 2.64 (s, 3H), 2.22-2.05 (m, 1H), 1.91 (d, J=8.2 Hz, 1H), 1.77-1.50 (m, 4H), 1.30 (s, 3H), 1.07 (d, J=6.3 Hz, 3H), 0.92 (d, J=5.5 Hz, 3H), 0.87 (d, J=5.4 Hz, 3H).

MS (ESI) m/z calcd for C₆₉H₇₈Cl₂N₁₀O₂₃ [M+2H]²⁺ 743.2, found 743.4.

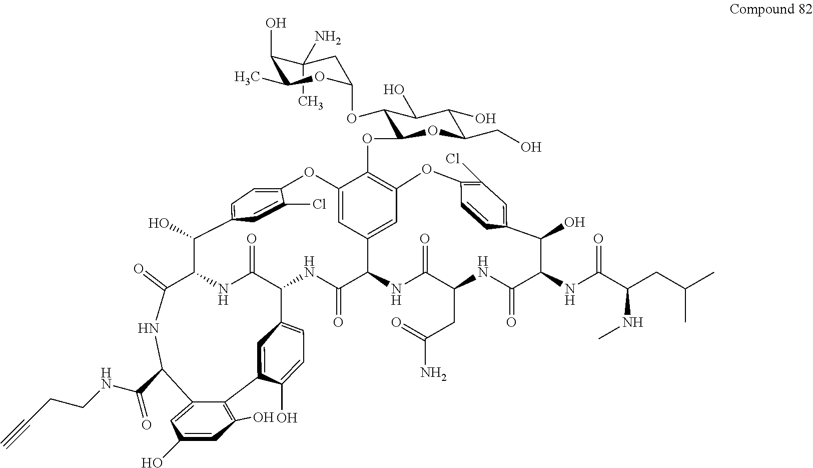

Yield: 60%. HPLC t_{R}=8.350 min.

¹H NMR (300 MHz, DMSO-d₆ with 20 μL D₂O) δ 8.71 (s, 1H), 8.47 (s, 1H), 8.08 (s, 1H), 7.86 (s, 1H), 7.60-7.51 (m, 2H), 7.47 (d, J=8.5 Hz, 1H), 7.34 (d, J=8.3 Hz, 1H), 7.23-7.15 (m, 2H), 6.82-6.63 (m, 3H), 6.37 (d, J=2.3 Hz, 1H), 6.26 (d, J=2.3 Hz, 1H), 5.76 (d, J=7.8 Hz, 1H), 5.60 (s, 1H), 5.31-5.21 (m, 3H), 5.18 (s, 2H), 4.92 (s, 1H), 4.68 (d, J=6.8 Hz, 1H), 4.44 (s, 1H), 4.37 (d, J=5.5 Hz, 1H), 4.29-4.09 (m, 2H), 4.02-3.87 (m, 1H), 3.68 (d, J=10.9 Hz, 1H), 3.37-3.21 (m, 4H), 3.18 (s, 1H), 2.85 (t, J=2.6 Hz, 1H), 2.64 (s, 3H), 2.45-2.30 (m, 2H), 2.25-2.05 (m, 1H), 1.91 (d, J=7.9 Hz, 1H), 1.82-1.46 (m, 4H), 1.30 (s, 3H), 1.07 (d, J=6.2 Hz, 3H), 0.92 (d, J=5.5 Hz, 3H), 0.87 (d, J=5.4 Hz, 3H);

MS (ESI) m/z calcd for C₇₀H₈₀Cl₂N₁₀O₂₃ [M+2H]²⁺ 750.2, found 750.4.

### Synthesis of Target Compounds 22-25 and Intermediates 83-84 (Scheme 3).

Mannich reaction was applied for modifications at the R-position. To a solution of vancomycin hydrochloride (1 equivalent) and amine (siderophore building blocks or alkynyl amine, 10 equivalents) in CH₃OH·H₂O (1:1 or 1:2) was added DIPEA (20 equivalents). The mixture was stirred at room temperature for 5 min, then cooled down to −10° C. After stirring for another 5 min, the 37% formaldehyde solution (1.2 equivalents for intermediates or 1.08 equivalents for final targets) was added into the above mixture, which was allowed to stir for 12-24 h. The reaction was monitored by analytical RP-HPLC and quenched by TFA. The resulting solution was purified directly by Prep-RP-HPLC. The target peak collections were combined and freeze-dried to give target compounds 22-25 and intermediates with alkynyl group 83-84. Note:

Intermediates 83 and 84 were not pure for characterization and used in the click reaction without further purification.

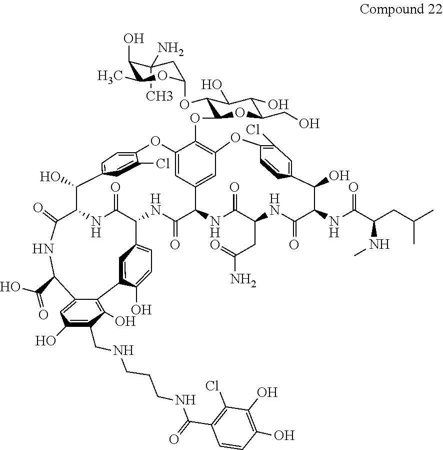

Yield: 33%. HPLC t_{R}=7.850 min.

¹H NMR (300 MHz, DMSO-d₆ with 20 μL D₂O) δ 7.84 (s, 1H), 7.63 (s, 1H), 7.55-7.40 (m, 2H), 7.33 (d, J=8.3 Hz, 1H), 7.22 (d, J=8.4 Hz, 1H), 7.12 (s, 1H), 6.87 (d, J=8.9 Hz, 1H), 6.80 (d, J=8.4 Hz, 1H), 6.77-6.68 (m, 2H), 6.56 (s, 1H), 5.74 (s, 1H), 5.70 (s, 1H), 5.26 (d, J=6.9 Hz, 2H), 5.20-5.07 (m, 3H), 4.83 (s, 1H), 4.67 (d, J=6.7 Hz, 1H), 4.45 (d, J=5.0 Hz, 1H), 4.18-3.90 (m, 4H), 3.68 (d, J=10.9 Hz, 1H), 3.33-3.21 (m, 4H), 3.04-2.95 (m, 2H), 2.60 (s, 3H), 2.11 (d, J=20.8 Hz, 1H), 1.91 (d, J=11.0 Hz, 2H), 1.82-1.44 (m, 5H), 1.07 (d, J=6.3 Hz, 3H);

HRMS (ESI) m/z calcd for C₇₇H₈₈Cl₃N₁₁O₂₇ [M+2H]²⁺ 852.7532, found 852.7525.

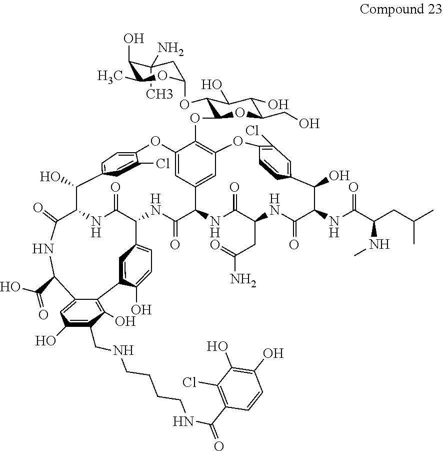

Yield: 50%. HPLC t_{R}=8.132 min.

¹H NMR (300 MHz, DMSO-d₆ with 20 μL D₂O) δ 8.79 (s, 1H), 8.67 (s, 1H), 7.83 (s, 1H), 7.78-7.64 (m, 1H), 7.61 (s, 1H), 7.58-7.42 (m, 2H), 7.33 (dd, J=8.4, 3.6 Hz, 1H), 7.22 (d, J=8.2 Hz, 1H), 7.13 (s, 1H), 6.87 (d, J=8.9 Hz, 1H), 6.80 (d, J=8.4 Hz, 1H), 6.78-6.69 (m, 2H), 6.56 (s, 1H), 5.74 (d, J=6.0 Hz, 1H), 5.69 (s, 1H), 5.27 (d, J=7.7 Hz, 2H), 5.17 (d, J=11.0 Hz, 2H), 5.14-5.07 (m, 1H), 4.84 (s, 1H), 4.68 (d, J=6.7 Hz, 1H), 4.45 (d, J=5.1 Hz, 1H), 4.19-4.07 (m, 2H), 4.07-3.94 (m, 1H), 3.70 (d, 1H), 3.27 (d, J=5.1 Hz, 2H), 3.23-3.18 (m, 2H), 3.00-2.89 (m, 1H), 2.85-2.72 (m, 1H), 2.60 (s, 3H), 2.10 (d, J=21.2 Hz, 1H), 1.91 (d, J=11.8 Hz, 1H), 1.82-1.61 (m, 4H), 1.61-1.41 (m, 4H), 1.32 (s, 3H), 1.07 (d, J=6.2 Hz, 3H), 0.93 (d, J=5.8 Hz, 3H), 0.87 (d, J=6.0 Hz, 3H);

HRMS (ESI) m/z calcd for C₇₈H₉₀Cl₃N₁₁O₂₇ [M+2H]²⁺ 859.7610, found 859.7611.

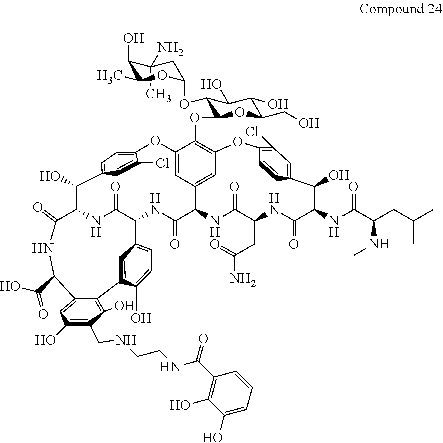

Yield: 20%. HPLC t_{R}=8.230 min.

¹H NMR (300 MHz, DMSO-d₆ with 20 μL D₂O) δ 8.89 (s, OH), 8.80 (s, 1H), 8.67 (s, 1H), 7.83 (s, 1H), 7.63 (s, 1H), 7.50 (t, J=10.1 Hz, 2H), 7.33 (d, J=8.4 Hz, 1H), 7.24 (t, J=7.6 Hz, 2H), 7.12 (s, 1H), 6.96 (dd, J=7.8, 1.4 Hz, 1H), 6.87 (d, J=8.6 Hz, 1H), 6.83-6.64 (m, 3H), 6.56 (s, 1H), 5.74 (d, J=7.5 Hz, 1H), 5.69 (s, 1H), 5.30-5.21 (m, 2H), 5.15 (s, 2H), 5.14-5.07 (m, 1H), 4.83 (s, 1H), 4.68 (d, J=6.7 Hz, 1H), 4.44 (d, J=5.9 Hz, 2H), 4.26-3.95 (m, 5H), 3.73-3.61 (m, 3H), 3.31-3.22 (m, 2H), 3.22-3.07 (m, 3H), 2.81-2.64 (m, 1H), 2.60 (s, 3H), 2.24-2.03 (m, 1H), 1.91 (d, J=10.9 Hz, 1H), 1.81-1.45 (m, 4H), 1.31 (s, 3H), 1.07 (d, J=6.3 Hz, 3H), 0.93 (d, J=5.9 Hz, 3H), 0.88 (d, J=5.9 Hz, 3H);

HRMS (ESI) m/z calcd for C₇₆H₈₇Cl₂N₁₁O₂₇ [M+2H]²⁺ 828.7648, found 828.7646.

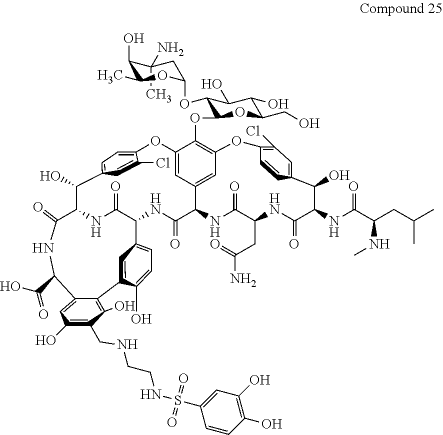

Yield: 14%. HPLC t_{R}=8.172 min.

¹H NMR (300 MHz, DMSO-d₆ with 20 μL D₂O) δ 8.80 (s, 1H), 8.66 (s, 1H), 7.83 (s, 1H), 7.63 (s, 1H), 7.56-7.39 (m, 2H), 7.33 (d, J=8.4 Hz, 1H), 7.24 (s, 1H), 7.21 (s, 1H), 7.17 (d, J=2.3 Hz, 1H), 7.14-7.03 (m, 2H), 6.91 (d, J=8.3 Hz, 1H), 6.86-6.65 (m, 2H), 6.55 (s, 1H), 5.74 (d, J=7.6 Hz, 1H), 5.69 (s, 1H), 5.30-5.21 (m, 2H), 5.20-5.06 (m, 3H), 4.83 (s, 1H), 4.68 (d, J=6.8 Hz, 1H), 4.44 (d, J=5.2 Hz, 1H), 4.18-3.97 (m, 4H), 3.68 (d, J=11.2 Hz, 1H), 3.27 (d, J=5.3 Hz, 2H), 3.18 (s, 1H), 3.01 (s, 3H), 2.60 (s, 3H), 2.22-2.04 (m, 1H), 1.91 (d, J=10.9 Hz, 1H), 1.81-1.44 (m, 4H), 1.31 (s, 3H), 1.07 (d, J=6.3 Hz, 3H), 0.93 (d, J=5.9 Hz, 3H), 0.88 (d, J=5.8 Hz, 3H);

HRMS (ESI) m/z calcd for C₇₅H₈₇Cl₂N₁₁O₂₈S [M+2H]²⁺ 846.7483, found 846.7482.

### Synthesis of Target Compounds 28-29 and Intermediates 85 (Scheme 4).

The reductive amination reaction was applied to the conjugation of siderophore building blocks at V-position. DIPEA (6 equivalents) was added to a mixture of vancomycin HCl salt (75 mg, 0.05 mmol) and siderophore-aldehyde (2 equivalents) in DMF (3 mL). The reaction mixture was stirred at 50° C. for 2 h to form the corresponding imine intermediate which could be detected by analytical HPLC. Afterwards, the mixture was recovered to room temperature followed by the addition of NaCNBH₃ (3 equivalents) solution in methanol (1 mL) and TFA to adjust pH to 3-4 to reduce the imine C—N double bond to C—N single bond. After stirring for 3 h, it was detected no further progression. The mixture was poured into cold-ether (30 mL) dropwise in the 50 mL centrifuge tube. The white precipitate immediately crashed out which was centrifuged to get the precipitate crude. The crude was dissolved in H₂O followed by Prep-RP-HPLC purification. After lyophilizing of the collected target fractions, the final compounds 28, 29 and intermediate 85 were obtained as the white fluffy solid.

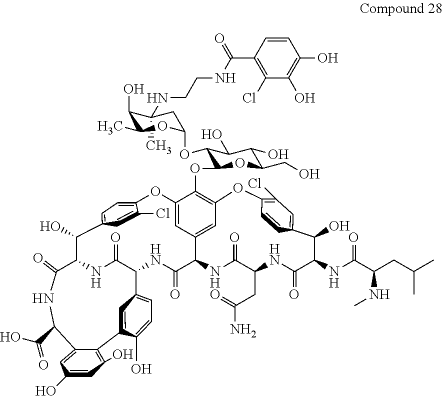

Yield: 40%. HPLC t_{R}=8.206 min.

¹H NMR (300 MHz, DMSO-d₆) δ 8.73 (s, 1H), 8.57 (d, J=5.8 Hz, 1H), 8.34 (t, J=5.6 Hz, 1H), 7.85 (s, 1H), 7.55 (d, J=8.2 Hz, 2H), 7.48 (d, J=8.6 Hz, 1H), 7.34 (d, J=8.3 Hz, 1H), 7.20 (d, J=8.5 Hz, 1H), 7.16 (s, 1H), 6.82 (d, J=8.3 Hz, 1H), 6.79-6.62 (m, 4H), 6.41 (d, J=2.3 Hz, 1H), 6.26 (d, J=2.2 Hz, 1H), 5.76 (d, J=7.7 Hz, 1H), 5.62 (s, 1H), 5.34-5.23 (m, 2H), 5.20-5.14 (m, 2H), 5.12 (s, 1H), 4.92 (d, J=3.8 Hz, 1H), 4.67 (q, J=6.6 Hz, 1H), 4.51-4.35 (m, 2H), 4.19 (d, J=10.7 Hz, 2H), 3.97 (t, J=6.5 Hz, 1H), 3.69 (d, J=10.9 Hz, 1H), 3.34-3.22 (m, 3H), 3.07-2.81 (m, 3H), 2.63 (s, 3H), 2.23-2.08 (m, 1H), 2.00-1.80 (m, 2H), 1.74-1.50 (m, 3H), 1.36 (s, 3H), 1.10 (d, J=6.3 Hz, 3H), 0.92 (d, J=5.6 Hz, 3H), 0.87 (d, J=5.7 Hz, 3H);

HRMS (ESI) m/z calcd for C₇₅H₈₃Cl₃N₁₀O₂₇ [M+2H]²⁺ 831.2321, found 831.2313.

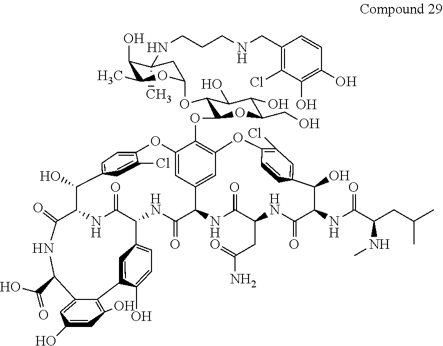

Yield: 29%. HPLC t_{R}=8.086 min.

¹H NMR (300 MHz, DMSO-d₆) δ 8.73 (s, 1H), 8.57 (s, 1H), 8.30 (t, J=5.9 Hz, 1H), 7.85 (s, 1H), 7.62-7.51 (m, 2H), 7.47 (d, J=8.6 Hz, 1H), 7.33 (d, J=8.3 Hz, 1H), 7.20 (d, J=8.6 Hz, 1H), 7.17 (s, 1H), 6.84-6.61 (m, 5H), 6.41 (d, J=2.3 Hz, 1H), 6.26 (d, J=2.3 Hz, 1H), 5.76 (d, J=7.8 Hz, 1H), 5.62 (s, 1H), 5.36-5.25 (m, 2H), 5.22-5.05 (m, 3H), 4.93 (s, 1H), 4.65 (d, J=6.9 Hz, 1H), 4.51-4.38 (m, 2H), 4.26-4.10 (m, 2H), 3.96 (s, 1H), 3.68 (d, J=10.8 Hz, 1H), 3.34-3.18 (m, 4H), 2.95-2.75 (m, 2H), 2.63 (s, 3H), 2.16 (d, J=9.3 Hz, 1H), 2.00 (d, J=10.6 Hz, 1H), 1.89-1.73 (m, 2H), 1.73-1.49 (m, 3H), 1.35 (s, 3H), 1.10 (d, J=6.3 Hz, 3H), 0.92 (d, J=5.5 Hz, 3H), 0.87 (d, J=5.6 Hz, 3H);

HRMS (ESI) m/z calcd for C₇₆H₈₅Cl₃N₁₆O₂₇ [M+2H]²⁺ 838.2399, found 838.2392.

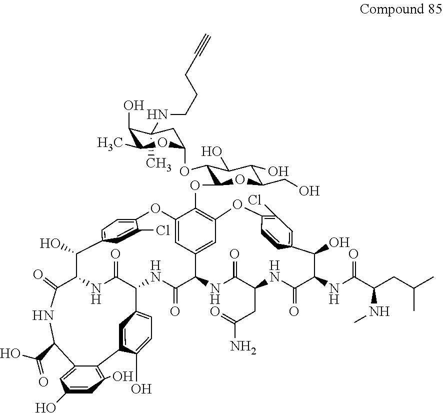

Yield: 12%. HPLC t_{R}=8.367 min.

¹H NMR (300 MHz, DMSO-d₆) δ 8.72 (s, 1H), 8.56 (s, 1H), 7.85 (d, J=1.9 Hz, 1H), 7.59-7.50 (m, 2H), 7.47 (dd, J=8.3, 1.8 Hz, 1H), 7.33 (d, J=8.3 Hz, 1H), 7.20 (d, J=8.5 Hz, 1H), 7.17 (s, 1H), 6.82-6.66 (m, 3H), 6.41 (d, J=2.3 Hz, 1H), 6.26 (d, J=2.3 Hz, 1H), 5.75 (d, J=7.8 Hz, 1H), 5.61 (s, 1H), 5.34-5.24 (m, 2H), 5.19 (d, J=3.6 Hz, 1H), 5.17 (d, J=1.9 Hz, 1H), 5.12 (s, 1H), 4.93 (d, J=4.1 Hz, 1H), 4.64 (q, J=6.6 Hz, 1H), 4.52-4.38 (m, 2H), 4.28-4.11 (m, 2H), 3.96 (t, J=7.2 Hz, 1H), 3.68 (d, J=10.9 Hz, 1H), 3.34-3.21 (m, 3H), 2.95-2.71 (m, 3H), 2.64 (s, 3H), 2.27 (td, J=7.2, 2.7 Hz, 2H), 2.21-2.08 (m, 1H), 1.98 (d, J=10.3 Hz, 1H), 1.87-1.48 (m, 6H), 1.34 (s, 3H), 1.09 (d, J=6.3 Hz, 3H), 0.92 (d, J=5.5 Hz, 3H), 0.87 (d, J=5.6 Hz, 3H);

MS (ESI) m/z calcd for C₇₁H₈₁Cl₂N₉O₂₄ [M+2H]²⁺ 757.7, found 757.6.

### Synthesis of Target Compound 30 and Intermediates 86 (Scheme 5).

The N-terminus modification for vancomycin adopted the selective reductive amination reaction. To a stirring mixture of vancomycin hydrochloride (200 mg, 135 μmol) and 2-chloro-3,4-dihydroxy-N-(2-oxoethyl)benzamide (3 equivalents) in H₂O (3 mL) was added CH₃CN (3 mL) and acetic acid (1.5 mL), stirring for 5 min to make the mixture homogeneous. Then NaCNBH₃ (10 equivalents) was added to the above reaction mixture, allowing to stir for 2 d. Then N₂ stream was used to blow most of the acetic acid and CH₃CN, followed by Prep-RP-HPLC purification to afford compound 30 (5.5 mg, 2% yield) as a white fluffy solid. The intermediate 86 was also prepared in accordance with above procedure.

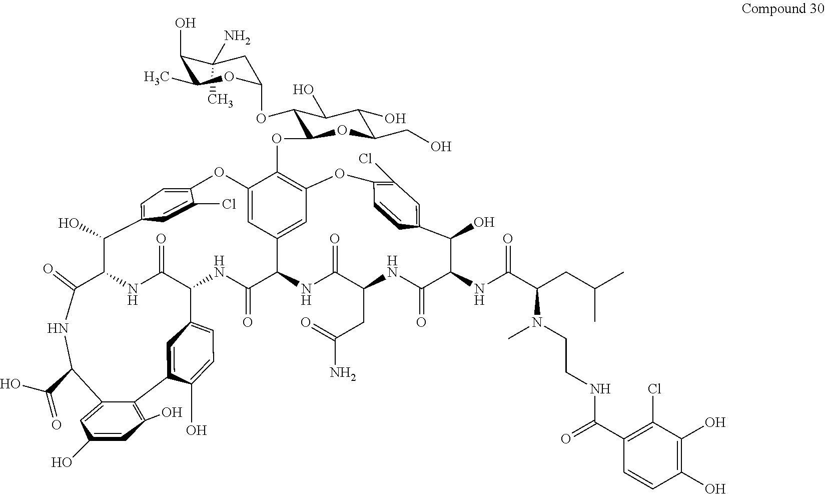

Yield: 2%. HPLC t_{R}=8.695 min.

¹H NMR (300 MHz, DMSO-d₆) δ 8.76 (s, 1H), 8.56 (s, 1H), 7.85 (d, J=1.8 Hz, 1H), 7.54 (d, J=8.9 Hz, 2H), 7.47 (d, J=8.6 Hz, 1H), 7.35 (d, J=8.3 Hz, 1H), 7.20 (d, J=8.3 Hz, 1H), 7.16 (s, 1H), 6.77 (ddd, J=15.3, 11.2, 7.9 Hz, 5H), 6.41 (d, J=2.3 Hz, 1H), 6.26 (d, J=2.4 Hz, 1H), 5.77 (d, J=7.6 Hz, 1H), 5.63 (s, 1H), 5.25 (d, J=7.3 Hz, 2H), 5.22-5.16 (m, 2H), 5.12 (s, 1H), 4.89 (s, 1H), 4.68 (d, J=6.8 Hz, 1H), 4.44 (d, J=5.5 Hz, 1H), 4.26-4.06 (m, 3H), 3.68 (d, J=11.1 Hz, 2H), 3.33-3.21 (m, 3H), 3.18 (s, 1H), 2.94 (s, 2H), 2.90 (s, 1H), 2.74 (s, 1H), 2.64 (s, 1H), 2.28-2.05 (m, 1H), 1.90 (d, J=9.9 Hz, 1H), 1.81-1.67 (m, 1H), 1.54 (d, J=21.7 Hz, 1H), 1.29 (s, 3H), 1.07 (d, J=6.2 Hz, 3H), 0.94 (d, J=6.0 Hz, 3H), 0.88 (d, J=5.7 Hz, 3H);

MS (ESI) m/z calcd for C₇₅H₈₃Cl₃N₁₀O₂₇ [M+2H]²⁺ 831.23, found 831.20.

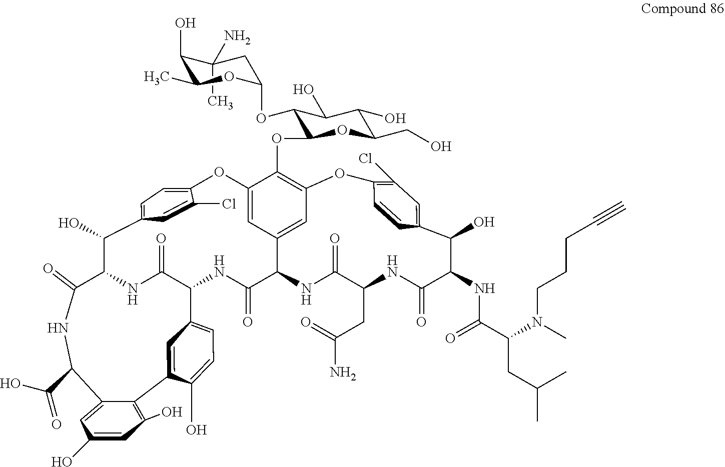

Yield: 2%. HPLC t_{R}=8.951 min.

¹H NMR (300 MHz, DMSO-d₆) δ 8.79 (s, 2H), 8.57 (d, J=5.5 Hz, 1H), 7.85 (s, 1H), 7.61 (s, 1H), 7.53 (d, J=8.5 Hz, 1H), 7.47 (d, J=8.6 Hz, 1H), 7.35 (d, J=8.4 Hz, 1H), 7.20 (d, J=8.4 Hz, 1H), 7.15 (s, 1H), 6.84-6.67 (m, 3H), 6.41 (d, J=2.3 Hz, 1H), 6.26 (d, J=2.3 Hz, 1H), 5.78 (d, J=7.7 Hz, 1H), 5.66 (d, J=10.5 Hz, 1H), 5.25 (d, J=6.7 Hz, 2H), 5.21-5.15 (m, 2H), 5.12 (s, 1H), 4.89 (s, 1H), 4.68 (q, J=7.3, 6.5 Hz, 1H), 4.52-4.35 (m, 2H), 4.27-3.94 (m, 3H), 3.68 (d, J=10.9 Hz, 1H), 3.33-3.21 (m, 3H), 3.18 (s, 2H), 2.94 (s, 1H), 2.87 (s, 3H), 2.37-2.23 (m, 2H), 2.21-2.06 (m, 1H), 1.97-1.65 (m, 4H), 1.60 (d, J=11.7 Hz, 1H), 1.29 (s, 3H), 1.07 (d, J=6.4 Hz, 3H), 0.95 (d, J=6.0 Hz, 3H), 0.88 (d, J=6.1 Hz, 3H);

MS (ESI) m/z calcd for C₇₁H₈₁Cl₂N₉O₂₄ [M+2H]²⁺ 757.75, found 757.60.

### Synthesis of Target Compounds 14, 15, 19, 26, 27, 31 and 32 (Scheme 6).

To a solution of alkynyl vancomycin intermediate (0.025 mmol, 1 equivalent) and siderophore building block with an azido group (0.025 mmol, 1 equivalent) in H₂O/t-BuOH (1:1, 3 mL) was added NaHCO₃ (0.2 mmol, 8 equivalents), CuSO₄·5H₂O (0.05 mmol, 2 equivalents) and sodium ascorbate (0.375 mmol; 15 equivalents) successively. The mixture was stirred at room temperature for 2 h after the reaction completion monitored by analytical RP-HPLC. The reaction was quenched with TFA and the mixture was subsequently purified by Prep-RP-HPLC to give target compounds 14, 15, 19, 26, 27, 31 and 32.

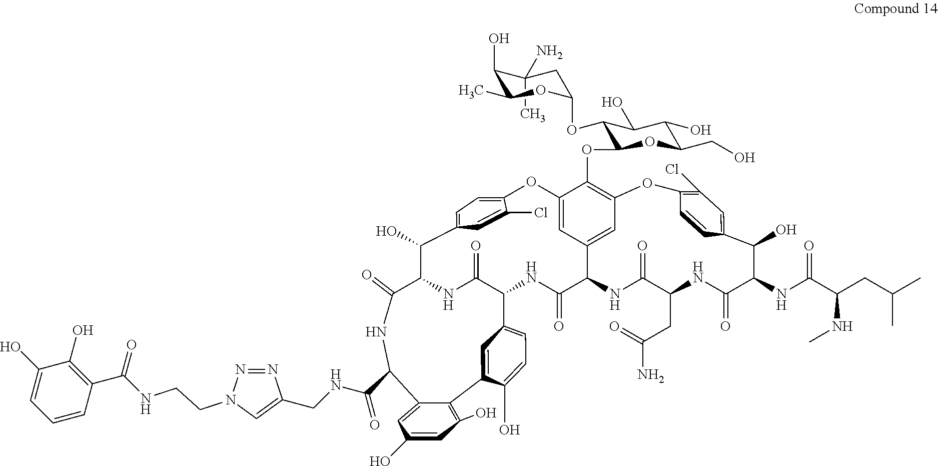

Yield: 75%. HPLC t_{R}=8.701 min.

¹H NMR (300 MHz, DMSO-d₆ with 20 μL D₂O with 20 μL D₂O) δ 8.91 (s, 1H), 8.72 (s, 1H), 8.47 (d, J=10.8 Hz, 2H), 7.87 (d, J=13.0 Hz, 2H), 7.60-7.50 (m, 2H), 7.47 (d, J=8.6 Hz, 1H), 7.34 (d, J=8.3 Hz, 1H), 7.27-7.10 (m, 3H), 6.93 (dd, J=7.8, 1.4 Hz, 1H), 6.84-6.61 (m, 4H), 6.38 (d, J=2.4 Hz, 1H), 6.22 (d, J=2.4 Hz, 1H), 5.76 (d, J=7.9 Hz, 1H), 5.60 (s, 1H), 5.36-5.20 (m, 3H), 5.20-5.10 (m, 2H), 4.93 (s, 1H), 4.68 (d, J=6.8 Hz, 1H), 4.60-4.49 (m, 1H), 4.48-4.35 (m, 3H), 4.31-4.13 (m, 2H), 3.96 (s, 1H), 3.79-3.61 (m, 3H), 3.27 (d, J=5.1 Hz, 2H), 3.18 (s, 1H), 2.64 (s, 3H), 2.26-2.06 (m, 1H), 1.90 (d, J=8.4 Hz, 1H), 1.78-1.49 (m, 4H), 1.30 (s, 3H), 1.07 (d, J=6.2 Hz, 3H), 0.92 (d, J=5.3 Hz, 3H), 0.87 (d, J=5.6 Hz, 4H);

HRMS (ESI) m/z calcd for C₇₈H₈₈Cl₂N₁₄O₂₆ [M+2H]²⁺ 854.2759, found 854.2753.

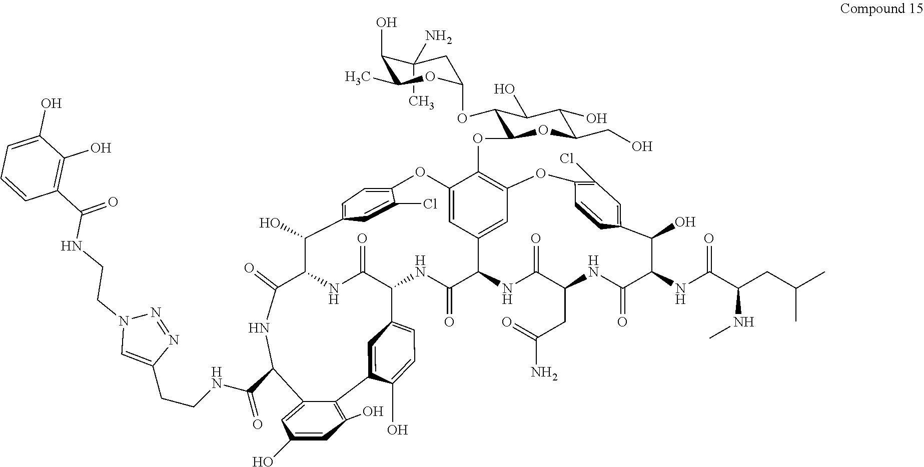

Yield: 39%. HPLC t_{R}=8.735 min.

¹H NMR (300 MHz, DMSO-d₆ with 20 μL D₂O) δ 8.73 (s, 1H), 8.48 (s, 1H), 8.14 (s, 1H), 7.89 (s, 1H), 7.85 (s, 1H), 7.59-7.51 (m, 2H), 7.48 (d, J=8.5 Hz, 1H), 7.34 (d, J=8.3 Hz, 1H), 7.26-7.14 (m, 3H), 6.93 (d, J=7.7 Hz, 1H), 6.78 (d, J=8.6 Hz, 1H), 6.74-6.64 (m, 2H), 6.37 (s, 1H), 6.25 (s, 1H), 5.76 (d, J=8.4 Hz, 1H), 5.61 (s, 1H), 5.27 (d, J=10.1 Hz, 2H), 5.19 (s, 2H), 4.92 (s, 1H), 4.58-4.42 (m, 3H), 4.42-4.34 (m, 1H), 4.29-4.12 (m, 2H), 4.03-3.89 (m, 1H), 3.81-3.63 (m, 4H), 3.32-3.21 (m, 2H), 3.18 (s, 1H), 2.91-2.81 (m, 2H), 2.80-2.71 (m, 2H), 2.63 (s, 3H), 2.25-2.05 (m, 1H), 1.90 (d, J=9.4 Hz, 1H), 1.80-1.47 (m, 4H), 1.07 (d, J=6.2 Hz, 3H), 0.92 (d, J=5.5 Hz, 3H), 0.87 (d, J=5.4 Hz, 3H);

HRMS (ESI) m/z calcd for C₇₉H₉₀Cl₂N₁₄O₂₆ [M+2H]²⁺ 861.2837, found 861.2837.

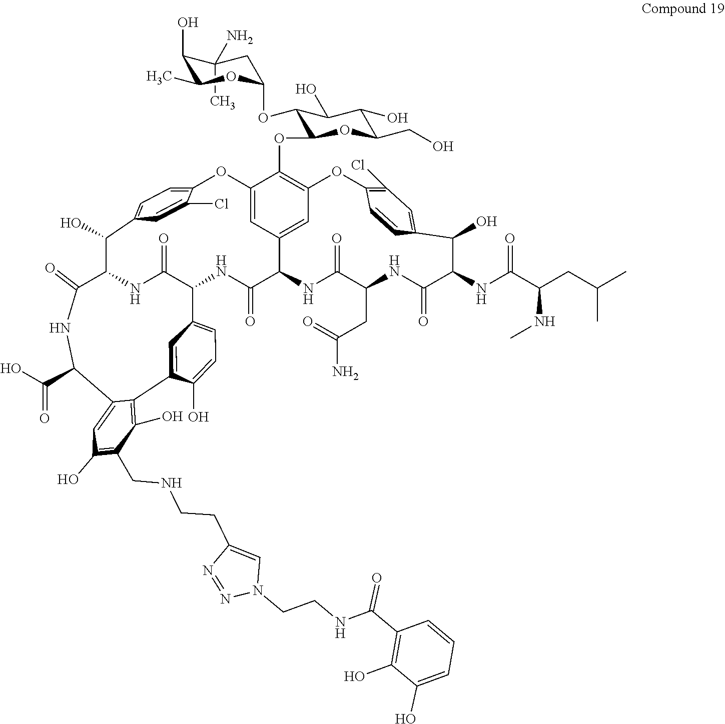

Yield: 30%. HPLC t_{R}=8.354 min.

¹H NMR (300 MHz, DMSO-d₆ with 20 μL D₂O) δ 8.81 (s, 1H), 8.68 (s, 1H), 7.99 (s, 1H), 7.83 (s, 1H), 7.64 (s, 1H), 7.50 (t, J=9.3 Hz, 2H), 7.33 (d, J=8.3 Hz, 1H), 7.23 (d, J=8.5 Hz, 1H), 7.19 (dd, J=8.1, 1.5 Hz, 1H), 7.15-7.07 (m, 1H), 6.93 (dd, J=7.9, 1.5 Hz, 1H), 6.87 (d, J=8.7 Hz, 1H), 6.80 (d, J=8.6 Hz, 2H), 6.70 (t, J=8.0 Hz, 1H), 6.56 (s, 1H), 5.75 (d, J=7.6 Hz, 1H), 5.70 (s, 1H), 5.31-5.21 (m, 2H), 5.19-5.06 (m, 3H), 4.82 (s, 1H), 4.67 (d, J=6.7 Hz, 1H), 4.60-4.50 (m, 2H), 4.49-4.35 (m, 2H), 4.24-3.94 (m, 4H), 3.78-3.68 (m, 2H), 3.66 (s, 1H), 3.31-3.23 (m, 2H), 3.24-3.13 (m, 3H), 3.13-2.95 (m, 3H), 2.76 (d, J=14.0 Hz, 1H), 2.59 (s, 3H), 2.23-2.04 (m, 1H), 1.90 (d, J=8.3 Hz, 1H), 1.79-1.60 (m, 3H), 1.59-1.48 (m, 1H), 1.30 (s, 3H), 1.07 (d, J=6.2 Hz, 3H), 0.93 (d, J=5.8 Hz, 3H), 0.88 (d, J=5.7 Hz, 3H);

HRMS (ESI) m/z calcd for C₈₀H₉₂Cl₂N₁₄O₂₇ [M+2H]²⁺ 876.2890, found 876.2881.

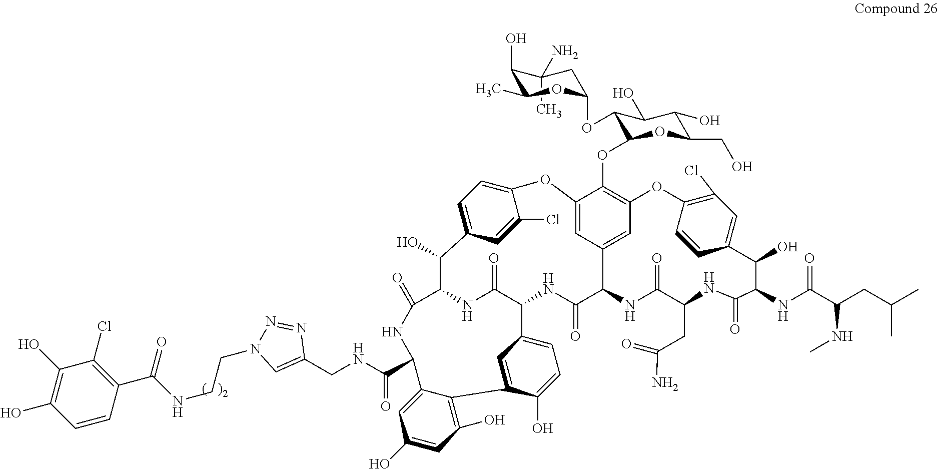

Yield: 39%. HPLC t_{R}=8.213 min.

¹H NMR (300 MHz, DMSO-d₆) δ 8.72 (s, 1H), 8.49 (s, 2H), 8.31 (t, J=5.5 Hz, 1H), 7.89 (s, 1H), 7.85 (s, 1H), 7.55 (d, J=9.0 Hz, 2H), 7.47 (d, J=8.5 Hz, 1H), 7.34 (d, J=8.4 Hz, 1H), 7.20 (d, J=8.6 Hz, 2H), 6.84-6.65 (m, 5H), 6.38 (d, J=2.3 Hz, 1H), 6.22 (d, J=2.3 Hz, 1H), 5.76 (d, J=7.8 Hz, 1H), 5.60 (s, 1H), 5.26 (d, J=6.7 Hz, 3H), 5.19 (s, 2H), 4.93 (s, 1H), 4.68 (d, J=6.8 Hz, 1H), 4.51-4.33 (m, 6H), 4.31-4.11 (m, 2H), 3.96 (s, 1H), 3.68 (d, J=12.5 Hz, 1H), 3.33-3.11 (m, 6H), 2.64 (s, 3H), 2.15 (d, J=10.0 Hz, 1H), 2.05 (q, J=7.2 Hz, 2H), 1.91 (d, J=8.1 Hz, 1H), 1.80-1.46 (m, 4H), 1.30 (s, 3H), 1.07 (d, J=6.2 Hz, 3H), 0.92 (d, J=5.5 Hz, 3H), 0.87 (d, J=5.6 Hz, 3H);

HRMS (ESI) m/z calcd for C₇₉H₈₉Cl₃N₁₄O₂₆ [M+2H]²⁺ 878.2642, found 878.2632.

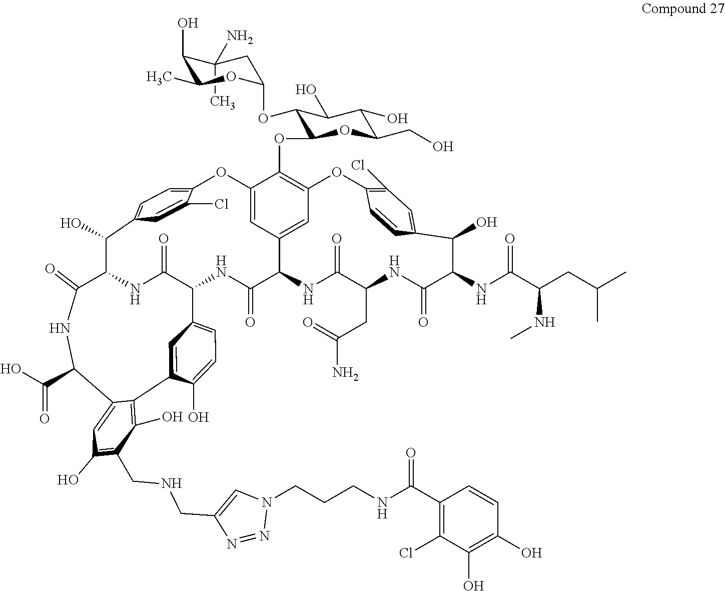

Yield: 18%. HPLC t_{R}=8.080 min.

¹H NMR (300 MHz, DMSO-d₆ with 20 μL D₂O) δ 8.73 (s, 1H), 8.59 (s, 1H), 8.20 (s, 1H), 7.76 (s, 1H), 7.55 (s, 1H), 7.50-7.36 (m, 2H), 7.27 (d, J=8.4 Hz, 1H), 7.15 (d, J=8.4 Hz, 1H), 7.06 (s, 1H), 6.80 (d, J=8.8 Hz, 1H), 6.73 (d, J=8.4 Hz, 1H), 6.70-6.60 (m, 2H), 6.48 (s, 1H), 5.67 (d, J=7.8 Hz, 1H), 5.62 (s, 1H), 5.23-5.13 (m, 2H), 5.06 (d, J=7.5 Hz, 3H), 4.77 (s, 1H), 4.61 (d, J=6.7 Hz, 1H), 4.47-4.27 (m, 4H), 4.18 (s, 2H), 4.14-3.99 (m, 2H), 3.99-3.84 (m, 1H), 3.61 (d, J=11.0 Hz, 1H), 3.25-3.17 (m, 2H), 3.17-3.00 (m, 3H), 2.53 (s, 3H), 2.14-1.89 (m, 1H), 1.84 (d, J=7.7 Hz, 1H), 1.74-1.39 (m, 4H), 1.23 (s, 3H), 1.00 (d, J=6.3 Hz, 3H), 0.86 (d, J=5.9 Hz, 3H), 0.81 (d, J=6.1 Hz, 3H);

HRMS (ESI) m/z calcd for C₈₀H₉₁Cl₃N₁₄O₂₇ [M+2H]²⁺ 893.2695, found 893.2686.

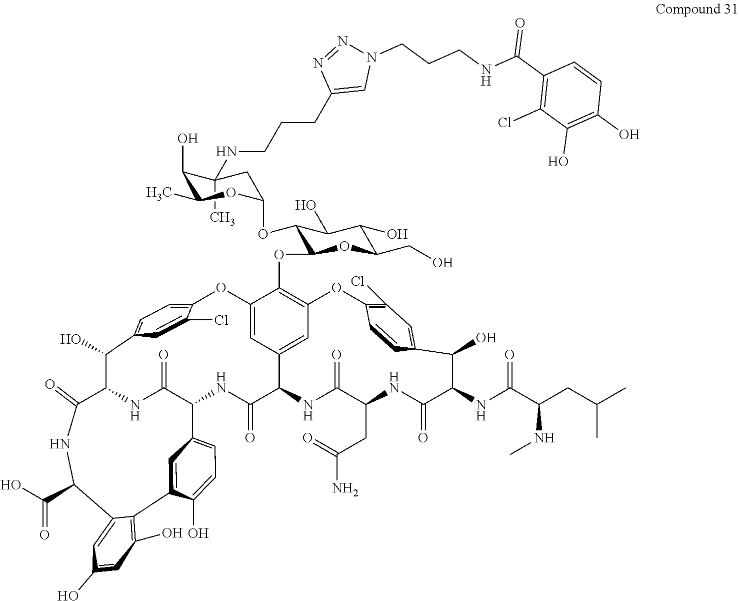

Yield: 71%. HPLC t_{R}=8.467 min.

¹H NMR (300 MHz, DMSO-d₆) δ 8.74 (s, 1H), 8.57 (s, 1H), 8.29 (t, J=5.8 Hz, 1H), 7.90 (s, 1H), 7.85 (s, 1H), 7.59-7.51 (m, 2H), 7.48 (d, J=8.5 Hz, 1H), 7.33 (d, J=8.3 Hz, 1H), 7.20 (d, J=8.7 Hz, 1H), 7.17 (s, 1H), 6.83-6.68 (m, 5H), 6.41 (d, J=2.2 Hz, 1H), 6.26 (d, J=2.3 Hz, 1H), 5.76 (d, J=7.8 Hz, 1H), 5.62 (s, 1H), 5.35-5.25 (m, 2H), 5.19 (d, J=3.5 Hz, 1H), 5.16 (s, 1H), 5.12 (s, 1H), 4.92 (s, 1H), 4.64 (d, J=6.8 Hz, 1H), 4.48-4.42 (m, 2H), 4.42-4.33 (m, 2H), 4.27-4.11 (m, 2H), 3.97 (s, 1H), 3.69 (d, J=10.8 Hz, 1H), 3.32-3.24 (m, 3H), 3.18 (d, J=6.0 Hz, 2H), 3.00-2.78 (m, 2H), 2.76-2.66 (m, 2H), 2.63 (s, 3H), 2.22-2.09 (m, 1H), 2.09-1.76 (m, 6H), 1.75-1.48 (m, 3H), 1.35 (s, 3H), 1.09 (d, J=6.2 Hz, 3H), 0.92 (d, J=5.6 Hz, 3H), 0.87 (d, J=5.6 Hz, 3H);

HRMS (ESI) m/z calcd for C₈₁H₉₂Cl₃N₁₃O₂₇ [M+2H]²⁺ 892.7719, found 892.7720.

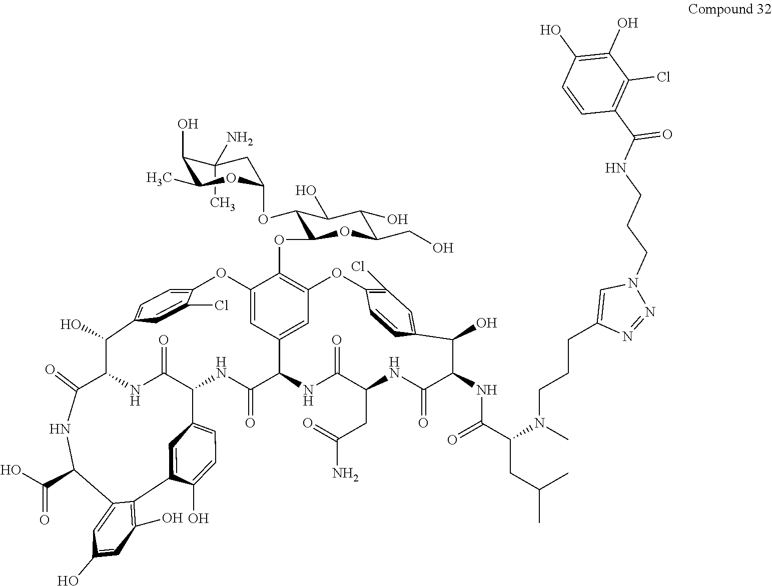

Yield: 85%. HPLC t_{R}=8.873 min.

¹H NMR (300 MHz, DMSO-d₆) δ 8.77 (s, 1H), 8.57 (s, 1H), 8.30 (t, J=5.5 Hz, 1H), 7.96 (s, 1H), 7.85 (s, 1H), 7.54 (d, J=8.3 Hz, 1H), 7.47 (d, J=8.4 Hz, 1H), 7.35 (d, J=8.4 Hz, 1H), 7.21 (d, J=8.4 Hz, 1H), 7.16 (s, 1H), 6.82-6.64 (m, 5H), 6.41 (d, J=2.3 Hz, 1H), 6.26 (d, J=2.4 Hz, 2H), 5.78 (d, J=7.6 Hz, 1H), 5.63 (s, 1H), 5.25 (d, J=7.5 Hz, 2H), 5.21-5.14 (m, 2H), 5.12 (s, 1H), 4.89 (s, 1H), 4.68 (d, J=6.9 Hz, 1H), 4.50-4.34 (m, 4H), 4.24-4.02 (m, 3H), 3.68 (d, J=10.8 Hz, 1H), 3.32-3.24 (m, 3H), 3.23-3.07 (m, 3H), 2.87 (s, 2H), 2.74 (s, 2H), 2.23-1.85 (m, 5H), 1.62-1.47 (m, 1H), 1.29 (s, 3H), 1.07 (d, J=6.3 Hz, 4H), 0.92 (d, J=5.0 Hz, 3H), 0.86 (d, J=4.8 Hz, 3H);

HRMS (ESI) m/z calcd for C₈₁H₉₂Cl₃N₁₃O₂₇ [M+2H]²⁺ 892.7719, found 892.7714.

### Pharmacology

The MIC of target compounds were tested at JMI Laboratories (North Liberty, Iowa, US) as shown in Table 1. The test compounds including vancomycin hydrochloride were supplied as a 6.4 mg/mL solution in 100% dimethyl sulfoxide (DMSO) aliquoted into individual wells of a 96-well plate (50 μL per well). Each compound was diluted and tested in a 12-point, 2-fold dilution series from 0.03-64 μg/mL. Standardized inocula were added to the MIC test plates, which were incubated for the prescribed times at 35° C. The N. gonorrhoeae strain was incubated in an atmosphere containing 5% CO₂.

The final optical density of each well at 600 nm was measured using a Tecan Spark plate reader. The compound concentration versus OD600 data were then curve fit using custom software. An MIC value was defined as the lowest compound concentration that led to an OD600 value≤0.15 for the E. coli, K. pneumoniae, P. aeruginosa, A. baumannii, or S. aureus strains. For the N. gonorrhoeae strain, an OD600 value≤0.19 was used as a cutoff to define an MIC value; this higher cut-off value was required because the N. gonorrhoeae MIC plates were covered with plastic seals that increased the background OD600 value. These OD600 cut-off values produced MIC values that agreed with corresponding MIC values that were read by eye.

JMI Laboratories followed current CLSI quality assurance practices when performing the susceptibility tests. MIC values were validated by concurrently testing the CLSI-recommended ATCC QC reference strains S. aureus ATCC 29213 and K. pneumoniae ATCC 700603. The inoculum density during susceptibility testing was monitored by bacterial colony counts. Where applicable, QC ranges for tested reference strains were those ranges approved or published by CLSI for testing in CAMHB. The MIC values for erythromycin and tetracycline were identical or nearly identical when tested in the original strain validation and during the testing of this compound set. Importantly, the CLSI reference method for testing N. gonorrhoeae is the agar dilution MIC method, which was not used here. Thus, CLSI MIC QC ranges for N. gonorrhoeae could not be applied to the antimicrobials studied here.

The specific pharmacological responses observed may vary according to and depending on the particular active compound selected or whether there are present pharmaceutical carriers, as well as the type of composition and mode of administration employed, and such expected variations or differences in the results are contemplated in accordance with practice of the present disclosure.

TABLE 1
In vitro activities of vancomycin derivatives
against 8-strain bacterial panel

| | MIC (μg/mL) | | | | | | | |
|---|---|---|---|---|---|---|---|---|
| Com- | Gram-negative | | | | | | | Gram |
| pound | | EC | | | PSA | | | positive |
| Number | EC | Efflux | KPN | PSA | Efflux | ACB | GC | SA |
| 1 | 64 | 64 | >64 | >64 | >64 | 32 | >64 | 8 |
| 2 | 32 | 32 | >64 | >64 | >64 | 32 | >64 | 8 |
| 3 | 16 | >64 | >64 | >64 | >64 | 2 | >64 | 4 |
| 4 | 8 | >64 | >64 | >64 | >64 | 1 | >64 | 2 |
| 5 | 8 | >64 | >64 | >64 | >64 | 1 | >64 | 4 |
| 6 | 64 | >64 | >64 | >64 | >64 | 4 | >64 | 4 |
| 7 | 64 | 64 | >64 | >64 | >64 | 1 | >64 | 4 |
| 8 | 64 | 64 | >64 | >64 | >64 | 2 | >64 | 4 |
| 9 | 64 | 64 | >64 | >64 | >64 | 8 | >64 | 4 |
| 10 | 16 | >64 | >64 | >64 | >64 | 2 | >64 | 4 |
| 11 | 64 | >64 | >64 | >64 | >64 | 1 | >64 | 4 |
| 12 | 64 | 64 | >64 | >64 | >64 | 4 | >64 | 2 |
| 13 | 64 | >64 | >64 | >64 | >64 | 4 | >64 | 2 |
| 14 | >64 | >64 | >64 | >64 | >64 | 16 | >64 | 4 |
| 15 | >64 | >64 | >64 | >64 | >64 | 16 | >64 | 4 |
| 16 | 32 | 64 | >64 | >64 | >64 | 4 | >64 | 4 |
| 17 | 32 | 64 | >64 | >64 | >64 | 2 | >64 | 2 |
| 18 | 64 | >64 | >64 | >64 | >64 | 1 | >64 | 4 |
| 19 | >64 | >64 | >64 | >64 | >64 | 1 | >64 | 8 |
| 20 | 32 | 64 | >64 | 64 | 64 | 32 | >64 | 32 |
| 21 | 32 | 64 | >64 | 64 | >64 | 32 | >64 | 32 |
| 22 | 16 | 32 | >64 | >64 | >64 | 4 | >64 | 64 |
| 23 | 64 | >64 | >64 | >64 | >64 | 8 | >64 | 64 |
| 24 | >64 | >64 | >64 | >64 | >64 | 8 | >64 | 32 |
| 25 | 16 | 64 | >64 | >64 | >64 | 32 | >64 | 4 |
| 26 | 64 | >64 | >64 | >64 | >64 | 1 | >64 | 16 |
| 27 | 16 | 64 | >64 | >64 | >64 | 16 | >64 | 8 |
| 28 | >64 | >64 | >64 | >64 | >64 | 32 | >64 | 4 |
| 29 | 32 | >64 | >64 | >64 | >64 | 16 | >64 | 4 |
| 30 | 64 | >64 | >64 | >64 | >64 | 32 | >64 | 4 |
| 31 | >64 | 32 | >64 | >64 | >64 | 64 | >64 | 8 |
| 32 | >64 | >64 | >64 | >64 | >64 | 32 | >64 | 32 |

Abbreviations: EC, E. coli (wild-type) BW25113; EC efflux⁻, E. coli (tolC) JW5503-1; KPN, K. pneumoniae ATCC 700603; PSA, P. aeruginosa PAO1; PSA efflux⁻, P. aeruginosa (multi-pump-gene mutant) PAM1626; ACB, A. baumannii JMI #1186154; GC, N. gonorrhoeae WHO-Z; SA, S. aureus ATCC 29213. Broth microdilution MIC values were measured in iron-depleted CAMHB for all strains except for GC, which was tested in fastidious broth.

Although specific embodiments of the present disclosure are herein illustrated and described in detail, the disclosure is not limited thereto. The above detailed descriptions are provided as exemplary of the present disclosure and should not be construed as constituting any limitation of the disclosure. Modifications will be obvious to those skilled in the art, and all modifications that do not depart from the spirit of the disclosure are intended to be included with the scope of the appended claims.

1. A compound of Formula (1) or a pharmaceutically acceptable salt thereof: 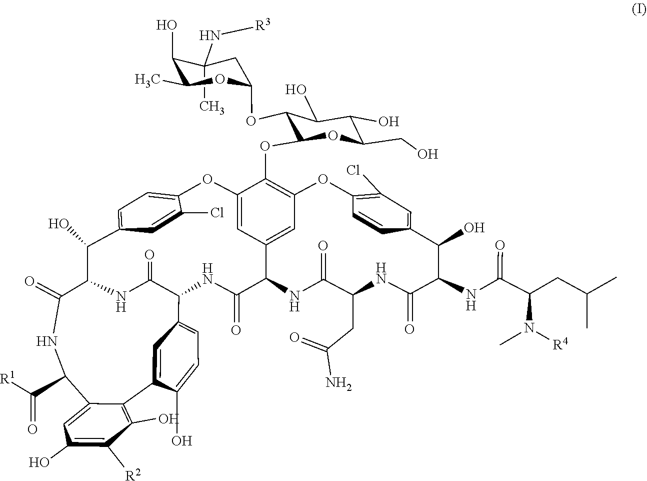
wherein:
(i) R¹ is 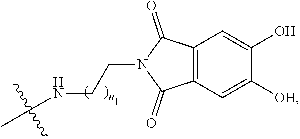
and each of R², R³, and R⁴, is H;
(ii) R¹ is 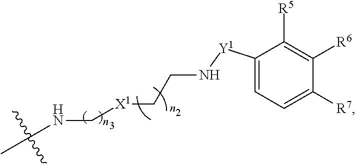
and each of R², R³, and R⁴, is H;
(iii) R¹ is 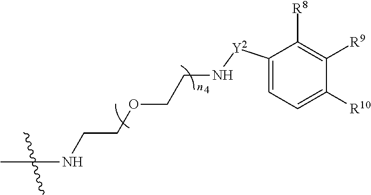
and each of R², R³, and R⁴, is H;
(iv) R¹ is 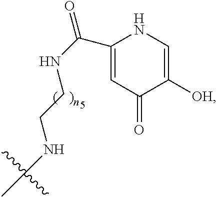
and each of R², R³, and R⁴, is H;
(v) R¹ is OH; R² is 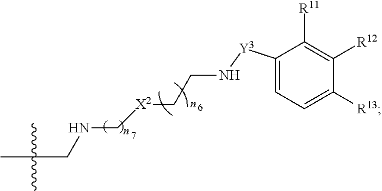
and each of R³ and R⁴ are H;
(vi) R¹ is OH; R³ is 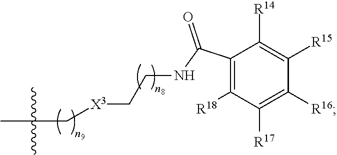
and each of R² and R⁴ are H; or
(vii) R¹ is OH; R⁴ is 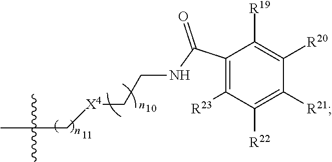
and each of R² and R³ are H; and wherein:
X¹ is a bond or 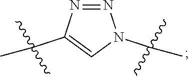 [SMILES: *c1cn(*)nn1]
X² is a bond or 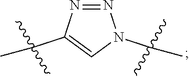 [SMILES: *c1cn(*)nn1]
X³ is a bond or 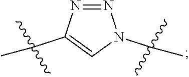 [SMILES: *c1cn(*)nn1]
X⁴ is a bond or 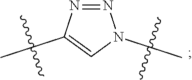 [SMILES: *c1cn(*)nn1]
Y¹ is C(O) or SO₂;
Y² is C(O) or SO₂;
Y³ is C(O) or SO₂;
n₁ is 1-5;
n₂ is 1-10;
n₃ is 0-5;
n₄ is 1-5;
n₅ is 1-5;
n₆ is 1-10;
n₇ is 0-5;
n₈ is 0-5;
n₉ is 0-5;
n₁₀ is 0-5;
n₁₁ is 0-5;
R⁵ is selected from the group consisting of hydrogen, halogen, and OH;
R⁶ is selected from the group consisting of hydrogen, halogen, and OH;
R⁷ is selected from the group consisting of hydrogen, halogen, and OH;
R⁸ is selected from the group consisting of hydrogen, halogen, and OH;
R⁹ is selected from the group consisting of hydrogen, halogen, and OH;
R¹⁰ is selected from the group consisting of hydrogen, halogen, and OH;
R¹¹ is selected from the group consisting of hydrogen, halogen, and OH;
R¹² is selected from the group consisting of hydrogen, halogen, and OH;
R¹³ is selected from the group consisting of hydrogen, halogen, and OH;
R¹⁴ is selected from the group consisting of hydrogen, halogen, and OH;
R¹⁵ is selected from the group consisting of hydrogen, halogen, and OH;
R¹⁶ is selected from the group consisting of hydrogen, halogen, and OH;
R¹⁷ is selected from the group consisting of hydrogen, halogen, and OH;
R¹⁸ is selected from the group consisting of hydrogen, halogen, and OH;
R¹⁹ is selected from the group consisting of hydrogen, halogen, and OH;
R²⁰ is selected from the group consisting of hydrogen, halogen, and OH;
and R²¹ is selected from the group consisting of hydrogen, halogen, and OH.

2. The compound of claim 1, wherein the halogen is Cl.

3. The compound of claim 1, wherein R¹ is 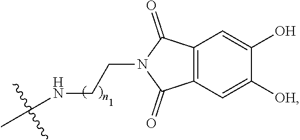
and each of R², R³, and R⁴, is H.

4. The compound of claims 1 and 3, wherein n₁ is 1.

5. The compound of claims 1 and 3, wherein n₁ is 3.

6. The compound of claim 1, wherein R¹ is 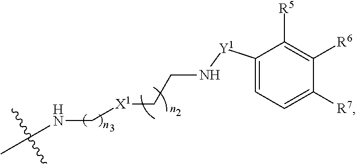
and each of R², R³, and R⁴, is H.

7. The compound of claims 1 and 6, wherein n₂ is 1, n₃ is 0, X¹ is a bond, Y¹ is C(O), R⁵ is Cl, R⁶ is OH, and R⁷ is OH.

8. The compound of claims 1 and 6, wherein n₂ is 2 n₃ is 0, X¹ is a bond, Y¹ is C(O), R⁵ is Cl, R⁶ is OH, and R⁷ is OH.

9. The compound of claims 1 and 6, wherein n₂ is 3, n₃ is 0, X¹ is a bond, Y¹ is C(O), R⁵ is Cl, R⁶ is OH, and R⁷ is OH.

10. The compound of claims 1 and 6, wherein n₂ is 4, n₃ is 0, X¹ is a bond, Y¹ is C(O), R⁵ is Cl, R⁶ is OH, and R⁷ is OH.

11. The compound of claims 1 and 6, wherein n₂ is 5, n₃ is 0, X¹ is a bond, Y¹ is C(O), R⁵ is Cl, R⁶ is OH, and R⁷ is OH.

12. The compound of claims 1 and 6, wherein n₂ is 6, n₃ is 0, X¹ is a bond, Y¹ is C(O), R⁵ is Cl, R⁶ is OH, and R⁷ is OH.

13. The compound of claims 1 and 6, wherein n₂ is 7, n₃ is 0, X¹ is a bond, Y¹ is C(O), R⁵ is Cl, R⁶ is OH, and R⁷ is OH.

14. The compound of claims 1 and 6, wherein n₂ is 2, n₃ is 0, X¹ is a bond, Y¹ is C(O), R⁵ is OH, R⁶ is OH, and R⁷ is H.

15. The compound of claims 1 and 6, wherein n₂ is 3, n₃ is 0, X¹ is a bond, Y¹ is C(O), R⁵ is OH, R⁶ is OH, and R⁷═H.

16. The compound of claims 1 and 6, wherein n₂ is 1, n₃ is 1, X¹ is 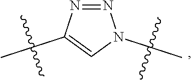 [SMILES: *c1cn(*)nn1]
Y¹ is C(O), R⁵ is OH, R⁶ is OH, and R⁷ is H.

17. The compound of claims 1 and 6, wherein n₂ is 1, n₃ is 2, X¹ is 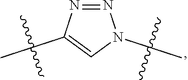 [SMILES: *c1cn(*)nn1]
Y¹ is C(O), R⁵ is OH, R⁶ is OH, and R⁷ is H.

18. The compound of claims 1 and 6, wherein n₂ is 2, n₃ is 0, X¹ is a bond, Y¹ is SO₂, R⁵ is H, R⁶ is OH, and R⁷ is OH.

19. The compound of claims 1 and 6, wherein n₂ is 3, n₃ is 0, X¹ is a bond, Y¹ is SO₂, R⁵ is H, R⁶ is OH, and R⁷ is OH.

20. The compound of claims 1 and 6, wherein n₂ is 2, n₃ is 1, X¹ is 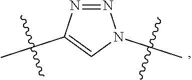 [SMILES: *c1cn(*)nn1]
Y¹ is C(O), R⁵ is Cl, R⁶ is OH, and R⁷ is OH.

21. The compound of claim 1, wherein R¹ is 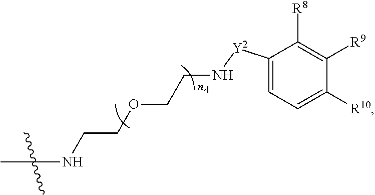
and each of R², R³, and R⁴, is H.

22. The compound of claims 1 and 20, wherein n₄ is 1, Y² is C(O), R⁸ is Cl, R⁹ is OH, and R¹⁰ is OH.

23. The compound of claims 1 and 20, wherein n₄ is 2, Y² is C(O), R⁸ is Cl, R⁹ is OH, and R¹⁰ is OH.

24. The compound of claims 1 and 20, wherein n₄ is 2, Y² is SO₂, R⁸ is H, R⁹ is OH, and R¹⁰ is OH.

25. The compound of claim 1, wherein R¹ is 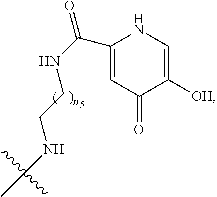
and each of R², R³, and R⁴, is H.

26. The compound of claims 1 and 25, wherein n₅ is 2.

27. The compound of claims 1 and 25, wherein n₅ is 3.

28. The compound of claim 1, wherein R¹ is OH; R² is 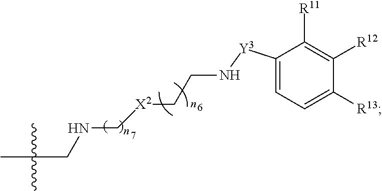
and each of R³ and R⁴ are H.

29. The compound of claims 1 and 28, wherein n₆ is 2, n₇ is 0, X² is a bond, Y³ is C(O), R¹¹ is Cl, R¹² is OH, and R¹³ is OH.

30. The compound of claims 1 and 28, wherein n₇ is 3, n₇ is 0, X² is a bond, Y³ is C(O), R¹¹ is Cl, R¹² is OH, and R¹³ is OH.

31. The compound of claims 1 and 28, wherein n₇ is 1, n₇ is 0, X² is a bond, Y³ is C(O), R¹¹ is OH, R¹² is OH, and R¹³ is H.

32. The compound of claims 1 and 28, wherein n₇ is 1, n₇ is 0, X² is a bond, Y³ is SO₂, R¹¹ is H, R¹² is OH, and R¹³ is OH.

33. The compound of claims 1 and 28, wherein n₇ is 2, n₇ is 1, X² is 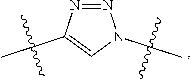 [SMILES: *c1cn(*)nn1]
Y³ is C(O), R¹¹ is Cl, R¹² is OH, and R¹³ is H.

34. The compound of claim 1, wherein R¹ is OH; R³ is 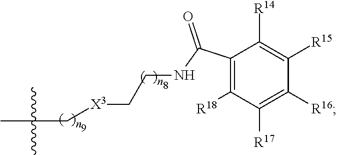
and each of R² and R⁴ are H.

35. The compound of claims 1 and 34, wherein n₉ is 1, n₉ is 0, X³ is a bond, R¹⁴ is Cl, R¹⁵ is OH, R¹⁶ is OH, R¹⁷ is H, and R¹⁸ is H.

36. The compound of claims 1 and 34, wherein n₉ is 2, n₉ is 0, X³ is a bond, R¹⁴ is Cl, R¹⁵ is OH, R¹⁶ is OH, R¹⁷ is H, and R¹⁸ is H.

37. The compound of claims 1 and 34, wherein ng is 2, n₉ is 3, X³=is 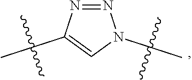 [SMILES: *c1cn(*)nn1]
R¹⁴ is H, R¹⁵ is H, R¹⁶ is OH, R¹⁷ is OH, and R¹⁸ is Cl.

38. The compound of claim 1, wherein R¹ is OH; R⁴ is 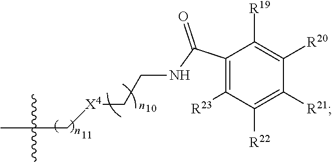
and each of R² and R³ are H.

39. The compound of claims 1 and 39, wherein n₁₀ is 1, n₁₁ is 0, X⁴ is a bond, R¹⁹ is Cl, R²⁰ is OH, R²¹ is OH, R²² is H, and R²³ is H.

40. The compound of claims 1 and 39, wherein n₁₀ is 1, n₁₁ is 3, X⁴ is 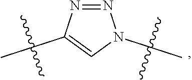 [SMILES: *c1cn(*)nn1]
R¹⁹ is H, R²⁰ is H, R²¹ is OH, R²² is OH, and R²³ is Cl.

41. A compound or a pharmaceutically acceptable salt thereof selected from the group consisting of

| Com- | |
|---|---|
| pound # | Compound |
| 1 | 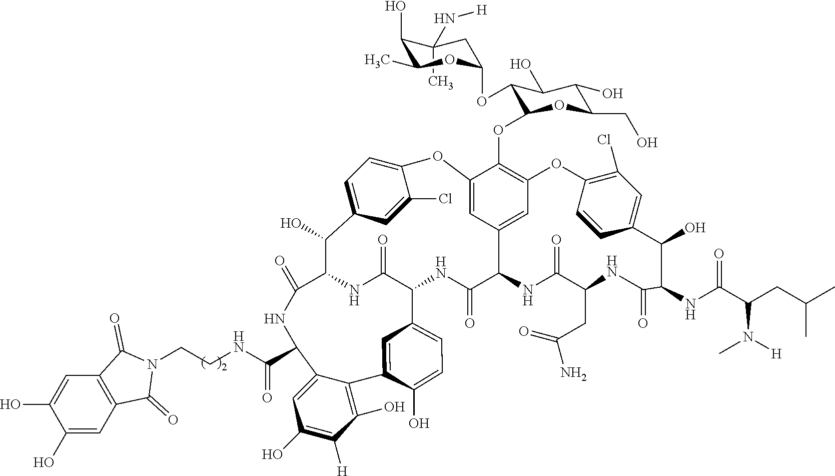 |
| 2 | 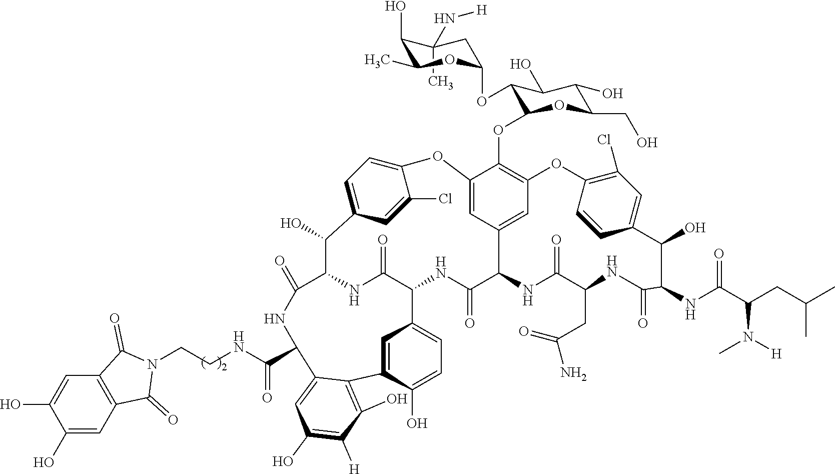 |
| 3 | 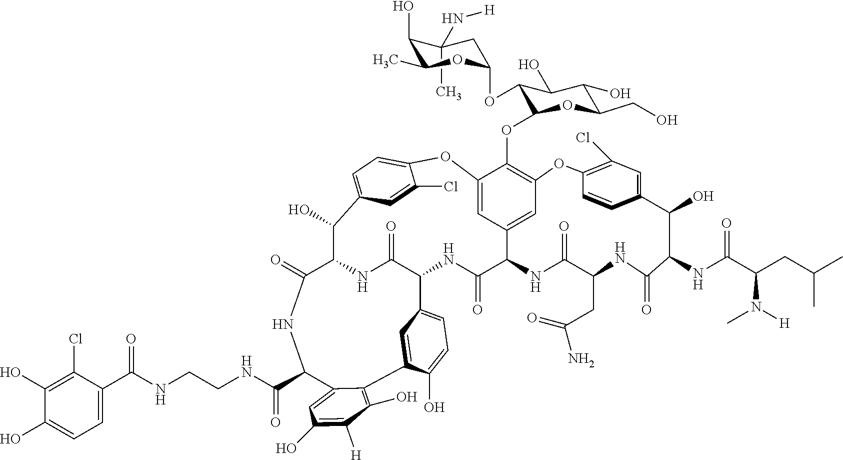 |
| 4 | 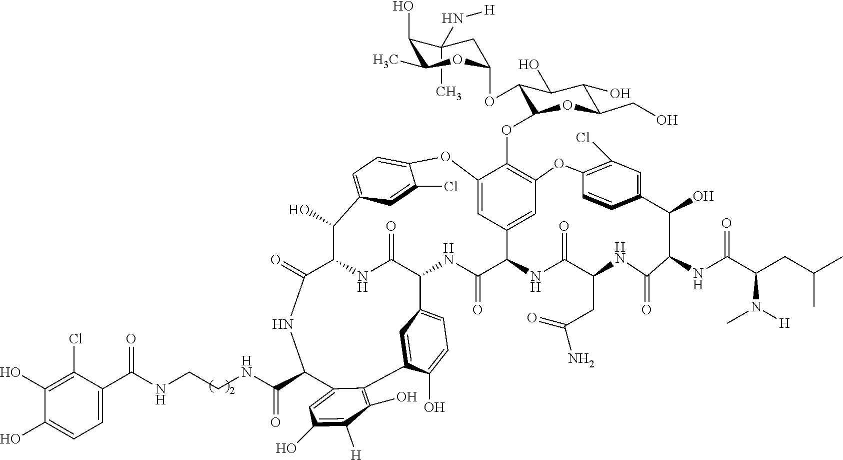 |
| 5 | 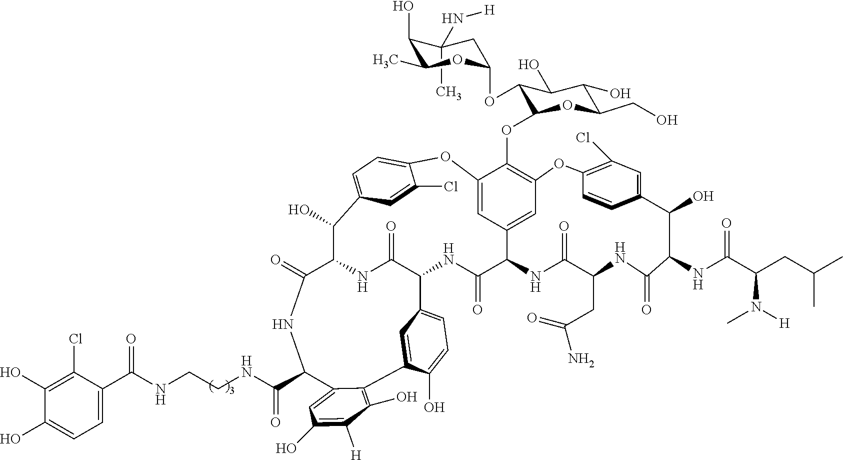 |
| 6 | 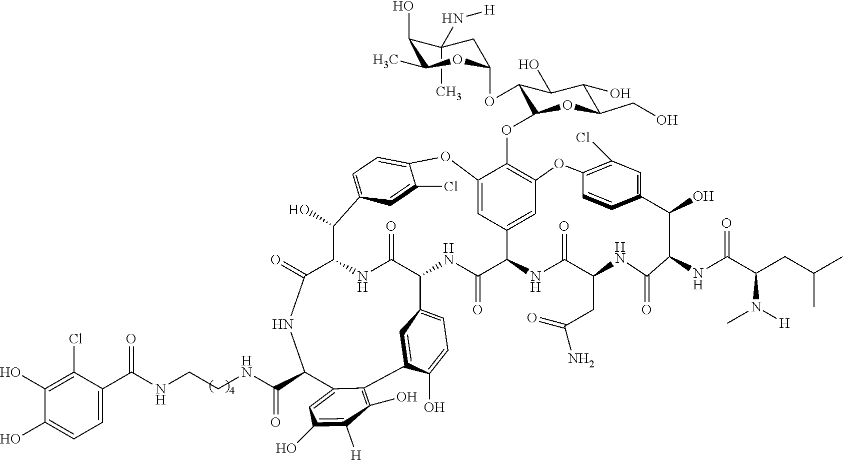 |
| 7 | 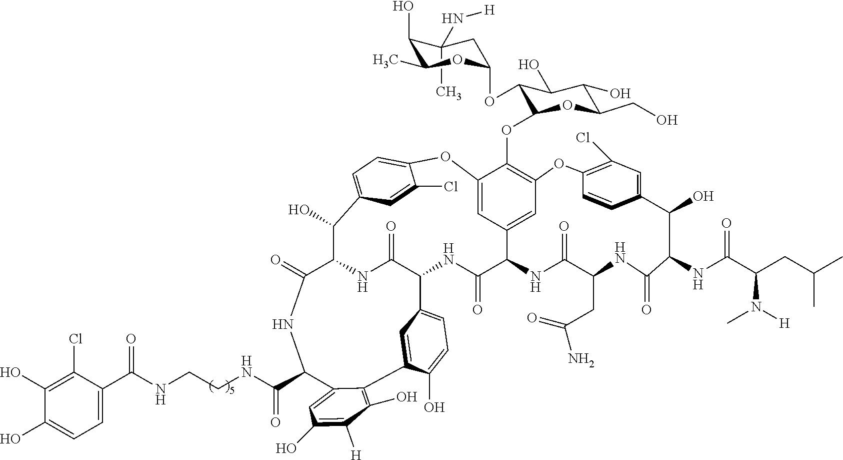 |
| 8 | 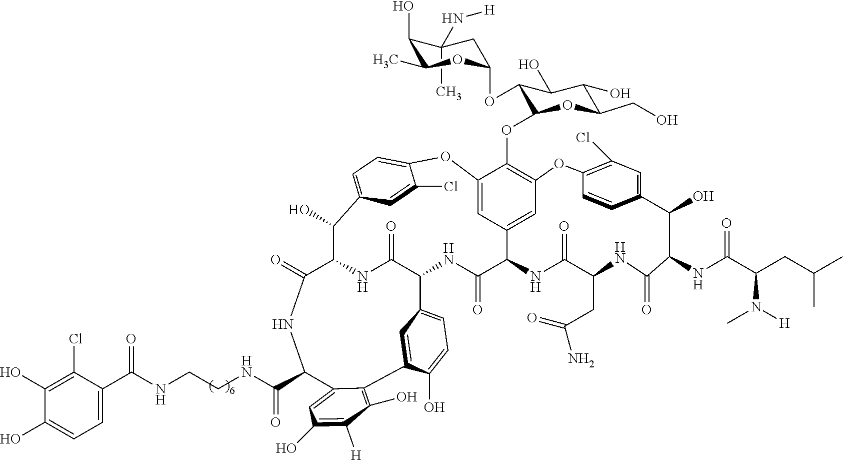 |
| 9 | 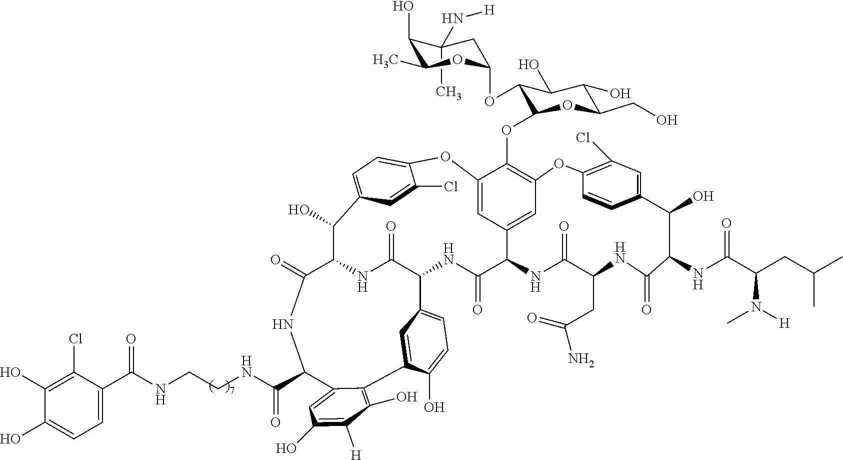 |
| 10 | 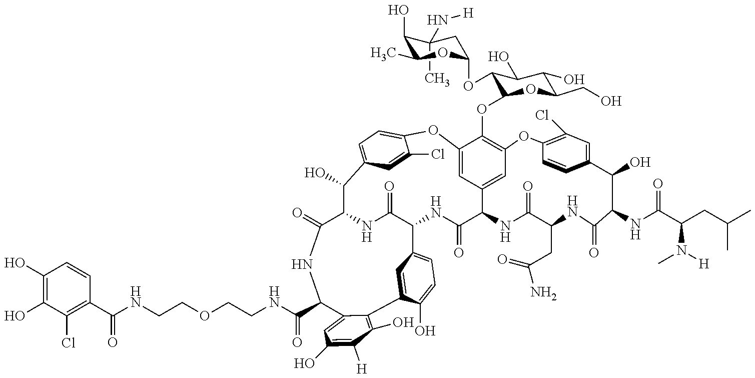 |
| 11 | 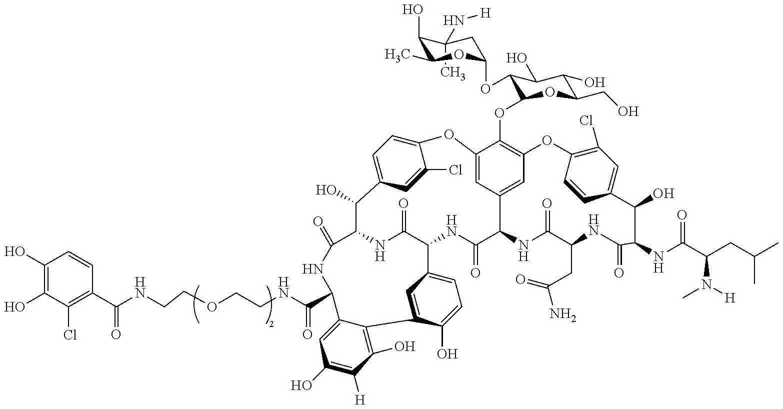 |
| 12 | 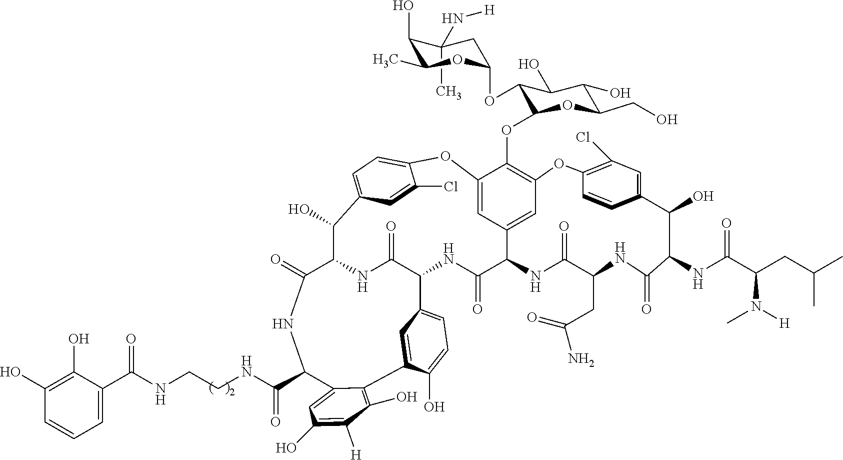 |
| 13 | 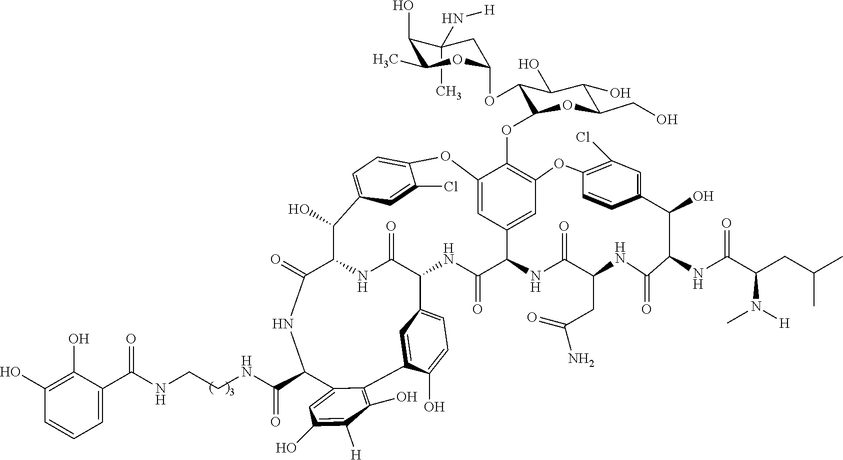 |
| 14 | 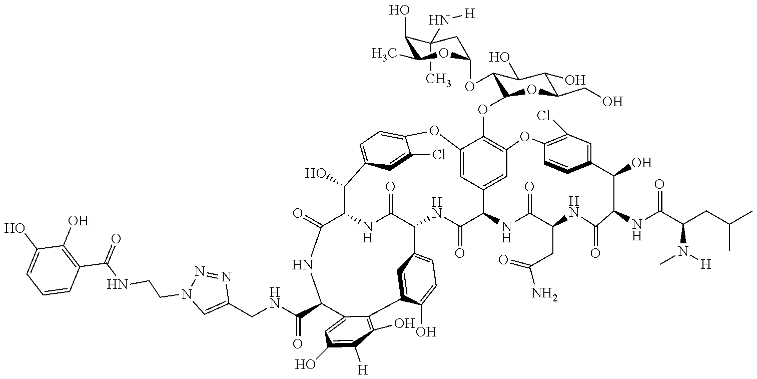 |
| 15 | 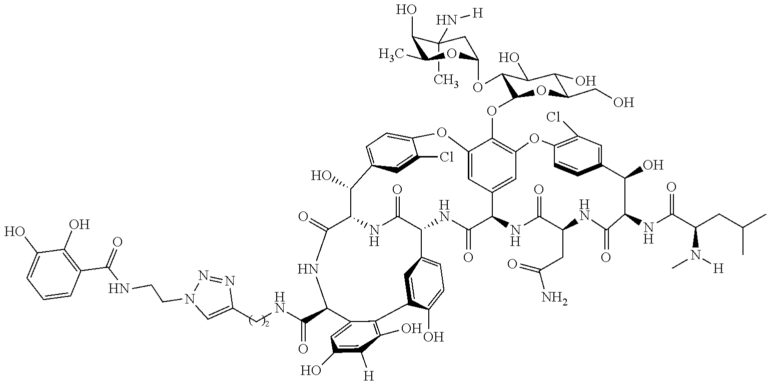 |
| 16 | 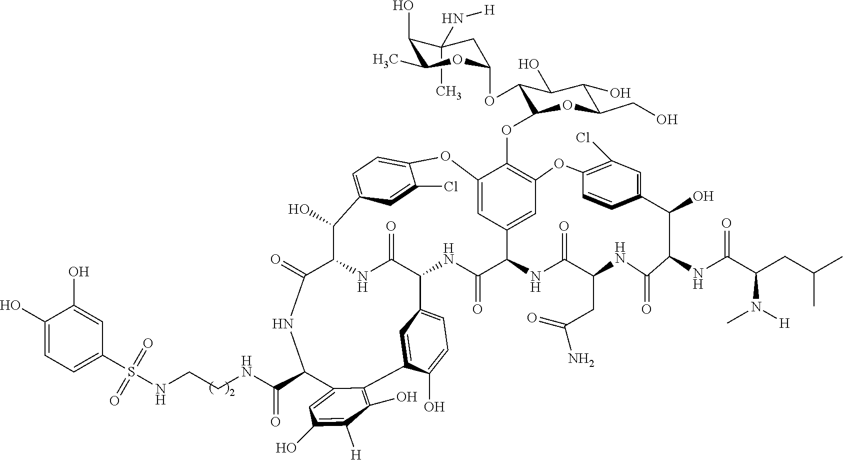 |
| 17 | 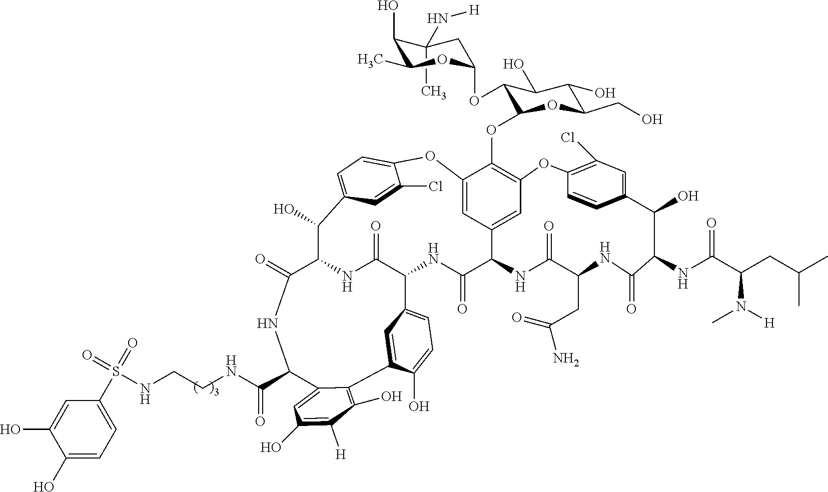 |
| 18 | 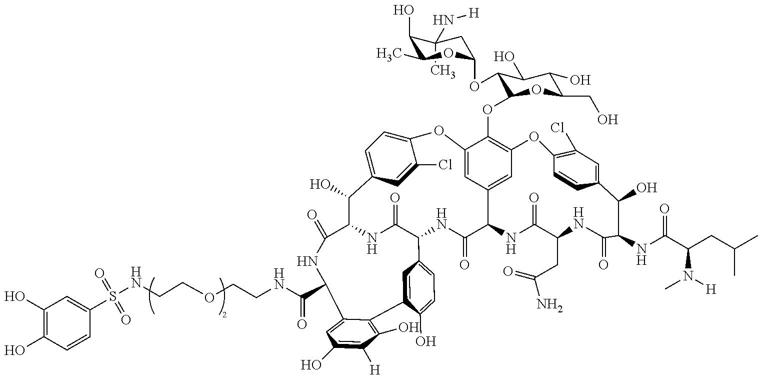 |
| 19 | 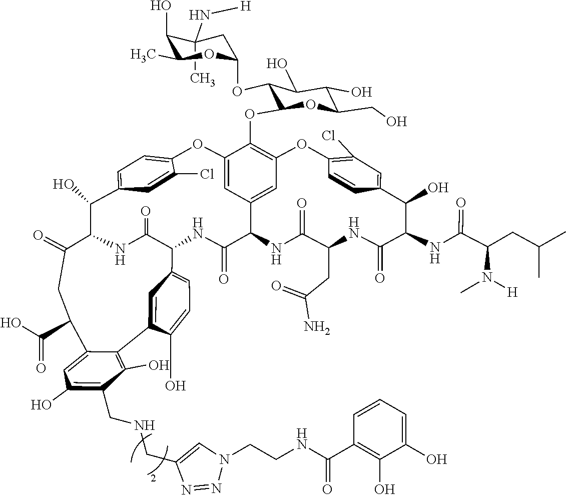 |
| 20 | 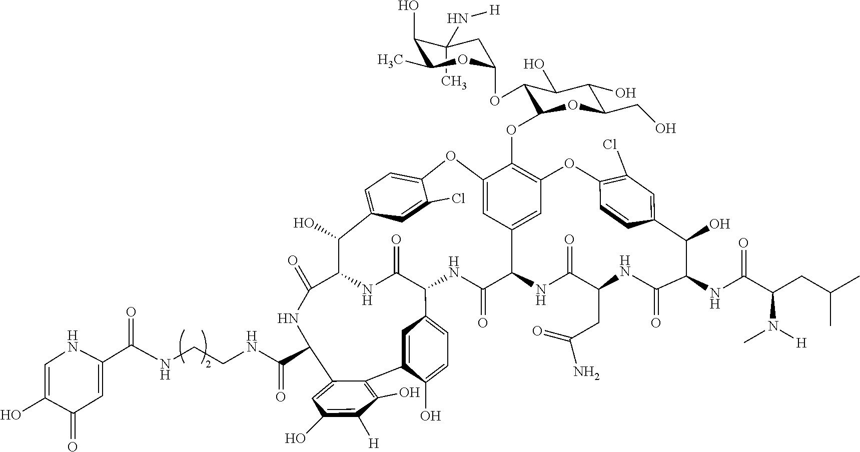 |
| 21 | 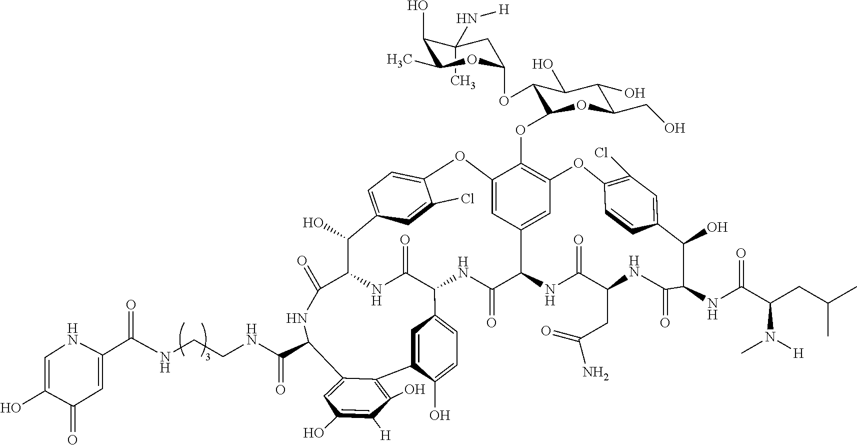 |
| 22 | 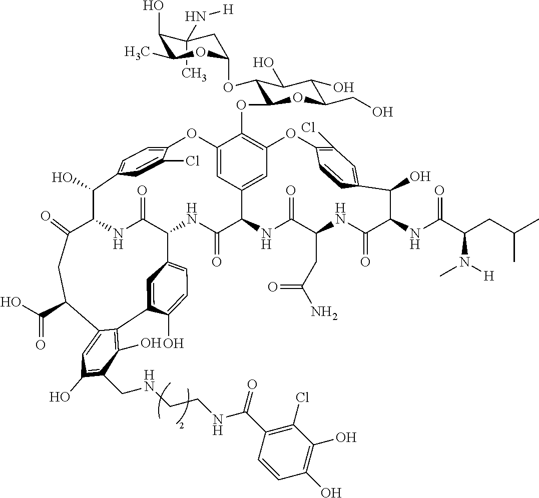 |
| 23 | 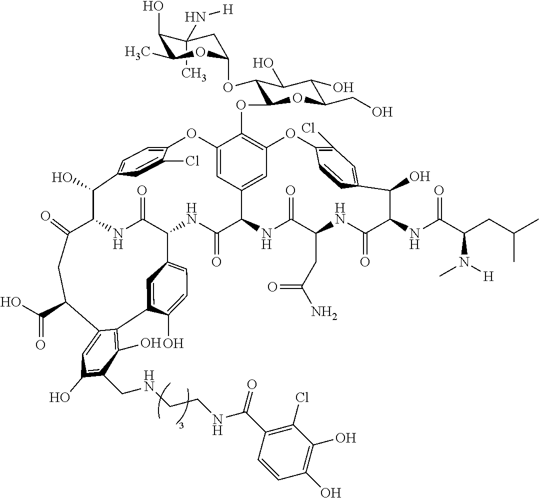 |
| 24 | 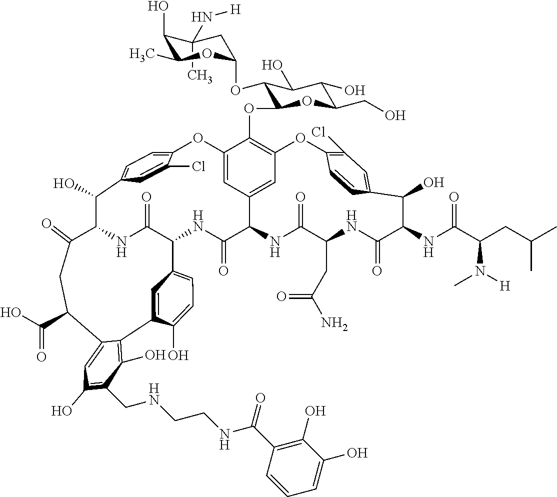 |
| 25 | 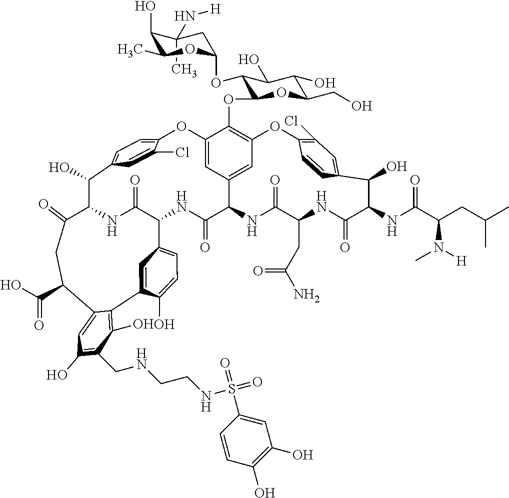 |
| 26 | 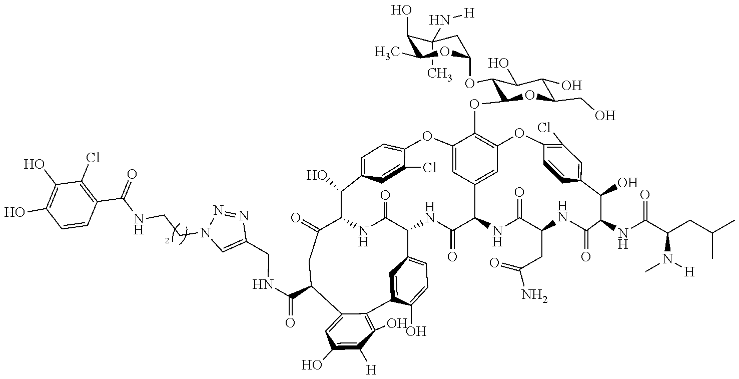 |
| 27 | 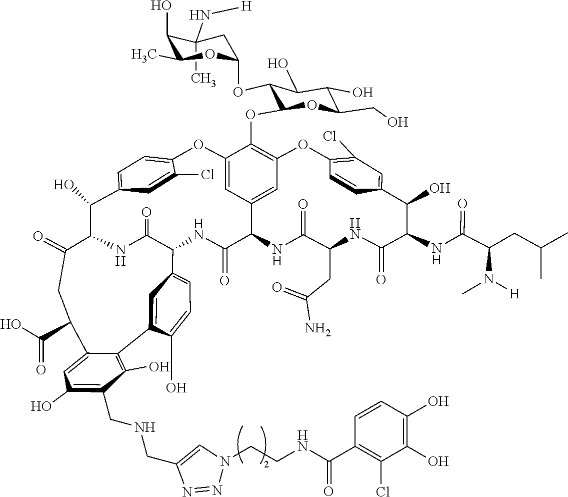 |
| 28 | 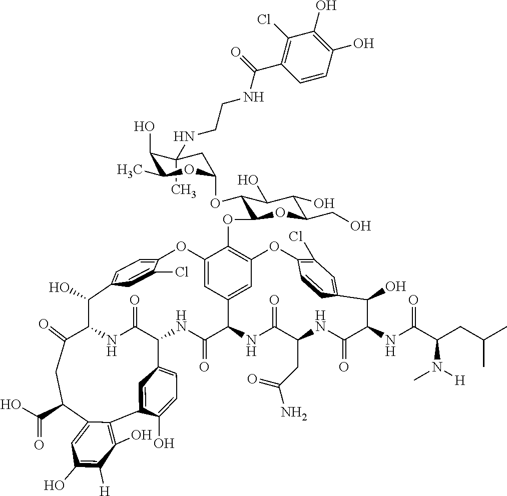 |
| 29 | 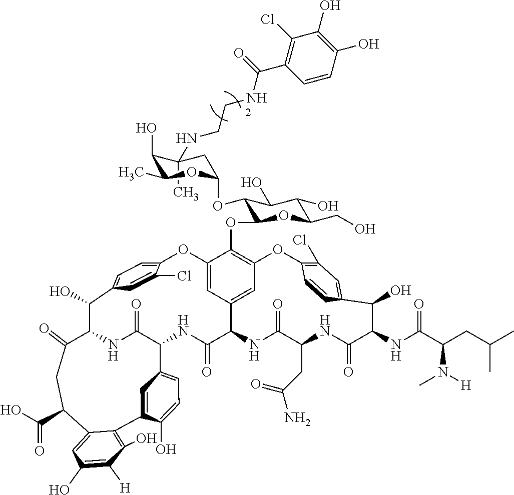 |
| 30 | 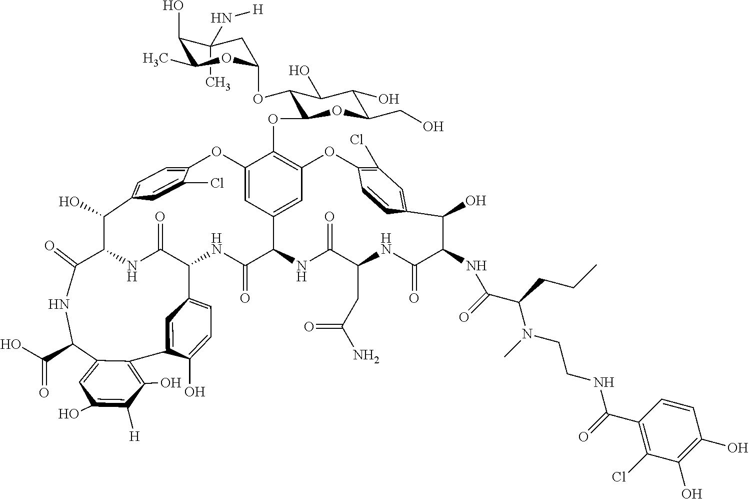 |
| 31 | 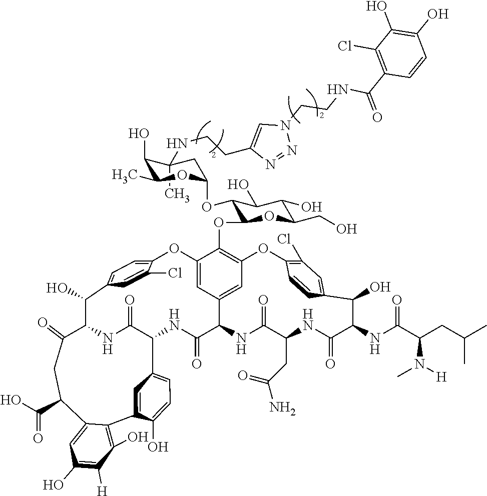 |
| 32 | 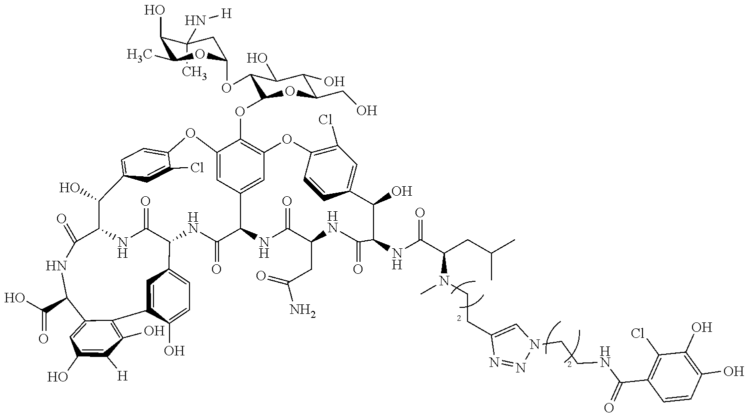 |

or a pharmaceutically acceptable salt thereof.

42. A pharmaceutical composition comprising any of the compounds of claims 1-41 or a pharmaceutically acceptable salt thereof.

43. A pharmaceutical composition comprising any of the compounds of claims 1-41 and a pharmaceutically acceptable carrier.

44. A pharmaceutical composition comprising a therapeutically effective amount of the compounds of claims 1-41 or a pharmaceutically acceptable salt thereof and one or more pharmaceutically acceptable carriers or vehicles.

45. The compounds of claims 1-41 or the pharmaceutical composition of claims 42-44 for use in the treatment of a bacterial infectious disease.

46. A method of treating a bacterial infection in a patient in need thereof, comprising administering to the patient the compounds of claims 1-41 or the composition of claims 42-44.

47. A method of treating a bacterial infection in a patient in need thereof, comprising administering to the patient the compounds of claims 1-41 or the composition of claims 42-44 in combination with another treatment agent.

48. A method of controlling, treating, or reducing the advancement, severity, or effects of a bacterial infection in a patient, comprising administering to said patient a therapeutically effective around of the compounds claims 1-41 or the pharmaceutical compostions of claims 42-44.

49. A method of decreasing or inhibiting bacterial quantity in a biological sample, comprising contacting said biological sample with a compound of claims 1-41 or a composition of claims 42-44.

50. The method of claim 47, wherein the bacteria comprises a Gram-positive bacteria or Gram-negative bacteria.

51. The method of claim 47, wherein the bacteria comprises a Gram-positive bacteria.

52. The method of claim 47, wherein the bacteria comprises a Gram-negative bacteria.

53. The method of claim 52, wherein the Gram-negative bacteria comprises Acinetobacter baumannii and Escherichia coli.

54. Use of a compound of any one of claims 1-41 or the pharmaceutical composition of claims 42-44, in the manufacture of a medicament for the treatment of a bacterial infectious disease in a patient in needed thereof.

55. Use of a compound of any one of claims 1-41 or the pharmaceutical composition of claims 42-44, for the treatment of a bacterial infectious disease associated with Gram-positive bacteria.

56. Use of a compound of any one of claims 1-41 or the pharmaceutical composition of claims 42-44, for the treatment of a bacterial infectious disease associated with Gram-negative bacteria.

57. Use of a compound of any one of claims 1-41 or the pharmaceutical composition of claims 42-44, for the treatment of a bacterial infectious disease associated with Gram-negative bacteria including Acinetobacter baumannii and Escherichia coli.
